(19) United States
(12) Patent Application Publication
(10) Pub. No.: US 2022/0017490 A1
(43) Pub. Date: Jan. 20, 2022

(54) CO-CRYSTALS OF 2-METHYL-1 -[(4-[6-(TRIFLUOROMETHYL)PYRIDIN-2-YL]-6-{[2-(TRIFLUOROMETHYL) PYRIDIN-4-YL]AMINO}-1,3,5-TRIAZIN-2-YL)AMINO]PROPAN-2-OL, COMPOSITIONS AND METHODS OF USE THEREOF

(71) Applicant: Celgene Corporation, Summit, NJ (US)

(72) Inventors: Eric A. Fromme, Flemington, NJ (US); Kevin J. Klopfer, Flemington, NJ (US)

(21) Appl. No.: 17/290,228

(22) PCT Filed: Nov. 1, 2019
(86) PCT No.: PCT/US2019/059398

§ 371 Date: Apr. 30, 2021

Related U.S. Application Data
(60) Provisional application No. 62/755,177, filed on Nov. 2, 2018.

(51) Int. Cl. C07D 401/14; A61K 45/06

(52) U.S. Cl. CPC C07D 401/14; A61K 47/18; C07B 2200/13; A61K 45/06

(57) ABSTRACT

Provided herein are co-crystals comprising Compound 1 and a coformer. Pharmaceutical compositions comprising the co-crystals and methods for treating, preventing and managing disease are also disclosed.

### RELATED APPLICATIONS

This application claims the benefit of U.S. Provisional Application No. 62/755,177, filed Nov. 2, 2018, the disclosure of which is incorporated herein by reference in its entirety.

### FIELD

Provided herein are co-crystals comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol, methods for preparing the cocrystals and formulations comprising the same. In certain embodiments, the co-crystals provided herein are used for treating a proliferative disease, such as cancer, characterized by the presence of a mutant allele of IDH2.

### BACKGROUND ART

A variety of possible solid forms creates potential diversity in physical and chemical properties for a given pharmaceutical compound. The discovery and selection of solid forms are of great importance in the development of an effective, stable and marketable pharmaceutical product.

Co-crystals are crystalline molecular complexes of two or more non-volatile compounds bound together in a crystal lattice by non-ionic interactions. Pharmaceutical co-crystals are co-crystals of a therapeutic compound, e.g., an API, and one or more non-volatile compound(s) (referred to herein as coformer). A coformer in a pharmaceutical co-crystal is typically a pharmaceutically acceptable molecule, such as, for example, food additives, preservatives, pharmaceutical excipients, or other APIs. In recent years, pharmaceutical co-crystals have emerged as a possible alternative approach to enhance physicochemical properties of drug products.

It has been reported that 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol is effective in treating proliferative diseases, including cancers. See, e.g., U.S. Pat. Nos. 9,732,062; 9,738,625; 9,694,013; US Publication Nos. 2017/0157132; 2017/0246174; and International Publication Nos. WO 2015/017821; WO 2016/126798; and WO 2017/066599.

There is a need to develop formulations comprising co-crystals comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol that have good manufacturability, dissolution, stability and bioavailability.

### SUMMARY

In certain embodiments, provided herein are co-crystals comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol or a solvate, hydrate, stereoisomer, prodrug, or clathrate thereof (Compound 1) and a coformer. In one embodiment, the coformer is fumaric acid, succinic acid, benzoic acid, citric acid, nicotinamide, lactamide, 4-hydroxybenzamide, uracil or saccharin.

Also provided herein are methods of preparing the co-crystals provided herein.

Also provided herein are pharmaceutical compositions comprising one or more co-crystals provided herein.

Also provided herein are methods of treating and managing various diseases or disorders comprising administering to a patient a therapeutically effective amount of a co-crystal provided herein.

In certain embodiments, provided herein are methods of treating hematological malignancies or solid tumors, each characterized by the presence of a mutant allele of IDH2 comprising administering a co-crystal provided herein.

In one embodiment, the hematological malignancy is selected from acute myelogenous leukemia (AML), myelodysplastic syndrome (MDS), chronic myelomonocytic leukemia (CMML), myeloid sarcoma, multiple myeloma, lymphoma (e.g., T-cell lymphoma or B-cell lymphoma), angioimmunoblastic T-cell lymphoma (AITL), blastic plasmacytoid dendritic cell neoplasm and myeloproliferative neoplasm (MPN), each characterized by the presence of a mutant allele of IDH2.

In one embodiment, the solid tumor is selected from glioma, melanoma, chondrosarcoma, and cholangiocarcinoma, each characterized by the presence of a mutant allele of IDH2.

In certain embodiments, the co-crystal provided herein is used for oral administration in patients for treating a proliferative disease, such as cancer, characterized by the presence of a mutant allele of IDH2.

In certain embodiments, the co-crystal provided herein is used for oral administration in pediatric patients for treating a proliferative disease, such as cancer, characterized by the presence of a mutant allele of IDH2.

### BRIEF DESCRIPTION OF DRAWINGS

FIG. 1 provides an X-ray Powder Diffraction (“XRPD”) pattern of a mixture of solid Form A and Form 17 of 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol (Compound 1A).

FIG. 2 provides a differential scanning calorimetry profile (DSC)spectrum of a mixture of solid Form A and Form 17 of Compound 1A.

FIG. 3 provides a thermo gravimetric mass spectrum (TGMS) of a mixture of solid Form A and Form 17 of Compound 1A.

FIG. 4 provides a high-performance liquid chromatography (HPLC) profile of a mixture of solid Form A and Form 17 of Compound 1A.

FIG. 5 provides a mass spectrum of a mixture of solid Form A and Form 17 of Compound 1A.

FIG. 6 provides ¹H NMR spectrum of a mixture of solid Form A and Form 17 of Compound 1A.

FIG. 7 provides an overlay of HT-XRPD patterns (from bottom to top): Compound 1A, fumaric acid and Form Fum1 as obtained from the solvent mediated method in ethyl acetate.

FIG. 8 provides a graphical representation of the Whole Powder Pattern Decomposition of Form Fum1.

FIG. 9 provides a DSC spectrum of Form Fum1.

FIG. 10 provides a TGMS spectrum of Form Fum1.

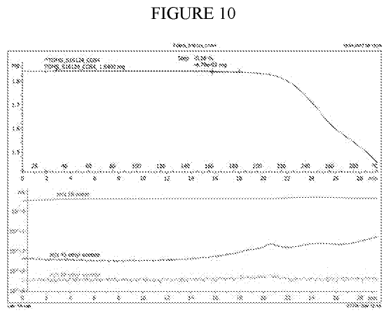

FIG. 11 provides a ¹H-NMR spectrum of Form Fum1.

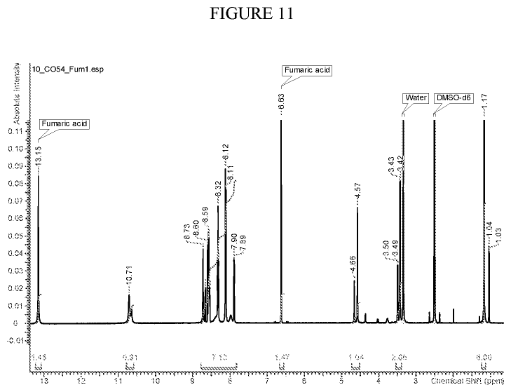

FIG. 12 provides an overlay of HT-XRPD patterns (from bottom to top): Compound 1A, succinic acid, Form Suc1, and Form Suc2+Suc0.

FIG. 13 provides a graphical representation of the Whole Powder Pattern Decomposition of Form Suc1.

FIG. 14 provides a DSC spectrum of Form Suc1.

FIG. 15 provides a TGMS spectrum of Form Suc1.

FIG. 16 provides a ¹H-NMR spectrum of Form Suc1.

FIG. 17 provides an overlay of HT-XRPD patterns (from bottom to top): Compound 1A, nicotinamide, Form Nic1, Forms Nic2+Nic0 and Form Nic3.

FIG. 18 provides a graphical representation of the Whole Powder Pattern Decomposition of Form Nic1.

FIG. 19 provides a DSC spectrum of Form Nic1.

FIG. 20 provides a TGMS spectrum of Form Nic1.

FIG. 21 provides a ¹H-NMR spectrum of Form Nic1.

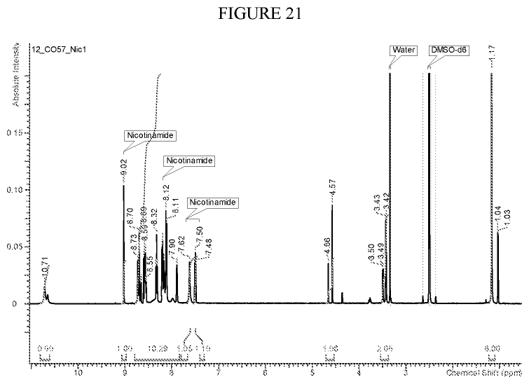

FIG. 22A provides a TGA/SDTA thermograms, and FIG. 22B provides a TGA/MS of the a TGMS spectrum of Form Nic3.

FIG. 23 provides an overlay of HT-XRPD patterns (from bottom to top): Compound 1A, benzoic acid and Ben1 as obtained from sonication in acetonitrile.

FIG. 24 provides a graphical representation of the Whole Powder Pattern Decomposition of Form Ben1.

FIG. 25 provides a DSC spectrum of form Ben1.

FIG. 26 provides a TGMS spectrum of Form Ben1.

FIG. 27 provides a ¹H-NMR spectrum of Form Ben1.

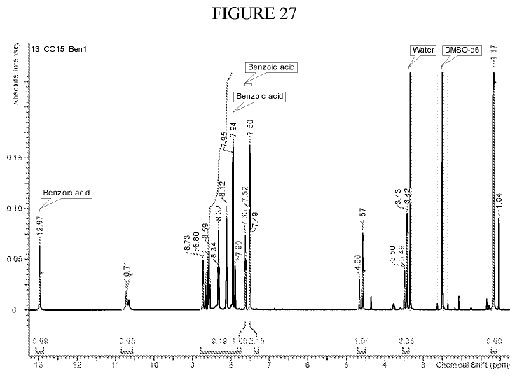

FIG. 28 provides an overlay of HT-XRPD patterns (from bottom to top): Compound 1A, uracil and Form Ura1.

FIG. 29 provide a high resolution XRPD of Form Ura1.

FIG. 30 provides a DSC spectrum of Form Ura1.

FIG. 31 provides a TGMS spectrum of Form Ura1.

FIG. 32 provides a ¹H-NMR spectrum of Ura1.

FIG. 33 provides an overlay of HT-XRPD patterns (from bottom to top): Compound 1A, saccharin and Form Sac1.

FIG. 34 provides a graphical representation of the Whole Powder Pattern Decomposition of Form Sac1.

FIG. 35 provides a DSC spectrum of Form Sac1.

FIG. 36 provides a TGMS spectrum of Form Sac1.

FIG. 37 provides a ¹H-NMR spectrum of Form Sac1.

FIG. 38 provides an overlay of HT-XRPD patterns (from bottom to top): Compound 1A, citric acid, Form Cit1, Form Cit2, Form Cit3 and Form Cit4.

FIG. 39 provides a graphical representation of the Whole Powder Pattern Decomposition of Form Cit3.

FIG. 40 provides a DSC spectrum of Form Cit3.

FIG. 41 provides a TGMS spectrum of Form Cit3.

FIG. 42 provides a ¹H-NMR spectrum of Form Cit3.

FIG. 43 provides a graphical representation of the Whole Powder Pattern Decomposition of Form Cit4.

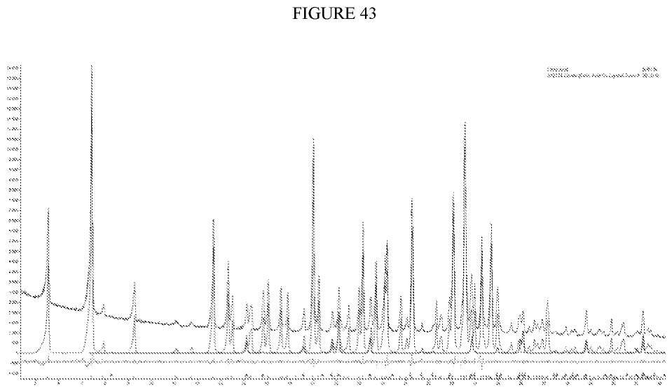

FIG. 44 provides a DSC spectrum of Form Cit4.

FIG. 45 provides a TGMS spectrum of Form Cit4.

FIG. 46 provides a ¹H-NMR spectrum of Form Cit4.

FIG. 47 provides an overlay of HT-XRPD patterns (from bottom to top): Compound 1A, lactamide, Form Lac1, Form Lac2 and Forms Lac3+Lac0.

FIG. 48 provides a DSC spectrum of Form Lac1.

FIG. 49 provides an overlay of HT-XRPD patterns (from bottom to top): Compound 1A, 4-hydroxybenzamide, Form Hbe1, Form Hbe2 and Forms Hbe3+Hbe0.

FIG. 50 provides a graphical representation of the Whole Powder Pattern Decomposition of Form Hbe1.

FIG. 51 provides a DSC spectrum of Form Hbe1.

FIG. 52 provides a TGMS spectrum of Form Hbe1.

FIG. 53 provides a ¹H-NMR spectrum of Form Hbe1.

FIG. 54A provides TGA/SDTA thermograms and FIG. 54B provides a TGA/MS spectrum of Form Hbe2.

FIG. 55 provides an XRPD pattern for solid Form A of Compound 1A.

FIG. 56 provides TGA/DSC spectra for solid Form A of Compound 1A.

FIG. 57 provides ¹H NMR spectrum for solid Form A of Compound 1A.

FIG. 58 provides an XRPD pattern for solid Form G of Compound 1A.

FIG. 59 provides TGA/DSC spectra for solid Form G of Compound 1A.

FIG. 60 provides ¹H NMR spectrum for solid Form G of Compound 1A.

FIG. 61 provides an XRPD pattern for solid Form K of Compound 1A.

FIG. 62 provides TGA/DSC spectra for solid Form K of Compound 1A.

FIG. 63 provides an XRPD pattern for solid Form FB3 of Compound 1A.

FIG. 64 provides an XRPD pattern for solid Form FB7 of Compound 1A.

### DETAILED DESCRIPTION

The details of construction and the arrangement of components set forth in the following description or illustrated in the drawings are intended to describe non-limiting embodiments. Other embodiments and different ways to practice the invention are expressly included. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### Definitions

As used above, and throughout the description of the invention, the following terms, unless otherwise indicated, shall be understood to have the following meanings.

As used in this application, the singular form “a”, “an” and “the” include plural references unless the context clearly dictates otherwise. For example, the term “an intragranular excipient” includes one or more intragranular excipients.

“Compound 1” is meant to describe 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol or solvates, hydrates, stereoisomers, prodrugs, or clathrates thereof 2-Methyl-1-[(4-[6-(trifluoromethyl)pyridin-2-yl]-6-{[2-(trifluoromethyl)pyridin-4-yl]amino}-1,3,5-triazin-2-yl)amino]propan-2-ol methanesulfonate (or mesylate) is also known as enasidenib.

The terms “AG 221” or “AG221” refer to 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol, including solid forms thereof, or 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol methanesulfonate, including solid forms thereof.

2-Methyl-1-[(4-[6-(trifluoromethyl)pyridin-2-yl]-6-{[2-(trifluoromethyl)pyridin-4-yl]amino}-1,3,5-triazin-2-yl)amino]propan-2-ol is currently marketed in the U.S. by Celgene Corporation, as once-daily oral tablets for the treatment of adult patients with relapsed or refractory acute myeloid leukemia (AML) who have an IDH2 mutation, under the trade name IDHIFA®.

“Compound 1A” as used herein refers 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol, including solid forms thereof.

The term “solid form” refers a crystal form or an amorphous form or a mixture thereof of 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol. Certain solid forms of 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol are described in U.S. Pat. No. 9,738,625 and International Publication No. WO 2016/126798, each of which is incorporated by reference in its entirety.

Unless otherwise specified, the term “crystalline” and related terms used herein, when used to describe a substance, component, product, or form, mean that the substance, component, product, or form is substantially crystalline, for example, as determined by X-ray diffraction. (see, e.g., Remington's Pharmaceutical Sciences, 18^{th} ed., Mack Publishing, Easton Pa., 173 (1990); The United States Pharmacopeia, 23^{rd} ed., 1843-1844 (1995)).

Unless otherwise specified, the term “crystal form,” “crystal forms,” and related terms herein refer to crystalline modifications comprising a given substance, including single-component crystal forms and multiple-component crystal forms, and including, but not limited to, polymorphs, solvates, hydrates, other molecular complexes, salts, solvates of salts, hydrates of salts, and polymorphs thereof. In some embodiments, a crystal form of a substance may be substantially free of amorphous forms and/or other crystal forms. In other embodiments, a crystal form of a substance may contain less than about 1%, 2%, 3%, 4%, 5%, 10%, 15%, 20%, 25%, 30%, 35%, 40%, 45%, or 50% of one or more amorphous form(s) and/or other crystal form(s) on a weight basis. Crystal forms of a substance may be obtained by a number of methods. Such methods include, but are not limited to, melt recrystallization, melt cooling, solvent recrystallization, recrystallization in confined spaces such as, e.g., in nanopores or capillaries, recrystallization on surfaces or templates such as, e.g., on polymers, recrystallization in the presence of additives, desolvation, dehydration, rapid evaporation, rapid cooling, slow cooling, vapor diffusion, sublimation, grinding, and solvent-drop grinding.

Unless otherwise specified, the terms “polymorph,” “polymorphic form,” “polymorphs,” “polymorphic forms,” and related terms herein refer to a crystal or a mixture of crystal forms that consist essentially of the same molecule, molecules or ions. Different polymorphs may have different physical properties, such as, for example, melting temperatures, heats of fusion, solubilities, dissolution rates, and/or vibrational spectra as a result of a different arrangement or conformation of the molecules or ions in the crystal lattice. The differences in physical properties exhibited by polymorphs may affect pharmaceutical parameters, such as storage stability, compressibility and density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically a more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing; for example, one polymorph might be more likely to form solvates or might be difficult to filter and wash free of impurities (e.g., particle shape and size distribution might be different between polymorphs).

Unless otherwise specified, the term “cocrystal” or “co-crystal,” as used herein, refers to a crystalline material comprised of two or more non-volatile compounds bond together in a crystal lattice by non-covalent interactions.

Unless otherwise specified, the term “pharmaceutical co-crystal” or “pharmaceutical cocrystal” of an active pharmaceutical ingredient (API), as used herein, refers to a crystalline material comprised of an API and one or more non-volatile compound(s) (referred herein as a coformer). The API and the coformer interact through non-covalent forces in a crystal lattice. In one embodiment, the API is Compound 1. In one embodiment, the API is Compound 1A.

Unless otherwise specified, the terms “solvate” and “solvated,” as used herein, refer to a solid form of a substance which contains solvent. The terms “hydrate” and “hydrated” refer to a solvate wherein the solvent is water. “Polymorphs of solvates” refer to the existence of more than one solid form for a particular solvate composition. Similarly, “polymorphs of hydrates” refer to the existence of more than one solid form for a particular hydrate composition.

Unless otherwise specified, the term “composition” as used herein is intended to encompass a product comprising the specified ingredient(s) (and in the specified amount(s), if indicated), as well as any product which results, directly or indirectly, from combination of the specified ingredient(s) in the specified amount(s).

A “pharmaceutically acceptable excipient, diluent or carrier,” refers to a substance that aids the administration of an active agent to a subject by, for example, modifying the stability of an active agent or modifying the absorption by a subject upon administration. A pharmaceutically acceptable excipient typically has no significant adverse toxicological effect on the patient. Examples of pharmaceutically acceptable excipients include, for example bulking agents, buffers, binders, fillers, disintegrants, lubricants, coatings, sweeteners, flavors, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6^{th} Ed., The Pharmaceutical Press, RPS Publishing (2009). The terms “bulking agent”, and “buffer” are used in accordance with the plain and ordinary meaning within the art.

The term “treat” means decrease, suppress, attenuate, diminish, arrest, or stabilize the development or progression of a disease/disorder (i.e., a cancer such as AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma), lessen the severity of the disease/disorder (i.e., a cancer selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma), each characterized by the presence of a mutant allele of IDH2, or improve the symptoms associated with the disease/disorder (i.e., AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma), each characterized by the presence of a mutant allele of IDH2.

As used herein, and unless otherwise indicated, the terms “manage,” “managing” and “management” encompass preventing the recurrence of the specified disease or disorder in a patient who has already suffered from the disease or disorder, or lengthening the time that a patient who has suffered from the disease or disorder remains in remission. The terms encompass modulating the threshold, development or duration of the disease or disorder, or changing the way that a patient responds to the disease or disorder.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, that provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, “administer” or “administration” refers to the act of physically delivering a substance as it exists outside the body into a subject. Administration includes all forms known in the art for delivering therapeutic agents, including but not limited to oral, topical, mucosal, injections, intradermal, intravenous, intramuscular delivery or other method of physical delivery described herein or known in the art (e.g., implantation of a slow-release device, such as a mini-osmotic pump to a subject; liposomal formulations; buccal; sublingual; palatal; gingival; nasal; vaginal; rectal; intra-arteriole; intraperitoneal; intraventricular; intracranial; or transdermal).

The term “co-administer” as used herein with respect to an additional cancer therapeutic agents means that the additional cancer therapeutic agent may be administered prior to, consecutively with, or following the administration of a composition provided herein. In such combination therapy treatment, the second therapeutic agent(s) is administered by conventional methods.

The terms “subject” and “patient,” are herein used interchangeably and refer to a living organism suffering from one or more of the diseases described herein (e.g., AML) that can be treated by administration of a composition described herein. Non-limiting examples of organisms include humans, other mammals, bovines, rats, mice, dogs, monkeys, goat, sheep, cows, deer, and other non-mammalian animals. In certain embodiments, a subject is human. A human subject can be between the ages of about 1 year old to about 100 years old. In certain embodiments, subjects herein can be characterized by the disease being treated (e.g., a “AML subject”, a “cancer subject”, or a “leukemia subject”).

As used herein, the term “pediatric patient” refers to a patient 21 years or younger, in certain embodiments, a patient 18 years or younger, in certain embodiments, a patient 16 years or younger, in certain embodiments, a patient 14 years or younger, in certain embodiments, a patient 12 years or younger, in certain embodiments, a patient 10 years or younger, or in certain embodiments, a patient 8 years or younger.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous forms include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 2θ while still describing the particular XRPD peak.

Unless otherwise specified, to the extent that there is a discrepancy between a depicted chemical structure of a compound provided herein and a chemical name of a compound provided herein, the chemical structure shall control.

Compound

In certain embodiments, provided herein are co-crystals comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol having the following formula:

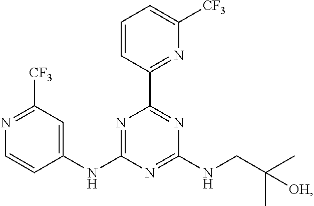 [SMILES: CC(C)(O)CNc1nc(Nc2ccnc(C(F)(F)F)c2)nc(-c2cccc(C(F)(F)F)n2)n1]

or a solvate, hydrate, stereoisomer, prodrug, or clathrate thereof (collectively referred to as Compound 1), and a coformer. The coformer can be any pharmaceutically acceptable coformer known in the art. In one embodiment, the coformer is fumaric acid, succinic acid, nicotinamide, benzoic acid, uracil, saccharin, citric acid, lactamide, or 4-hydroxybenzamide.

In certain embodiments, provided herein is a co-crystal comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol (Compound 1A), and a coformer.

In one embodiment, Compound 1A used in preparing the co-crystals provided herein is a crystalline solid. In one embodiment, Compound 1A comprises a mixture of polymorph forms of Compound 1A. In one embodiment, Compound 1A comprises a mixture of two polymorph forms of Compound 1A. In one embodiment, Compound 1 comprises a mixture of polymorph Form 17 and polymorph Form A of Compound 1A.

In one embodiment, Compound 1, for example, Compound 1A, can be synthesized using methods described in U.S. Pat. Nos. 9,512,107; 9,656,999; 9,732,062; 9,738,625; 9,751,863 and U.S. Publication No. 2017/0305885 A1, and PCT Publication No. WO 2016/126798, all of which are incorporated herein in their entireties.

The polymorphic forms of Compound 1A, including Form 1, Form 2, Form 17 and Form 19, are described in U.S. Pat. No. 9,738,625 and WO 2016/126798, the entirety of each of which is incorporated herein by reference. Polymorph Form A of Compound 1A is described herein.

### Form A of Compound 1A

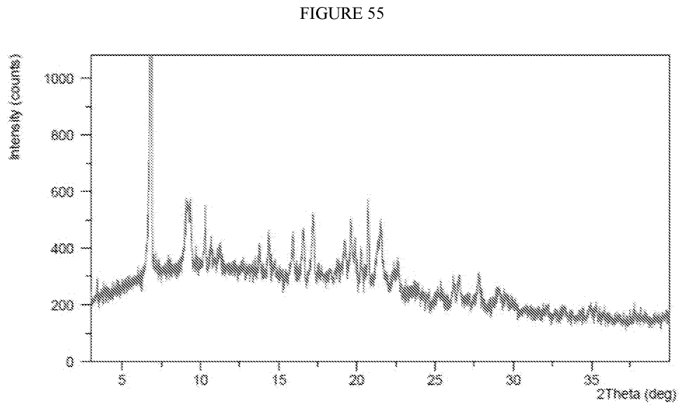

In one embodiment, a single crystalline form, Form A, of Compound 1A is characterized by the XRPD pattern shown in FIG. 55 obtained using CuKα radiation.

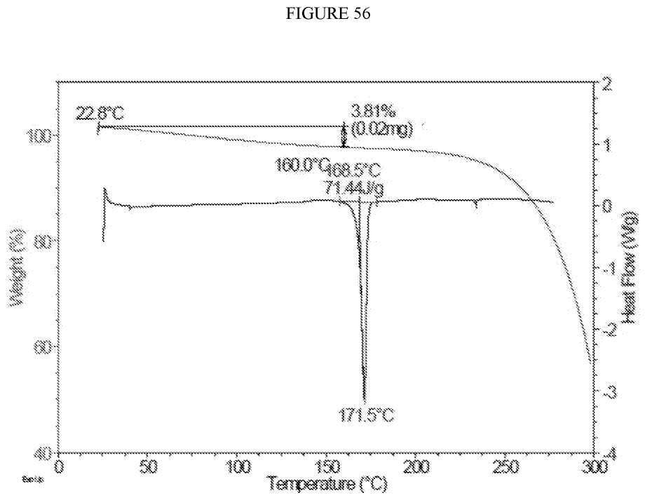

In another embodiment, Form A is characterized by the DSC shown in FIG. 56. The DSC graph plots the heat flow as a function of temperature from a sample, the temperature rate change being about 10° C./min. The profile is characterized by one endotherm at 168.5° C. (onset temperature).

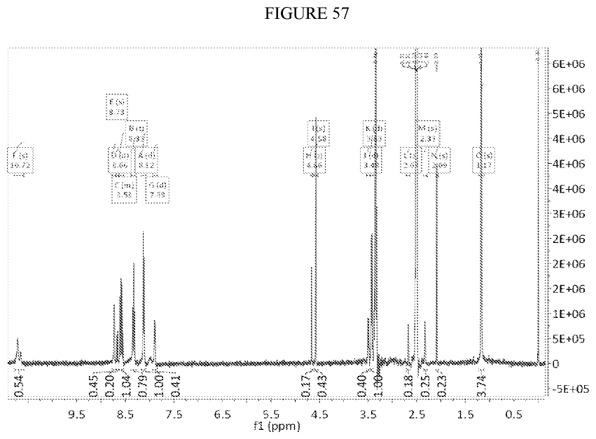

In another embodiment, Form A is characterized by TGA profile shown in FIG. 56. The TGA profile graphs the percent loss of weight of the sample as a function of temperature, the temperature rate change being about 10° C./min. The weight loss represents a loss of about 3.8% of the weight of the sample as the temperature is increased to about 160.0° C. FIG. 57 provides the ¹H NMR spectrum, which indicates that the molar ratio of acetone to Form A is 0.06 due to the existence of residual solvent.

### Form G of Compound 1A

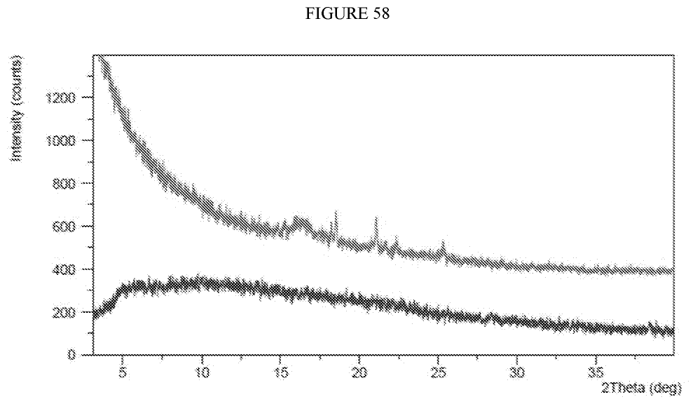

In one embodiment, a single crystalline form, Form G, of Compound 1A is characterized by the XRPD pattern shown in FIG. 58 obtained using CuKα radiation.

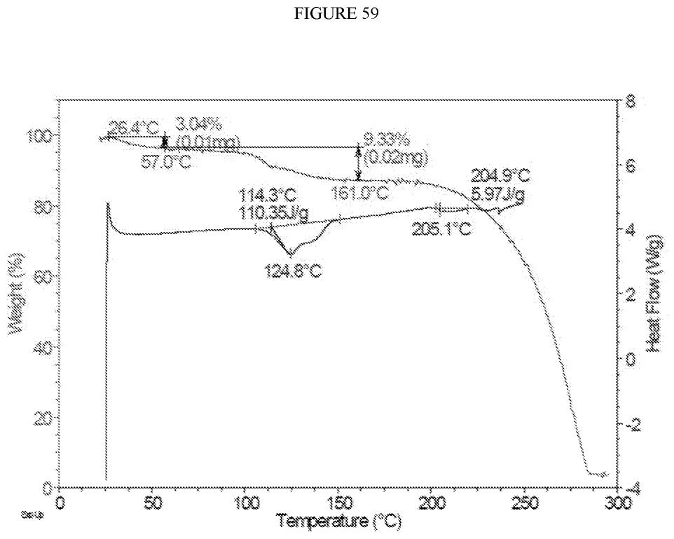

In another embodiment, Form G is characterized by the DSC shown in FIG. 59. The DSC graph plots the heat flow as a function of temperature from a sample, the temperature rate change being about 10° C./min. The profile is characterized by two endotherms at 114.3° C. and 204.9° C. (onset temperature).

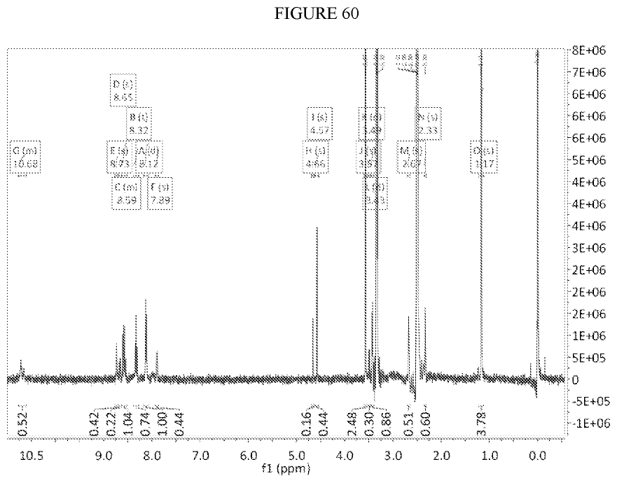

In another embodiment, Form A is characterized by the TGA profile shown in FIG. 59. The TGA profile graphs the percent loss of weight of the sample as a function of temperature, the temperature rate change being about 10° C./min. The weight loss represents a loss of about 12.4% of the weight of the sample as the temperature is increased to about 160.0° C. When heated to 160° C., Type G converted to amorphous, as shown in FIG. 59. FIG. 60 provides the ¹H NMR spectrum, which indicates that the molar ratio of dioxane to Form A is 0.5.

### Form K of Compound 1A

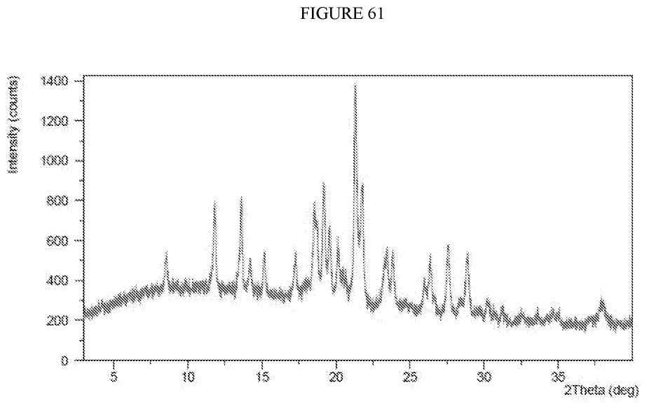

In one embodiment, a single crystalline form, Form K, of Compound 1A is characterized by the XRPD pattern shown in FIG. 61 obtained using CuKα radiation.

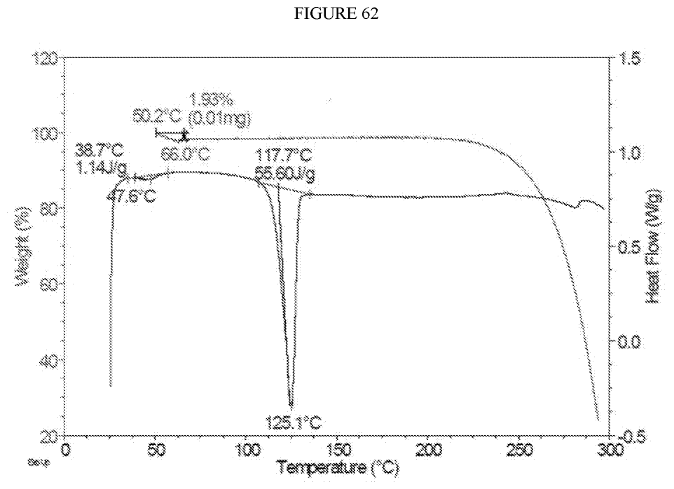

In another embodiment, Form K is characterized by the DSC profile shown in FIG. 62. The DSC graph plots the heat flow as a function of temperature from a sample, the temperature rate change being about 10° C./min. The profile is characterized by two endotherms at 38.7° C. and 117.7° C. (onset temperature).

In another embodiment, Form K is characterized by TGA profile shown in FIG. 62. The TGA profile graphs the percent loss of weight of the sample as a function of temperature, the temperature rate change being about 10° C./min. In one embodiment, Form K shows a weight loss of ˜1.9% when temperature is raised up to 66° C.

### Form FB3 of Compound 1A

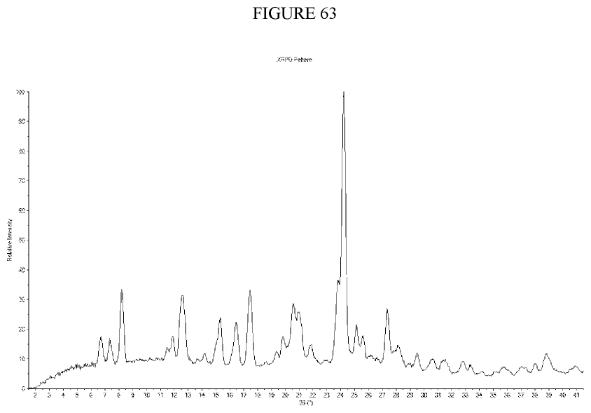

Form FB3 of Compound 1A comprises mainly Form 19 of Compound 1A. Form 19 of Compound 1A is described in U.S. Pat. No. 9,738,625. In one embodiment, Form FB3, of Compound 1A is characterized by the XRPD pattern shown in FIG. 63 obtained using CuKα radiation. In one embodiment, Form FB3 is characterized by an XRPD pattern having peaks at 20 angles of 8.22, 12.58 15.30, 16.46, 17.46, 24.22, 25.14 and 25.14°±0.2° obtained using CuKα radiation.

### Form FB7 of Compound 1A

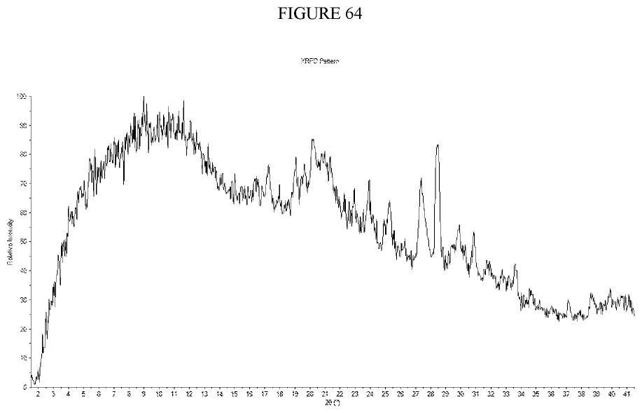

Form FB7 of Compound 1A is a mixture of Form K, Form 19 and Form 2 of Compound 1A. Forms 2 and 19 of Compound 1A are described in U.S. Pat. No. 9,738,625. In one embodiment, Form FB7, of Compound 1A is characterized by the XRPD pattern shown in FIG. 64 obtained using CuKα radiation. In one embodiment, Form FB7 is characterized by an XRPD pattern having peaks at 20 angles of 17.22, 19.06, 20.22, 21.34, 22.26, 22.98, 23.90, 25.26, 27.38, 28.42, 29.86, 30.86, 33.62, 37.18, 38.70, and 39.82°+0.2° obtained using CuKα radiation.

Co-crystals

In one embodiment, provided herein is a crystal form comprising (a) Compound 1; and (b) a coformer. In one embodiment, provided herein is a crystal form comprising (a) Compound 1A; and (b) a coformer. In one embodiment, provided herein is a co-crystal comprising (a) Compound 1; and (b) a coformer. In one embodiment, provided herein is a co-crystal comprising (a) Compound 1A; and (b) a coformer.

In one embodiment, provided herein is an unsolvated co-crystal comprising (a) Compound 1A and (b) a coformer. In one embodiment, provided herein is an anhydrous co-crystal comprising (a) Compound 1A and (b) a coformer. In one embodiment, provided herein is an unsolvated crystal form comprising (a) Compound 1A and (b) a coformer. In one embodiment, provided herein is an anhydrous crystal form comprising (a) Compound 1A and (b) a coformer.

In one embodiment, provided herein is a solvated co-crystal comprising (a) Compound 1A and (b) a coformer. In one embodiment, provided herein is a hydrated co-crystal comprising (a) Compound 1A and (b) a coformer (e.g., a hydrate having a stoichiometric or non-stoichiometric amount of water). In one embodiment, provided herein is a hydrated form of (a) Compound 1A and (b) a coformer, including, but not limited to, a hemihydrate, a monohydrate, a dihydrate, a trihydrate, and the like. In one embodiment, the hydrated form is substantially crystalline. In one embodiment, the hydrated form is substantially amorphous. In one embodiment, the anhydrous form is substantially crystalline. In one embodiment, the anhydrous form is substantially amorphous. In one embodiment, provided herein is an unsolvated co-crystal comprising (a) Compound 1A and (b) a coformer. In one embodiment, provided herein is an anhydrous co-crystal comprising (a) Compound 1A and (b) a coformer. In one embodiment, provided herein is a hydrated co-crystal comprising (a) Compound 1A and (b) a coformer. In one embodiment, provided herein is a solvated co-crystal comprising (a) Compound 1A and (b) a coformer.

The co-crystals provided herein can be prepared by the methods described herein, or by techniques, including, but not limited to, heating, cooling, freeze drying, spray drying, lyophilization, quench cooling the melt, rapid solvent evaporation, slow solvent evaporation, solvent recrystallization, antisolvent addition, slurry recrystallization, crystallization from the melt, desolvation, recrystallization in confined spaces, such as, e.g., in nanopores or capillaries, recrystallization on surfaces or templates, such as, e.g., on polymers, recrystallization in the presence of additives, such as, e.g., co-crystal counter-molecules, desolvation, dehydration, rapid cooling, slow cooling, exposure to solvent and/or water, drying, including, e.g., vacuum drying, vapor diffusion, sublimation, grinding (including, e.g., cryo-grinding and solvent-drop grinding), microwave-induced precipitation, sonication-induced precipitation, laser-induced precipitation, and precipitation from a supercritical fluid. The particle size of the resulting co-crystals, which can vary (e.g., from nanometer dimensions to millimeter dimensions), can be controlled, e.g., by varying crystallization conditions, such as, e.g., the rate of crystallization and/or the crystallization solvent system, or by particle-size reduction techniques, e.g., grinding, milling, micronizing, or sonication.

In some embodiments, the co-crystal comprising (a) Compound 1 and (b) a coformer can be obtained by crystallization from certain solvent systems, for example, solvent systems comprising one or more of the following solvents: tetrahydrofuran (THF), acetonitrile, ethyl acetate, chloroform, acetone, 1,4-dioxane, ethanol, water and acetonitrile. In one embodiment, the solvent is selected from tetrahydrofuran, acetonitrile, ethyl acetate, chloroform, ethyl acetate, acetone/water, THF/water, 1,4-dioxane, ethanol/water and acetonitrile. Other examples of solvent systems are provided herein elsewhere. In certain embodiments, a co-crystal provided herein (e.g., a co-crystal comprising (a) Compound 1A and (b) a coformer) can be obtained by slurry crystallization, evaporation crystallization, cooling crystallization, precipitation crystallization, saturated (API) solution co-crystallization, slurry conversion, and wet co-grinding.

In certain embodiments, co-crystals can be prepared using solid-state methods such as solid-state grinding and solvent-drop grinding. In certain embodiments, co-crystals can be prepared using high-throughput screening. In certain embodiments co-crystals can be prepared using solution-based crystallization.

In certain embodiments, slurry crystallization is effected by adding solvent or solvent mixtures to a solid substrate, and the slurry is stirred, and optionally heated to various temperatures. In certain embodiments, the slurry is heated at about 25° C., about 50° C., about 80° C., or about 100° C. In certain embodiments, upon heating and cooling, the residual solvents of the slurry can be removed by wicking, or other suitable methods, such as filtration, centrifugation, or decantation, and the crystals can be dried in air or under vacuum.

In certain embodiments, evaporation crystallization is effected by adding a solvent or solvent mixture to a solid substrate, and allowing the solvent or solvent mixture to evaporate under ambient conditions. In certain embodiments, the residual solvent can be removed by wicking, or other suitable methods, such as filtration, centrifugation, or decantation, and the crystals can be dried in air or under vacuum.

In certain embodiments, precipitation crystallization is effected by adding a solvent or solvent mixture to a solid substrate, and subsequently adding an anti-solvent. In certain embodiments, the resultant mixture stands for a period of time, e.g., overnight, and under certain conditions, for example at room temperature. In certain embodiments, the residual solvent can be removed by wicking, or other suitable methods, such as filtration, centrifugation, or decantation, and the crystals can be dried in air or under vacuum.

In certain embodiments, cooling crystallization is effected by adding a solvent or solvent mixture to a solid substrate at elevated temperature, and allowing the resultant mixture to stand for a period of time at a reduced temperature. In certain embodiments, the elevated temperature is, for example, about 30° C., about 40° C., about 50° C., about 60° C., about 70° C., or about 80° C. In certain embodiments, the reduced temperature is, for example, about 15° C., about 10° C., about 5° C., about 0° C., about −5° C., about −10° C., about −15° C., or about −20° C. The residual solvent can be removed by wicking, or other suitable methods, such as filtration, centrifugation, or decantation, and the crystals can be dried in air or under vacuum.

In certain embodiments, saturated API solution co-crystallization is effected by adding the coformer to a saturated solution of the API, stirring the mixture for a period of time at ambient temperature. In one embodiment, the API is Compound 1. In one embodiment, the API is Compound 1A.

In certain embodiments, the wet co-grinding is effected by grinding a mixture of the API and coformer in a small amount of solvent. In one embodiment, the API is Compound 1. In one embodiment, the API is Compound 1A.

In certain embodiments, the non-covalent forces are one or more hydrogen bonds (H-bonds). The coformer may be H-bonded directly to the API or may be H-bonded to an additional molecule which is bound to the API. The additional molecule may be H-bonded to the API or bound ionically or covalently to the API. The additional molecule could also be a different API. In certain embodiments, the co-crystals may include one or more solvate molecules in the crystalline lattice, i.e., solvates of co-crystals, or a co-crystal further comprising a solvent or compound that is a liquid at room temperature. In certain embodiments, the non-covalent forces are pi-stacking, guest-host complexation and/or van der Waals interactions. Hydrogen bonding can result in several different intermolecular configurations. For example, hydrogen bonds can result in the formation of dimers, linear chains, or cyclic structures. These configurations can further include extended (two-dimensional) hydrogen bond networks and isolated triads.

The ratio of API to coformer may be stoichiometric or non-stoichiometric. In one embodiment, the ratio of API to coformer is about 5:1, 4:1, 3:1, 2.5:1, 2:1, 1.5:1, 1:1, 1:1.5, 1:2, 1:2.5, 1:3, 1:4, or 1:5. In one embodiment, the ratio of API to coformer is about 1:1. In one embodiment, the co-crystal comprises more than one coformers. In one embodiment, the co-crystal comprises two coformers. In one embodiment, the API is Compound 1. In one embodiment, the API is Compound 1A.

In another embodiment, provided herein are compositions comprising one or more co-crystal(s) comprising (a) Compound 1; and (b) a coformer. Also provided herein are compositions comprising: (i) one or more co-crystal(s) provided herein, and (ii) other active ingredient(s).

While not intending to be bound by any particular theory, certain co-crystals provided herein exhibit physical properties, e.g., solubility, dissolution rate, bioavailability, physical stability, chemical stability, flowability, fractability, or compressibility, appropriate for use in clinical and therapeutic dosage forms. In certain embodiments, a given API may form different co-crystals with many different counter-molecules, and some of these co-crystals may exhibit enhanced solubility or stability. In certain embodiments, pharmaceutical co-crystals increase the bioavailability or stability profile of a compound without the need for chemical (covalent) modification of the API.

Co-Crystal Comprising Compound 1A and Fumaric Acid

Certain embodiments herein provide co-crystals comprising Compound 1A and fumaric acid. In one embodiment, provided herein is a co-crystal comprising Compound 1A and fumaric acid that is substantially crystalline. In one embodiment, provided herein is a co-crystal comprising Compound 1A and fumaric acid. In one embodiment, provided herein is a hemi co-crystal comprising Compound 1A and fumaric acid.

In some embodiments, the co-crystal comprising compound 1A and fumaric acid provided herein is Form Fum1.

In some embodiments, Form Fum1 is obtained by co-melting a mixture of Compound 1A and fumaric acid in a ratio of 1:0.5.

In some embodiments, Form Fum1 is obtained by adding an aqueous solution of fumaric acid to a solution of Compound 1A in tetrahydrofuran, freeze drying the solution, adding tetrahydrofuran to the freeze dried mixture to obtain a paste, and sonicating the paste to obtain a product, and optionally, subjecting the product to an aging condition. In one embodiment, the aging condition a temperature of about 40° C. and 75% RH for about 2 days.

In some embodiments, Form Fum1 is obtained adding fumaric acid to a solvent system saturated with Compound 1A, stirring the mixture (e.g., at ambient temperature overnight), removing the solid phase, and then slowly evaporating the mother liquid. In one embodiment, Form Fum1 is obtained from the removed solid phase, optionally after subjecting to an aging condition. In one embodiment, Form Fum1 is obtained by evaporation of the mother liquid, optionally after subjecting to an aging condition. In one embodiment, the aging condition a temperature of about 40° C. and 75% RH for about 2 days. In one embodiment, the solvent saturated with Compound 1A is ethyl acetate. In one embodiment, the solvent saturated with Compound 1A is acetonitrile. In one embodiment, the solvent system saturated with Compound 1A is tetrahydrofuran and water mixture. In one embodiment, the solvent system is a 85:15 (v/v) mixture of tetrahydrofuran and water.

In one embodiment, the molar ratio of Compound 1A to fumaric acid is from about 2:1 to about 1:2. In some embodiments, the molar ratio of Compound 1A to fumaric acid is about 1:0.5.

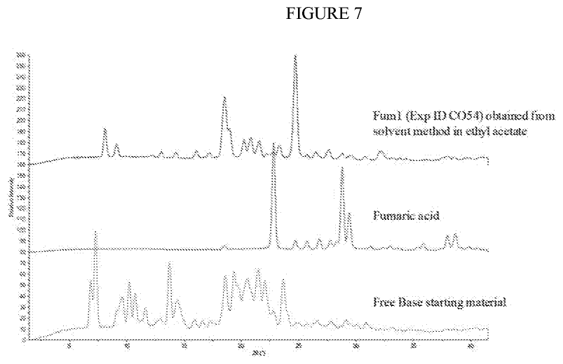

A representative overlay of XRPD patterns of Compound 1A, reference of fumaric acid, and Form Fum1 as obtained from the solvent mediated method in ethyl acetate is provided in FIG. 7.

In one embodiment, Form Fum1 is a non-solvated unhydrous form.

In some embodiments, Form Fum1 has a melting melting temperature of 212° C. as determined by DSC.

Co-Crystal Comprising Compound 1A and Succinic Acid

Certain embodiments herein provide co-crystals comprising Compound 1A and succinic acid. In one embodiment, provided herein is a co-crystal comprising Compound 1A and succinic acid that is substantially crystalline. In one embodiment, provided herein is a co-crystal comprising Compound 1A and succinic acid.

In some embodiments, the co-crystal comprising compound 1A and succinic acid provided herein is Form Suc1.

In some embodiments, Form Suc1 is obtained adding accinic acid to a solvent system saturated with Compound 1A, stirring the mixture (e.g., at ambient temperature overnight), removing the solid phase, and then slowly evaporating the mother liquid. In one embodiment, Form Suc1 is obtained from the removed solid phase, optionally after subjecting to an aging condition. In one embodiment, Form Suc1 is obtained by evaporation of the mother liquid, optionally after subjecting to an aging condition. In one embodiment, the aging condition a temperature of about 40° C. and 75% RH for about 2 days. In one embodiment, the solvent saturated with Compound 1A is acetonitrile.

In one embodiment, the molar ratio of Compound 1A to succinic acid is from about 2:1 to about 1:3. In some embodiments, the molar ratio of Compound 1A to succinic acid is about 1:0.5.

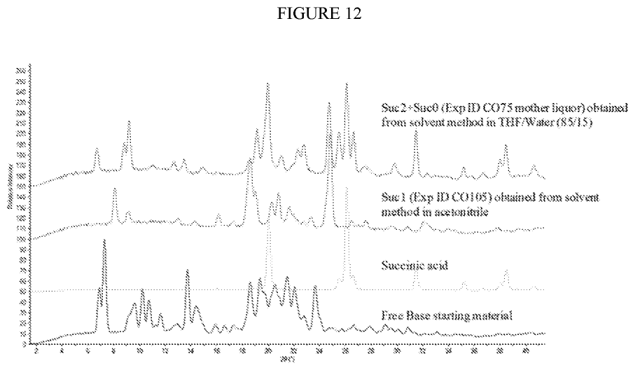

A representative overlay of XRPD patterns of Compound 1A, reference of succinic acid, Form Suc1 and a mixture of Form Suc2+Suc0 is provided in FIG. 12.

In one embodiment, Form Suc1 is a non-solvated unhydrous form.

In some embodiments, Form Suc1 has two endothermic events, at 156.8° C. and 179.8° C., as determined by DSC.

Co-Crystal Comprising Compound 1A and Nicotinamide

Certain embodiments herein provide co-crystals comprising Compound 1A and nicotinamide. In one embodiment, provided herein is a co-crystal comprising Compound 1A and nicotinamide that is substantially crystalline. In one embodiment, provided herein is a co-crystal comprising Compound 1A and nicotinamide.

In some embodiments, the co-crystal comprising compound 1A and nicotinamide provided herein is Form Nic1. In some embodiments, the co-crystal comprising compound 1A and nicotinamide provided herein is Form Nic2. In some embodiments, the co-crystal comprising compound 1A and nicotinamide provided herein is Form Nic3.

In some embodiments, Form Nic1 is obtained by co-melting a mixture of Compound 1A and nicotinamide in a ratio of 1:1.

In some embodiments, a co-crystal comprising Compound 1A and nicotinamide is obtained by adding an aqueous solution of nicotinamide to a solution of Compound 1A in tetrahydrofuran, freeze drying the solution, adding tetrahydrofuran to the freeze dried mixture to obtain a paste, and sonicating the paste to obtain a product, and optionally, subjecting the product to an aging condition. In one embodiment, the aging condition a temperature of about 40° C. and 75% RH for about 2 days. In some embodiments, the co-crystal obtained by this method comprises Form Nic3.

In some embodiments, Form Nic1 is obtained adding nicotinamide to a solvent saturated with Compound 1A, stirring the mixture (e.g., at ambient temperature overnight), removing the solid phase, and then slowly evaporating the mother liquid. In one embodiment, Form Nic1 and Nic 3 are obtained from the removed solid phase, optionally after subjecting to an aging condition. In one embodiment, the aging condition a temperature of about 40° C. and 75% RH for about 2 days. In one embodiment, the solvent saturated with Compound 1A is ethyl acetate. In one embodiment, the solvent saturated with Compound 1A is 1,4-dioxane. In one embodiment, Form Nic1 is obtained when the solvent is ethyl acetate. In one embodiment, Form Nic3 is obtained when the solvent is 1,4-dioxane.

In one embodiment, the molar ratio of Compound 1A to nicotinamide is from about 2:1 to about 1:2. In some embodiments, the molar ratio of Compound 1A to nicotinamide is about 1:1. In some embodiments, the molar ratio of Compound 1A to nicotinamide is about 1:0.8.

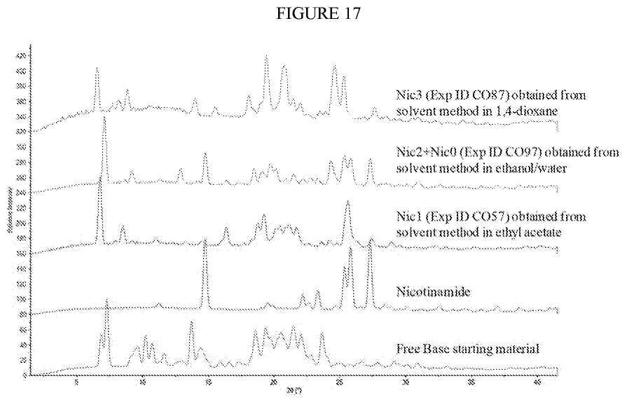

A representative overlay of XRPD patterns of Compound 1A, reference of nicotinamide, Form Nic1, Forms Nic2+Nic0 and Form Nic3 as obtained from the solvent mediated method in ethyl acetate is provided in FIG. 17.

In one embodiment, Form Nic1 is a non-solvated unhydrous form. In one embodiment, Form Nic3 is a solvated form. In one embodiment, Form Nic13 converted to Nic1 after desolvation.

In some embodiments, Form Nic1 has a melting melting temperature of 187° C. as determined by DSC.

Co-Crystal Comprising Compound 1A and Benzoic Acid

Certain embodiments herein provide co-crystals comprising Compound 1A and benzoic acid. In one embodiment, provided herein is a co-crystal comprising Compound 1A and benzoic acid that is substantially crystalline. In one embodiment, provided herein is a co-crystal comprising Compound 1A and benzoic acid.

In some embodiments, the co-crystal comprising compound 1A and benzoic acid provided herein is Form Ben1.

In some embodiments, Form Ben1 is obtained by co-melting a mixture of Compound 1A and benzoic acid in a ratio of 1:1.

In some embodiments, a co-crystal comprising Compound 1A and benzoic acid is obtained by adding an aqueous solution of benzoic acid to a solution of Compound 1A in solvent, and freeze drying the solution. In one embodiment, a solvent is added to the freeze dried mixture to obtain a paste, and the paste is sonicated to obtain a product, and optionally, the product is subjected to an aging condition. In one embodiment, the solvent is tetrahydrofuran, acetonitrile, ethyl acetate or chloroform. In one embodiment, the aging condition a temperature of about 40° C. and 75% RH for about 2 days. In some embodiments, the co-crystal obtained by this method comprises Form Ben1.

In one embodiment, the molar ratio of Compound 1A to benzoic acid is from about 2:1 to about 1:2. In some embodiments, the molar ratio of Compound 1A to benzoic acid is about 1:1.

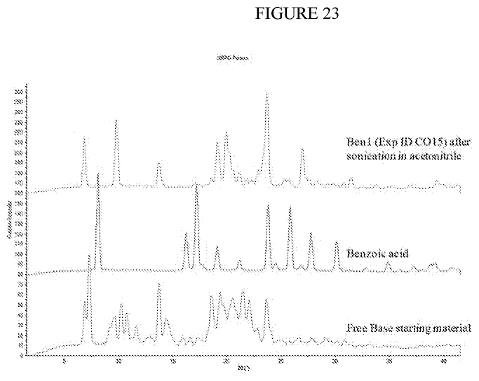

A representative overlay of XRPD patterns of Compound 1A, reference of benzoic acid, and Form Ben1 as obtained from the solvent mediated method in ethyl acetate is provided in FIG. 23.

In one embodiment, Form Ben1 is a non-solvated unhydrous form.

In some embodiments, Form Ben1 has a melting melting temperature of 151° C. as determined by DSC.

Co-Crystal Comprising Compound 1A and Uracil

Certain embodiments herein provide co-crystals comprising Compound 1A and uracil. In one embodiment, provided herein is a co-crystal comprising Compound 1A and uracil that is substantially crystalline. In one embodiment, provided herein is a co-crystal comprising Compound 1A and uracil.

In some embodiments, the co-crystal comprising Compound 1A and uracil provided herein is Form Ura1.

In some embodiments, Form Ura1 is obtained by co-melting a mixture of Compound 1A and benzoic acid in a ratio of 1:1.

In some embodiments, Form Ura1 is obtained adding uracil to a solvent saturated with Compound 1A, stirring the mixture (e.g., at ambient temperature overnight), and removing the solid phase. In one embodiment, Form Ura1 is obtained from the removed solid phase, optionally after subjecting to an aging condition. In one embodiment, the aging condition a temperature of about 40° C. and 75% RH for about 2 days. In one embodiment, the solvent saturated with Compound 1A is acetonitrile.

In one embodiment, the molar ratio of Compound 1A to uracil is from about 2:1 to about 1:2. In some embodiments, the molar ratio of Compound 1A to uracil is about 1:1.

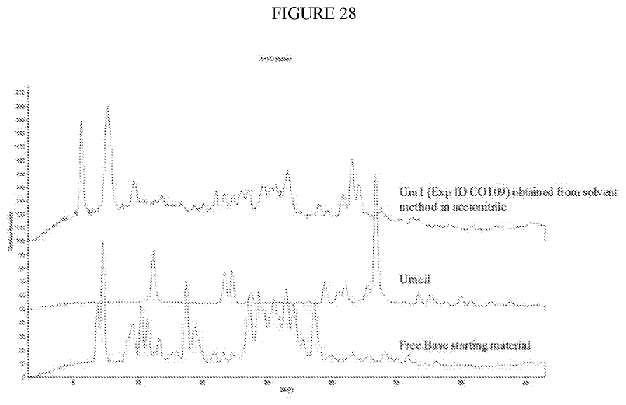

A representative overlay of XRPD patterns of Compound 1A, reference of uracil, and Form Ura1 is provided in FIG. 28.

In some embodiments, Form Ura1 has two endothermic events, at 187° C. and 197° C., as determined by DSC.

Co-Crystal Comprising Compound 1A and Saccharin

Certain embodiments herein provide co-crystals comprising Compound 1A and saccharin. In one embodiment, provided herein is a co-crystal comprising Compound 1A and saccharin that is substantially crystalline. In one embodiment, provided herein is a co-crystal comprising Compound 1A and saccharin.

In some embodiments, the co-crystal comprising compound 1A and saccharin provided herein is Form Sac1.

In some embodiments, a co-crystal comprising Compound 1A and saccharin is obtained by adding an aqueous solution of saccharin to a solution of Compound 1A in tetrahydrofuran, freeze drying the solution, and optionally subjecting the product to an aging condition. In one embodiment, the aging condition a temperature of about 40° C. and 75% RH for about 2 days. In some embodiments, the co-crystal obtained by this method comprises Form Sac3.

In some embodiments, Form Sac1 is obtained adding saccharin to a solvent saturated with Compound 1A, stirring the mixture (e.g., at ambient temperature overnight), removing the solid phase, and then slowly evaporating the mother liquid. In one embodiment, Form Sac1 isobtained from the removed solid phase, optionally after subjecting to an aging condition. In one embodiment, Form Sac1 isobtained after evaporating the mother liquid, optionally after subjecting to an aging condition. In one embodiment, the aging condition a temperature of about 40° C. and 75% RH for about 2 days. In one embodiment, the solvent saturated with Compound 1A is ethyl acetate. In one embodiment, the solvent saturated with Compound 1A is a mixture of acetone and water. In one embodiment, the solvent saturated with Compound 1A is a mixture of 90:10 (v/v) acetone and water. In one embodiment, the solvent saturated with Compound 1A is a mixture of ethanol and water. In one embodiment, the solvent saturated with Compound 1A is a mixture of 83:17 (v/v) ethanol and water.

In one embodiment, the molar ratio of Compound 1A to saccharin is from about 2:1 to about 1:2. In some embodiments, the molar ratio of Compound 1A to saccharin is about 1:2.

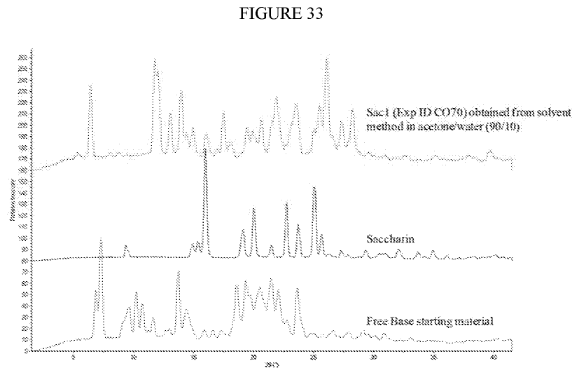

A representative overlay of XRPD patterns of Compound 1A, reference of saccharin, and Form Sac1 is provided in FIG. 33.

In one embodiment, Form sac is a non-solvated unhydrous form.

In some embodiments, Form Nic1 has a melting melting temperature of 172° C. as determined by DSC.

Co-crystal Comprising Compound 1A and Citric acid

Certain embodiments herein provide co-crystals comprising Compound 1A and citric acid. In one embodiment, provided herein is a co-crystal comprising Compound 1A and citric acid that is substantially crystalline. In one embodiment, provided herein is a co-crystal comprising Compound 1A and citric acid.

In some embodiments, the co-crystal comprising compound 1A and citric acid provided herein is Form Cit1. In some embodiments, the co-crystal comprising compound 1A and citric acid provided herein is Form Cit2. In some embodiments, the co-crystal comprising compound 1A and citric acid provided herein is Form Cit3. In some embodiments, the co-crystal comprising compound 1A and citric acid provided herein is Form Cit4.

In some embodiments, a co-crystal comprising Compound 1A and citric acid is obtained by adding an aqueous solution of citric acid to a solution of Compound 1A in tetrahydrofuran, freeze drying the solution, adding tetrahydrofuran to the freeze dried mixture to obtain a paste, and sonicating the paste to obtain a product, and optionally, subjecting the product to an aging condition. In one embodiment, the aging condition a temperature of about 40° C. and 75% RH for about 2 days. In some embodiments, the co-crystal obtained by this method comprises Form Cit1. In some embodiments, the co-crystal obtained by this method comprises Form Cit2. In some embodiments, the co-crystal obtained by this method comprises Form Cit4.

In some embodiments, Form Cit 3 and/or Form Cit 4 are obtained adding citric acid to a solvent saturated with Compound 1A, stirring the mixture (e.g., at ambient temperature overnight), removing the solid phase. In one embodiment, Form Nic13 and/or Form 4 are obtained from the removed solid phase, optionally after subjecting to an aging condition. In one embodiment, the aging condition a temperature of about 40° C. and 75% RH for about 2 days. In one embodiment, the solvent saturated with Compound 1A is ethyl acetate. In one embodiment, the solvent saturated with Compound 1A is a mixture of ethanol and water. In one embodiment, the solvent saturated with Compound 1A is a mixture of 83:17 (v/v) ethanol and water. In one embodiment, the solvent saturated with Compound 1A is acetonitril. In one embodiment, Form Cit3 is obtained when the solvent is ethyl acetate. In one embodiment, Form Cit4 is obtained when the solvent is a mixture of 83:17 (v/v) ethanol and water. In one embodiment, Form Cit4 is obtained when the solvent is acetonitrile.

In one embodiment, the molar ratio of Compound 1A to citric acid is from about 2:1 to about 1:2. In some embodiments, the molar ratio of Compound 1A to citric acid is about 1:1. In some embodiments, the molar ratio of Compound 1A to citric acid is about 1:1.4. In some embodiments, the molar ratio of Compound 1A to citric acid is about 1:0.9

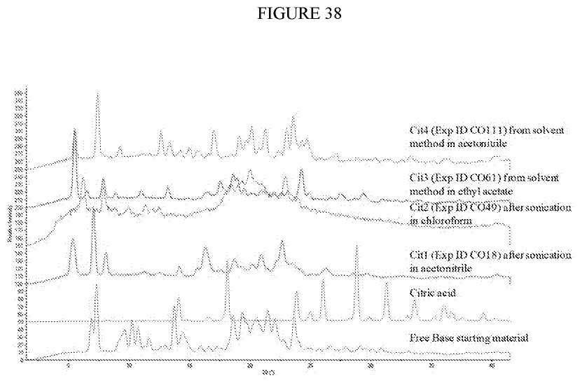

A representative overlay of XRPD patterns of Compound 1A, reference of citric acid, and Forms Cit1, Cit2, Cit3 and Cit4 is provided in FIG. 38.

In one embodiment, Form Cit3 is a non-solvated unhydrous form.

In some embodiments, Form Cit3 has a melting melting temperature of 180° C. as determined by DSC.

In one embodiment, Form Cit4 is a non-solvated unhydrous form.

In some embodiments, Form Cit4 has a melting melting temperature of 187° C., and another endothermic event at 151° C. as determined by DSC.

Co-Crystal Comprising Compound 1A and Lactamide

Certain embodiments herein provide co-crystals comprising Compound 1A and lactamide. In one embodiment, provided herein is a co-crystal comprising Compound 1A and lactamide that is substantially crystalline. In one embodiment, provided herein is a co-crystal comprising Compound 1A and lactamide. In one embodiment, provided herein is a hemi co-crystal comprising Compound 1A and lactamide.

In some embodiments, the co-crystal comprising compound 1A and lactamide provided herein is Form Lac1.

In some embodiments, Form Lac is obtained by co-melting a mixture of Compound 1A and lactamide in a ratio of 1:1.

A representative overlay of XRPD patterns of Compound 1A, reference of lactamide, Form Lac1, Form Lac2, and a mixture of Forms Lac3+Lac0 as obtained from the solvent mediated method in ethyl acetate is provided in FIG. 7.

In one embodiment, Form Lac is a non-solvated unhydrous form.

In some embodiments, Form Fum1 has a melting melting temperature of 138° C. as determined by DSC.

Co-Crystal Comprising Compound 1A and 4-Hydroxybenzamide

Certain embodiments herein provide co-crystals comprising Compound 1A and 4-hydroxybenzamide. In one embodiment, provided herein is a co-crystal comprising Compound 1A and 4-hydroxybenzamide that is substantially crystalline. In one embodiment, provided herein is a co-crystal comprising Compound 1A and 4-hydroxybenzamide. In one embodiment, provided herein is a hemi co-crystal comprising Compound 1A and 4-hydroxybenzamide.

In some embodiments, the co-crystal comprising compound 1A and 4-hydroxybenzamide provided herein is Form Hbe1.

In some embodiments, Form Hbe is obtained by co-melting a mixture of Compound 1A and 4-hydroxybenzamide in a ratio of 1:1.

In some embodiments, Form Hbe1 is obtained adding 4-hydroxybenzamide to a solvent system saturated with Compound 1A, stirring the mixture (e.g., at ambient temperature overnight), removing the solid phase, and then slowly evaporating the mother liquid. In one embodiment, Form Hbe1 is obtained from the removed solid phase, optionally after subjecting to an aging condition. In one embodiment, the aging condition a temperature of about 40° C. and 75% RH for about 2 days. In one embodiment, the solvent saturated with Compound 1A is ethyl acetate. In one embodiment, the solvent saturated with Compound 1A is acetonitrile.

In one embodiment, the molar ratio of Compound 1A to 4-hydroxybenzamide is from about 2:1 to about 1:2. In some embodiments, the molar ratio of Compound 1A to 4-hydroxybenzamide is about 1:1.

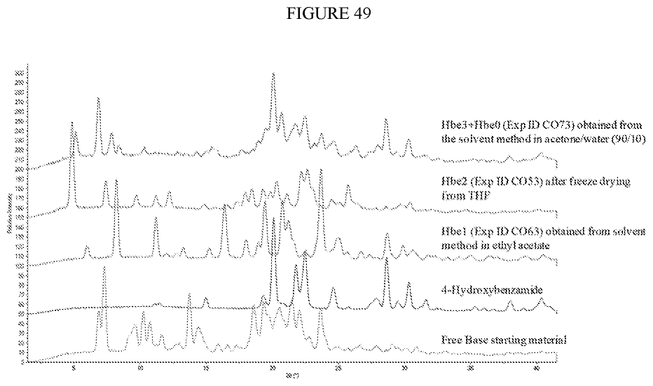

A representative overlay of XRPD patterns of Compound 1A, reference of 4 hydroxybenzamide, Form Hbe1, Form Hbe2 and Forms Hbe3+Hbe0 is provided in FIG. 49.

In some embodiments, Form Hbe1 has a melting melting temperature of 176° C. as determined by DSC.

Compositions Containing the Co-Crystals and Routes of Administration

In one embodiment, the co-crystals provided herein are formulated with a pharmaceutically acceptable carrier or adjuvant into pharmaceutically acceptable compositions prior to be administered to a subject. In another embodiment, such pharmaceutically acceptable compositions further comprise additional therapeutic agents in amounts effective for achieving a modulation of disease or disease symptoms, including those described herein.

Pharmaceutically acceptable carriers, adjuvants and vehicles that may be used in the pharmaceutical compositions of one aspect of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as TWEENs or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of the co-crystals described herein.

In one embodiment, the pharmaceutical composition comprises a co-crystal and an excipient. In one embodiment, the pharmaceutical composition that comprises a co-crystal and an excipient, is for oral administration. In one embodiment, the excipient is a diluent, a binder, a disintegrant, a wetting agent, a stabilizer, a glidant, or a lubricant.

In one embodiment, the diluent is a microcrystalline cellulose.

In one embodiment, the binder is a hydroxypropyl cellulose.

In one embodiment, the disintegrant is sodium starch glycolate.

In one embodiment, the wetting agent is sodium lauryl sulfate.

In one embodiment, the stabilizer is hypromellose acetate succinate.

In one embodiment, the glidant is colloidal silicon dioxide.

In one embodiment, the lubricant is magnesium stearate.

Oral delivery formats include, but are not limited to, tablets, capsules, caplets, solutions, suspensions, and syrups, and may also comprise a plurality of granules, beads, powders or pellets that may or may not be encapsulated. Such formats may also be referred to herein as the “drug core” which contains a co-crystal provided herein.

Particular embodiments herein provide solid oral dosage forms that are tablets or capsules. In certain embodiments, the formulation is a tablet comprising a co-crystal provided herein. In certain embodiments, the formulation is a capsule comprising a co-crystal provided herein. In certain embodiments, the tablets or capsules provided herein optionally comprise one or more excipients, such as, for example, glidants, diluents, lubricants, colorants, disintegrants, granulating agents, binding agents, polymers, and coating agents. In certain embodiments, the formulation is an immediate release tablet. In certain embodiments, the formulation is a controlled release tablet releasing the active pharmaceutical ingredient (API), e.g., substantially in the stomach. In certain embodiments, the formulation is a hard gelatin capsule. In certain embodiments, the formulation is a soft gelatin capsule. In certain embodiments, the capsule is a hydroxypropyl methylcellulose (HPMC) capsule. In certain embodiments, the formulation is an immediate release capsule. In certain embodiments, the formulation is an immediate or controlled release capsule releasing the API, e.g., substantially in the stomach. In certain embodiments, the formulation is a rapidly disintegrating tablet that dissolves substantially in the mouth following administration.

Particular embodiments herein provide pharmaceutical formulations (e.g., immediate release oral formulations and/or formulations that release the API substantially in the stomach) comprising a co-crystal provided herein that achieve a particular AUC value (e.g., AUC(0−t) or AUC(0−∞)) in the subject (e.g., human) to which the formulation is orally administered. In one embodiment, the API is Compound 1. In one embodiment, the API is Compound 1A. Particular embodiments provide oral formulations that achieve an AUC value of at least about 25 ng-hr/mL, at least about 50 ng-hr/mL, at least about 75 ng-hr/mL, at least about 100 ng-hr/mL, at least about 150 ng-hr/mL, at least about 200 ng-hr/mL, at least about 250 ng-hr/mL, at least about 300 ng-hr/mL, at least about 350 ng-hr/mL, at least about 400 ng-hr/mL, at least about 450 ng-hr/mL, at least about 500 ng-hr/mL, at least about 550 ng-hr/mL, at least about 600 ng-hr/mL, at least about 650 ng-hr/mL, at least about 700 ng-hr/mL, at least about 750 ng-hr/mL, at least about 800 ng-hr/mL, at least about 850 ng-hr/mL, at least about 900 ng-hr/mL, at least about 950 ng-hr/mL, at least about 1000 ng-hr/mL, at least about 1100 ng-hr/mL, at least about 1200 ng-hr/mL, at least about 1300 ng-hr/mL, at least about 1400 ng-hr/mL, at least about 1500 ng-hr/mL, at least about 1600 ng-hr/mL, at least about 1700 ng-hr/mL, at least about 1800 ng-hr/mL, at least about 1900 ng-hr/mL, at least about 2000 ng-hr/mL, at least about 2250 ng-hr/mL, or at least about 2500 ng-hr/mL. In particular embodiments, the AUC determination is obtained from a time-concentration pharmacokinetic profile obtained from the blood samples of animals or human volunteers following dosing.

Particular embodiments herein provide pharmaceutical formulations (e.g., immediate release oral formulations and/or formulations that release the API substantially in the stomach) comprising a co-crystal provided herein that achieve a particular maximum plasma concentration (“Cmax”) in the subject to which the formulation is orally administered. In one embodiment, the API is Compound 1. In one embodiment, the API is Compound 1A. Particular embodiments provide oral formulations that achieve a Cmax of Compound 1 of at least about 25 ng/mL, at least about 50 ng/mL, at least about 75 ng/mL, at least about 100 ng/mL, at least about 150 ng/mL, at least about 200 ng/mL, at least about 250 ng/mL, at least about 300 ng/mL, at least about 350 ng/mL, at least about 400 ng/mL, at least about 450 ng/mL, at least about 500 ng/mL, at least about 550 ng/mL, at least about 600 ng/mL, at least about 650 ng/mL, at least about 700 ng/mL, at least about 750 ng/mL, at least about 800 ng/mL, at least about 850 ng/mL, at least about 900 ng/mL, at least about 950 ng/mL, at least about 1000 ng/mL, at least about 1100 ng/mL, at least about 1200 ng/mL, at least about 1300 ng/mL, at least about 1400 ng/mL, at least about 1500 ng/mL, at least about 1600 ng/mL, at least about 1700 ng/mL, at least about 1800 ng/mL, at least about 1900 ng/mL, at least about 2000 ng/mL, at least about 2250 ng/mL, or at least about 2500 ng/mL.

Particular embodiments herein provide pharmaceutical formulations (e.g., immediate release oral formulations and/or formulations that release the API substantially in the stomach) comprising a co-crystal provided herein that achieve a particular time to maximum plasma concentration (“Tmax”) in the subject to which the formulation is orally administered. In one embodiment, the API is Compound 1. In one embodiment, the API is Compound 1A. Particular embodiments provide oral formulations that achieve a Tmax of Compound 1 of less than about 10 min., less than about 15 min., less than about 20 min., less than about 25 min., less than about 30 min., less than about 35 min., less than about 40 min., less than about 45 min., less than about 50 min., less than about 55 min., less than about 60 min., less than about 65 min., less than about 70 min., less than about 75 min., less than about 80 min., less than about 85 min., less than about 90 min., less than about 95 min., less than about 100 min., less than about 105 min., less than about 110 min., less than about 115 min., less than about 120 min., less than about 130 min., less than about 140 min., less than about 150 min., less than about 160 min., less than about 170 min., less than about 180 min., less than about 190 min., less than about 200 min., less than about 210 min., less than about 220 min., less than about 230 min., or less than about 240 min. In particular embodiments, the Tmax value is measured from the time at which the formulation is orally administered.

Particular embodiments herein provide oral dosage forms comprising a co-crystal provided herein wherein the oral dosage forms have an enteric coating. Particular embodiments provide a permeable or partly permeable (e.g., “leaky”) enteric coating with pores. In particular embodiments, the permeable or partly permeable enteric-coated tablet releases Compound 1 in an immediate release manner substantially in the stomach.

Provided herein are dosage forms designed to maximize the absorption and/or efficacious delivery of Compound 1, upon oral administration, e.g., for release substantially in the stomach. Accordingly, certain embodiments herein provide a solid oral dosage form comprising a co-crystal provided herein using pharmaceutical excipients designed for immediate release of the API upon oral administration, e.g., substantially in the stomach. In one embodiment, the API is Compound 1. In one embodiment, the API is Compound 1A. Particular immediate release formulations comprise a specific amount of a co-crystal provided herein and optionally one or more excipients. In certain embodiments, the formulation may be an immediate release tablet or an immediate release capsule (such as, e.g., an HPMC capsule).

Provided herein are methods of making the formulations provided herein comprising a co-crystal provided herein provided herein (e.g., immediate release oral formulations and/or formulations that release the API substantially in the stomach). In one embodiment, the API is Compound 1. In one embodiment, the API is Compound 1A. In particular embodiments, the formulations provided herein may be prepared using conventional methods known to those skilled in the field of pharmaceutical formulation, as described, e.g., in pertinent textbooks. See, e.g., REMINGTON, THE SCIENCE AND PRACTICE OF PHARMACY, 20th Edition, Lippincott Williams & Wilkins, (2000); ANSEL et al., PHARMACEUTICAL DOSAGE FORMS AND DRUG DELIVERY SYSTEMS, 7th Edition, Lippincott Williams & Wilkins, (1999); GIBSON, PHARMACEUTICAL PREFORMULATION AND FORMULATION, CRC Press (2001).

In particular embodiments, formulations provided herein (e.g., immediate release oral formulations, formulations that release the API substantially in the stomach, or rapidly disintegrating formulations that dissolve substantially in the mouth) comprise a co-crystal provided herein in a specific amount. In one embodiment, the API is Compound 1. In one embodiment, the API is Compound 1A. In particular embodiments, the specific amount of a co-crystal provided in the formulation is, e.g., about 10 mg. In one embodiment, the specific amount is about 20 mg. In one embodiment, the specific amount is about 40 mg. In one embodiment, the specific amount is about 60 mg. In one embodiment, the specific amount is about 80 mg. In one embodiment, the specific amount is about 100 mg. In one embodiment, the specific amount is about 120 mg. In one embodiment, the specific amount is about 140 mg. In one embodiment, the specific amount is about 150 mg. In one embodiment, the specific amount is about 160 mg. In one embodiment, the specific amount is about 180 mg. In one embodiment, the specific amount is about 200 mg. In one embodiment, the specific amount is about 220 mg. In one embodiment, the specific amount is about 240 mg. In one embodiment, the specific amount is about 260 mg. In one embodiment, the specific amount is about 280 mg. In one embodiment, the specific amount is about 300 mg. In one embodiment, the specific amount is about 320 mg. In one embodiment, the specific amount is about 340 mg. In one embodiment, the specific amount is about 360 mg. In one embodiment, the specific amount is about 380 mg. In one embodiment, the specific amount is about 400 mg. In one embodiment, the specific amount is about 420 mg. In one embodiment, the specific amount is about 440 mg. In one embodiment, the specific amount is about 460 mg. In one embodiment, the specific amount is about 480 mg. In one embodiment, the specific amount is about 500 mg. In one embodiment, the specific amount is about 600 mg. In one embodiment, the specific amount is about 700 mg. In one embodiment, the specific amount is about 800 mg. In one embodiment, the specific amount is about 900 mg. In one embodiment, the specific amount is about 1000 mg. In one embodiment, the specific amount is about 1100 mg. In one embodiment, the specific amount is about 1200 mg. In one embodiment, the specific amount is about 1300 mg. In one embodiment, the specific amount is about 1400 mg. In one embodiment, the specific amount is about 1500 mg. In one embodiment, the specific amount is about 1600 mg. In one embodiment, the specific amount is about 1700 mg. In one embodiment, the specific amount is about 1800 mg. In one embodiment, the specific amount is about 1900 mg. In one embodiment, the specific amount is about 2000 mg. In one embodiment, the specific amount is about 2100 mg. In one embodiment, the specific amount is about 2200 mg. In one embodiment, the specific amount is about 2300 mg. In one embodiment, the specific amount is about 2400 mg. In one embodiment, the specific amount is about 2500 mg. In one embodiment, the specific amount is about 3000 mg. In one embodiment, the specific amount is about 4000 mg. In one embodiment, the specific amount is about 5000 mg.

In certain embodiments, the formulation is a tablet, wherein the tablet is manufactured using standard, art-recognized tablet processing procedures and equipment. In certain embodiments, the method for forming the tablets is direct compression of a powdered, crystalline and/or granular composition comprising a co-crystal provided herein alone or in combination with one or more excipients, such as, for example, carriers, additives, polymers, or the like. In certain embodiments, as an alternative to direct compression, the tablets may be prepared using wet granulation or dry granulation processes. In certain embodiments, the tablets are molded rather than compressed, starting with a moist or otherwise tractable material. In certain embodiments, compression and granulation techniques are used.

In certain embodiments, the formulation is a capsule, wherein the capsules may be manufactured using standard, art-recognized capsule processing procedures and equipments. In certain embodiments, soft gelatin capsules may be prepared in which the capsules contain a mixture of a co-crystal provided herein and vegetable oil or non-aqueous, water miscible materials such as, for example, polyethylene glycol and the like. In certain embodiments, hard gelatin capsules may be prepared containing granules of a co-crystal provided herein in combination with a solid pulverulent carrier, such as, for example, lactose, saccharose, sorbitol, mannitol, potato starch, corn starch, amylopectin, cellulose derivatives, or gelatin. In certain embodiments, a hard gelatin capsule shell may be prepared from a capsule composition comprising gelatin and a small amount of plasticizer such as glycerol. In certain embodiments, as an alternative to gelatin, the capsule shell may be made of a carbohydrate material. In certain embodiments, the capsule composition may additionally include polymers, colorings, flavorings and opacifiers as required. In certain embodiments, the capsule comprises HPMC.

In certain embodiments, the formulation of a co-crystal provided herein is prepared using aqueous solvents without causing significant hydrolytic degradation of the compound. In particular embodiments, the formulation of a co-crystal provided herein is a tablet which contains a coating applied to the drug core using aqueous solvents without causing significant hydrolytic degradation of the compound in the formulation. In certain embodiments, water is employed as the solvent for coating the drug core. In certain embodiments, the oral dosage form of a co-crystal provided herein is a tablet containing a film coat applied to the drug core using aqueous solvents. In particular embodiments, water is employed as the solvent for film-coating. In particular embodiments, the tablet containing a co-crystal provided herein is film-coated using aqueous solvents without effecting degradation of the pharmaceutical composition. In particular embodiments, water is used as the film coating solvent without effecting degradation of the pharmaceutical composition. In certain embodiments, an oral dosage form comprising a co-crystal provided herein and an aqueous film coating effects immediate drug release upon oral delivery. In certain embodiments, the oral dosage form comprising a co-crystal provided herein and an aqueous film coating effects controlled drug release to the upper gastrointestinal tract, e.g., the stomach, upon oral administration. In particular embodiments, a tablet with an aqueous-based film coating comprises Compound 1 as the API. In one embodiment, a tablet with an aqueous-based film coating comprises Compound 1A as the API.

In certain embodiments, provided herein is a controlled release pharmaceutical formulation for oral administration of a co-crystal provided herein, wherein the release occurs substantially in the stomach, comprising: a) a specific amount of a co-crystal provided herein; b) a drug release controlling component for controlling the release of a co-crystal provided herein substantially in the upper gastrointestinal tract, e.g., the stomach; and c) optionally one or more excipients. In certain embodiments, the oral dosage form comprising a co-crystal provided herein is prepared as a controlled release tablet or capsule which includes a drug core comprising the pharmaceutical composition and optional excipients. Optionally, a “seal coat” or “shell” is applied. In certain embodiments, a formulation provided herein comprising a co-crystal provided herein is a controlled release tablet or capsule, which comprises a therapeutically effective amount of a co-crystal provided herein, a drug release controlling component that controls the release of Compound 1 substantially in the stomach upon oral administration, and optionally, one or more excipients.

Particular embodiments provide a drug release controlling component that is a polymer matrix, which swells upon exposure to gastric fluid to effect the gastric retention of the formulation and the sustained release of Compound 1 from the polymer matrix substantially in the stomach. In certain embodiments, such formulations may be prepared by incorporating a co-crystal provided herein into a suitable polymeric matrix during formulation. Examples of such formulations are known in the art. See, e.g., Shell et al., U.S. Patent Publication No. 2002/0051820 (application Ser. No. 09/990,061); Shell et al., U.S. Patent Publication No. 2003/0039688 (application Ser. No. 10/045,823); Gusler et al., U.S. Patent Publication No. 2003/0104053 (application Ser. No. 10/029,134), each of which is incorporated herein by reference in its entirety.

In certain embodiments, the drug release controlling component may comprise a shell surrounding the drug-containing core, wherein the shell releases Compound 1 from the core by, e.g., permitting diffusion of Compound 1 from the core and promoting gastric retention of the formulation by swelling upon exposure to gastric fluids to a size that is retained in the stomach. In certain embodiments, such formulations may be prepared by first compressing a mixture of a co-crystal provided herein and one or more excipients to form a drug core, and compressing another powdered mixture over the drug core to form the shell, or enclosing the drug core with a capsule shell made of suitable materials. Examples of such formulations are known in the art. See, e.g., Berner et al., U.S. Patent Publication No. 2003/0104062 application Ser. No. 10/213,823), incorporated herein by reference in its entirety.

In certain embodiments, the pharmaceutical formulations provided herein contain a co-crystal provided herein and, optionally, one or more excipients to form a “drug core.” Optional excipients include, e.g., diluents (bulking agents), lubricants, disintegrants, fillers, stabilizers, surfactants, preservatives, coloring agents, flavoring agents, binding agents, excipient supports, glidants, permeation enhancement excipients, plasticizers and the like, e.g., as known in the art. It will be understood by those in the art that some substances serve more than one purpose in a pharmaceutical composition. For instance, some substances are binders that help hold a tablet together after compression, yet are also disintegrants that help break the tablet apart once it reaches the target delivery site. Selection of excipients and amounts to use may be readily determined by the formulation scientist based upon experience and consideration of standard procedures and reference works available in the art.

In certain embodiments, formulations provided herein comprise one or more binders. Binders may be used, e.g., to impart cohesive qualities to a tablet, and thus ensure that the tablet remains intact after compression. Suitable binders include, but are not limited to, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropylmethylcellulose, methyl cellulose, ethyl cellulose, hydroxyethyl cellulose, carboxymethyl cellulose and the like), veegum, carbomer (e.g., carbopol), sodium, dextrin, guar gum, hydrogenated vegetable oil, magnesium aluminum silicate, maltodextrin, polymethacrylates, povidone (e.g., KOLLIDON, PLASDONE), microcrystalline cellulose, among others. Binding agents also include, e.g., acacia, agar, alginic acid, cabomers, carrageenan, cellulose acetate phthalate, ceratonia, chitosan, confectioner's sugar, copovidone, dextrates, dextrin, dextrose, ethylcellulose, gelatin, glyceryl behenate, guar gum, hydroxyethyl cellulose, hydroxyethylmethyl cellulose, hydroxypropyl cellulose, hydroxypropyl starch, hypromellose, inulin, lactose, magnesium aluminum silicate, maltodextrin, maltose, methylcellulose, poloxamer, polycarbophil, polydextrose, polyethylene oxide, polymethylacrylates, povidone, sodium alginate, sodium carboxymethylcellulose, starch, pregelatinized starch, stearic acid, sucrose, and zein. The binding agent can be, relative to the drug core, in the amount of about 2% w/w of the drug core; about 4% w/w of the drug core, about 6% w/w of the drug core, about 8% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, about 34% w/w of the drug core, about 36% w/w of the drug core, about 38% w/w of the drug core, about 40% w/w of the drug core, about 42% w/w of the drug core, about 44% w/w of the drug core, about 46% w/w of the drug core, about 48% w/w of the drug core, about 50% w/w of the drug core, about 52% w/w of the drug core, about 54% w/w of the drug core, about 56% w/w of the drug core, about 58% w/w of the drug core, about 60% w/w of the drug core, about 62% w/w of the drug core, about 64% w/w of the drug core, about 66% w/w of the drug core; about 68% w/w of the drug core, about 70% w/w of the drug core, about 72% w/w of the drug core, about 74% w/w of the drug core, about 76% w/w of the drug core, about 78% w/w of the drug core, about 80% w/w of the drug core, about 82% w/w of the drug core, about 84% w/w of the drug core, about 86% w/w of the drug core, about 88% w/w of the drug core, about 90% w/w of the drug core, about 92% w/w of the drug core, about 94% w/w of the drug core, about 96% w/w of the drug core, about 98% w/w of the drug core, or more, if determined to be appropriate. In certain embodiments, a suitable amount of a particular binder is determined by one of ordinary skill in the art.

In certain embodiments, formulations provided herein comprise one or more diluents. Diluents may be used, e.g., to increase bulk so that a practical size tablet is ultimately provided. Suitable diluents include dicalcium phosphate, calcium sulfate, lactose, cellulose, kaolin, mannitol, sodium chloride, dry starch, microcrystalline cellulose (e.g., AVICEL), microfine cellulose, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT), potassium chloride, sodium chloride, sorbitol and talc, among others. Diluents also include, e.g., ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-β-cyclodextrin, talc, tragacanth, trehalose, and xylitol. Diluents may be used in amounts calculated to obtain a desired volume for a tablet or capsule; in certain embodiments, a diluent is used in an amount of about 5% or more, about 10% or more, about 15% or more, about 20% or more, about 22% or more, about 24% or more, about 26% or more, about 28% or more, about 30% or more, about 32% or more, about 34% or more, about 36% or more, about 38% or more, about 40% or more, about 42% or more, about 44% or more, about 46% or more, about 48% or more, about 50% or more, about 52% or more, about 54% or more, about 56% or more, about 58% or more, about 60% or more, about 62% or more, about 64% or more, about 68% or more, about 70% ore more, about 72% or more, about 74% or more, about 76% or more, about 78% or more, about 80% or more, about 85% or more, about 90% or more, or about 95% or more, weight/weight, of a drug core; between about 10% and about 90% w/w of the drug core; between about 20% and about 80% w/w of the drug core; between about 30% and about 70% w/w of the drug core; between about 40% and about 60% w/w of the drug core. In certain embodiments, a suitable amount of a particular diluent is determined by one of ordinary skill in the art.

In certain embodiments, formulations provided herein comprise one or more lubricants. Lubricants may be used, e.g., to facilitate tablet manufacture; examples of suitable lubricants include, for example, vegetable oils such as peanut oil, cottonseed oil, sesame oil, olive oil, corn oil, and oil of theobroma, glycerin, magnesium stearate, calcium stearate, and stearic acid. In certain embodiments, stearates, if present, represent no more than approximately 2 weight % of the drug-containing core. Further examples of lubricants include, e.g., calcium stearate, glycerin monostearate, glyceryl behenate, glyceryl palmitostearate, magnesium lauryl sulfate, magnesium stearate, myristic acid, palmitic acid, poloxamer, polyethylene glycol, potassium benzoate, sodium benzoate, sodium chloride, sodium lauryl sulfate, sodium stearyl fumarate, stearic acid, talc, and zinc stearate. In particular embodiments, the lubricant is magnesium stearate. In certain embodiments, the lubricant is present, relative to the drug core, in an amount of about 0.2% w/w of the drug core, about 0.4% w/w of the drug core, about 0.6% w/w of the drug core, about 0.8% w/w of the drug core, about 1.0% w/w of the drug core, about 1.2% w/w of the drug core, about 1.4% w/w of the drug core, about 1.6% w/w of the drug core, about 1.8% w/w of the drug core, about 2.0% w/w of the drug core, about 2.2% w/w of the drug core, about 2.4% w/w of the drug core, about 2.6% w/w of the drug core, about 2.8% w/w of the drug core, about 3.0% w/w of the drug core, about 3.5% w/w of the drug core, about 4% w/w of the drug core, about 4.5% w/w of the drug core, about 5% w/w of the drug core, about 6% w/w of the drug core, about 7% w/w of the drug core, about 8% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 25% w/w of the drug core, about 30% w/w of the drug core, about 35% w/w of the drug core, about 40% w/w of the drug core, between about 0.2% and about 10% w/w of the drug core, between about 0.5% and about 5% w/w of the drug core, or between about 1% and about 3% w/w of the drug core. In certain embodiments, a suitable amount of a particular lubricant is determined by one of ordinary skill in the art.

In certain embodiments, formulations provided herein comprise one or more disintegrants. Disintegrants may be used, e.g., to facilitate disintegration of the tablet, and may be, e.g., starches, clays, celluloses, algins, gums or crosslinked polymers. Disintegrants also include, e.g., alginic acid, carboxymethylcellulose calcium, carboxymethylcellulose sodium (e.g., AC-DI-SOL, PRIMELLOSE), colloidal silicon dioxide, croscarmellose sodium, crospovidone (e.g., KOLLIDON, POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB) and starch. Additional disintegrants include, e.g., calcium alginate, chitosan, sodium docusate, hydroxypropyl cellulose, and povidone. In certain embodiments, the disintegrant is, relative to the drug core, present in the amount of about 1% w/w of the drug core, about 2% w/w of the drug core, about 3% w/w of the drug core, about 4% w/w of the drug core, about 5% w/w of the drug core, about 6% w/w of the drug core, about 7% w/w of the drug core, about 8% w/w of the drug core, about 9% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, greater than about 32% w/w of the drug core, between about 1% and about 10% w/w of the drug core, between about 2% and about 8% w/w of the drug core, between about 3% and about 7% w/w of the drug core, or between about 4% and about 6% w/w of the drug core. In certain embodiments, a suitable amount of a particular disintegrant is determined by one of ordinary skill in the art.

In certain embodiments, formulations provided herein comprise one or more stabilizers. Stabilizers (also called absorption enhancers) may be used, e.g., to inhibit or retard drug decomposition reactions that include, by way of example, oxidative reactions. Stabilizing agents include, e.g., d-alpha-tocopheryl polyethylene glycol 1000 succinate (Vitamin E TPGS), acacia, albumin, alginic acid, aluminum stearate, ammonium alginate, ascorbic acid, ascorbyl palmitate, bentonite, butylated hydroxytoluene, calcium alginate, calcium stearate, calcium carboxymethylcellulose, carrageenan, ceratonia, colloidal silicon dioxide, cyclodextrins, diethanolamine, edetates, ethylcellulose, ethyleneglycol palmitostearate, glycerin monostearate, guar gum, hydroxypropyl cellulose, hypromellose, invert sugar, lecithin, magnesium aluminum silicate, monoethanolamine, pectin, poloxamer, polyvinyl alcohol, potassium alginate, potassium polacrilin, povidone, propyl gallate, propylene glycol, propylene glycol alginate, raffinose, sodium acetate, sodium alginate, sodium borate, sodium carboxymethyl cellulose, sodium stearyl fumarate, sorbitol, stearyl alcohol, sulfobutyl-b-cyclodextrin, trehalose, white wax, xanthan gum, xylitol, yellow wax, and zinc acetate. In certain embodiments, the stabilizer is, relative to the drug core, present in the amount of about 1% w/w of the drug core, about 2% w/w of the drug core, about 3% w/w of the drug core, about 4% w/w of the drug core, about 5% w/w of the drug core, about 6% w/w of the drug core, about 7% w/w of the drug core, about 8% w/w of the drug core, about 9% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, between about 1% and about 10% w/w of the drug core, between about 2% and about 8% w/w of the drug core, between about 3% and about 7% w/w of the drug core, or between about 4% and about 6% w/w of the drug core. In certain embodiments, a suitable amount of a particular stabilizer is determined by one of ordinary skill in the art.

In certain embodiments, formulations provided herein comprise one or more glidants. Glidants may be used, e.g., to improve the flow properties of a powder composition or granulate or to improve the accuracy of dosing. Excipients that may function as glidants include, e.g., colloidal silicon dioxide, magnesium trisilicate, powdered cellulose, starch, tribasic calcium phosphate, calcium silicate, powdered cellulose, colloidal silicon dioxide, magnesium silicate, magnesium trisilicate, silicon dioxide, starch, tribasic calcium phosphate, and talc. In certain embodiments, the glidant is, relative to the drug core, present in the amount of less than about 1% w/w of the drug core, about 1% w/w of the drug core, about 2% w/w of the drug core, about 3% w/w of the drug core, about 4% w/w of the drug core, about 5% w/w of the drug core, about 6% w/w of the drug core, about 7% w/w of the drug core, about 8% w/w of the drug core, about 9% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, between about 1% and about 10% w/w of the drug core, between about 2% and about 8% w/w of the drug core, between about 3% and about 7% w/w of the drug core, or between about 4% and about 6% w/w of the drug core. In certain embodiments, a suitable amount of a particular glidant is determined by one of ordinary skill in the art.

In certain embodiments, the pharmaceutical compositions provided herein may be administered orally, parenterally, by inhalation spray, topically, rectally, nasally, buccally, vaginally or via an implanted reservoir, preferably by oral administration or administration by injection. In one embodiment, the pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

In certain embodiments, the pharmaceutical compositions provided herein may be in the form of a sterile injectable preparation, for example, as a sterile injectable aqueous or oleaginous suspension. This suspension may be formulated according to techniques known in the art using suitable dispersing or wetting agents (such as, for example, TWEEN 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs or SPANs and/or other similar emulsifying agents or bioavailability enhancers which are commonly used in the manufacture of pharmaceutically acceptable solid, liquid, or other dosage forms may also be used for the purposes of formulation.

In certain embodiments, the pharmaceutical compositions provided herein may also be administered in the form of suppositories for rectal administration. These compositions can be prepared by mixing a co-crystal provided herein with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions provided herein is useful when the desired treatment involves areas or organs readily accessible by topical application. For application topically to the skin, the pharmaceutical composition should be formulated with a suitable ointment containing the active components suspended or dissolved in a carrier. In certain embodiments, carriers for topical administration of the compounds provided herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions provided herein may also be topically applied to the lower intestinal tract by rectal suppository formulation or in a suitable enema formulation. Topically-transdermal patches are also included herein.

In certain embodiments, the pharmaceutical compositions provided herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In certain embodiments, the compositions provided herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. In one embodiment, the pharmaceutical compositions are administered from about 1 to about 6 times per day or alternatively, as a continuous infusion. Such administration can be used as a chronic or acute therapy. The amount of active ingredient that may be combined with the carrier materials to produce a single dosage form varies depending upon the host treated and the particular mode of administration. A typical preparation contains from about 5% to about 95% active compound (w/w). Alternatively, such preparations contain from about 20% to about 80% active compound.

Methods of Use

The co-crystals provided herein are useful for treating a disease selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma, lessen the severity of the disease/disorder (AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, or cholangiocarcinoma, each characterized by the presence of a mutant allele of IDH2). In one embodiment, the co-crystal for use in the methods is a co-crystal comprising Compound 1A.

In one embodiment, provided herein is a method of treating and preventing a disease or condition, comprising the administration of a co-crystal comprising Compound 1, wherein the disease is selected from AML, MDS, CMML, myeloid sarcoma, multiple myeloma, lymphoma (e.g., T-cell lymphoma and B-cell lymphoma), AITL, blastic plasmacytoid dendritic cell neoplasm, MPN, glioma, melanoma, chondrosarcoma, and cholangiocarcinoma, lessen the severity of the disease/disorder (AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, or cholangiocarcinoma, each characterized by the presence of a mutant allele of IDH2).

In one embodiment, provided herein is a method of treating AML selected from newly diagnosed AML, previously untreated AML, AML arising from MDS, AML arising from antecedent hematological disorder (AHD) and AML arising after exposure to genotoxic injury. In certain embodiments, the genotoxic injury is resulting from radiation and/or chemotherapy. In one embodiment, provided herein is a method of treating AML arising after exposure to genotoxic injury resulting from radiation and/or chemotherapy), each characterized by the presence of a mutant allele of IDH2.

In one embodiment, provided herein is a method of treating newly diagnosed AML characterized by the presence of a mutant allele of IDH2.

In one embodiment, provided herein is a method of treating previously untreated AML characterized by the presence of a mutant allele of IDH2.

In one embodiment, provided herein is a method of treating AML arising from MDS characterized by the presence of a mutant allele of IDH2.

In one embodiment, provided herein is a method of treating AML arising from AHD characterized by the presence of a mutant allele of IDH2.

In one embodiment, provided herein is a method of treating AML arising after exposure to genotoxic injury characterized by the presence of a mutant allele of IDH2.

In one embodiment, provided herein is a method of treating myeloproliferative neoplasm (MPN).

In one aspect of this embodiment, the mutant IDH2 has an R140X mutation. In another aspect of this embodiment, the R140X mutation is a R140Q mutation. In another aspect of this embodiment, the R140X mutation is a R140W mutation. In another aspect of this embodiment, the R140X mutation is a R140L mutation. In another aspect of this embodiment, the mutant IDH2 has an R172X mutation. In another aspect of this embodiment, the R172X mutation is a R172K mutation. In another aspect of this embodiment, the R172X mutation is a R172G mutation. A cancer selected from AML, MDS, CMML, or lymphoma (e.g., T-cell lymphoma) can be analyzed by sequencing cell samples to determine the presence and specific nature of (e.g., the changed amino acid present at) a mutation at amino acid 140 and/or 172 of IDH2.

Without being bound by theory, applicants believe that mutant alleles of IDH2 wherein the IDH2 mutation results in a new ability of the enzyme to catalyze the NADPH-dependent reduction of α-ketoglutarate to R(−)-2-hydroxyglutarate, and in particular R140Q and/or R172K mutations of IDH2, characterize a subset of all types of cancers described herein, without regard to their cellular nature or location in the body. Thus, the methods of one aspect are useful to treat a hematological cancer selected from AML, MDS, CMML, or lymphoma (e.g., T-cell lymphoma) or solid tumor selected from glioma, melanoma, chondrosarcoma, cholangiocarcinoma (e.g., glioma) and AITL, that is characterized by the presence of a mutant allele of IDH2 imparting such activity and in particular an IDH2 R140Q and/or R172K mutation.

In one embodiment, the efficacy of treatment is monitored by measuring the levels of 2HG in the subject. Typically levels of 2HG are measured prior to treatment, wherein an elevated level is indicated for the use of Compound 1 to treat a disease selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma. Once the elevated levels are established, the level of 2HG is determined during the course of and/or following termination of treatment to establish efficacy. In certain embodiments, the level of 2HG is only determined during the course of and/or following termination of treatment. A reduction of 2HG levels during the course of treatment and following treatment is indicative of efficacy. Similarly, a determination that 2HG levels are not elevated during the course of or following treatment is also indicative of efficacy. Typically, the these 2HG measurements will be utilized together with other well-known determinations of efficacy of cancer treatment, such as reduction in number and size of tumors and/or other cancer-associated lesions, improvement in the general health of the subject, and alterations in other biomarkers that are associated with cancer treatment efficacy.

2HG can be detected in a sample by the methods of PCT Publication No. WO 2013/102431 and US Publication No. US 2013/0190287 hereby incorporated by reference in their entirety, or by analogous methods.

In one embodiment 2HG is directly evaluated.

In another embodiment a derivative of 2HG formed in process of performing the analytic method is evaluated. By way of example such a derivative can be a derivative formed in MS analysis. Derivatives can include a salt adduct, e.g., a Na adduct, a hydration variant, or a hydration variant which is also a salt adduct, e.g., a Na adduct, e.g., as formed in MS analysis.

In another embodiment a metabolic derivative of 2HG is evaluated. Examples include species that build up or are elevated, or reduced, as a result of the presence of 2HG, such as glutarate or glutamate that will be correlated to 2HG, e.g., R-2HG.

Exemplary 2HG derivatives include dehydrated derivatives such as the compounds provided below or a salt adduct thereof:

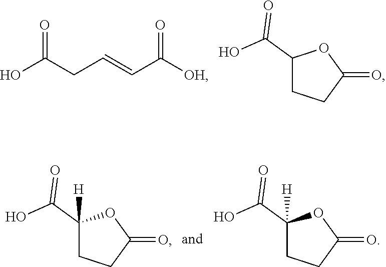 [SMILES: O=C(O)/C=C/CC(=O)O ; O=C1CCC(C(=O)O)O1 ; O=C1CC[C@H](C(=O)O)O1 ; O=C1CC[C@@H](C(=O)O)O1]

In one embodiment the disease selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma, wherein at least 30, 40, 50, 60, 70, 80 or 90% of the tumor cells carry an IDH2 mutation, and in particular an IDH2 R140Q, R140W, or R140L and/or R172K or R172G mutation, at the time of diagnosis or treatment.

In one embodiment, the cancer to be treated is AML. In some embodiments, the AML is relapsed and/or primary refractory. In some embodiments, the AML is relapsed and/or refractory. In other embodiments, the AML is previously untreated. In one embodiment, the AML is newly diagnosed AML.

In another embodiment, the cancer to be treated is MDS with refractory anemia with excess blasts (subtype RAEB-1 or RAEB-2). In other embodiments, the MDS is previously untreated. In one embodiment, the MDS is newly diagnosed MDS.

In another embodiment, the cancer to be treated is relapsed and/or primary refractory CMML.

In certain embodiments, the co-crystals provided herein are for treating a hematological malignancy characterized by the presence of a mutant allele of IDH2 and the absence of a mutant allele of FLT3 and/or a mutant allele of NRAS. Exemplary methods for treating a hematological malignancy characterized by the presence of a mutant allele of IDH2 and the absence of a mutant allele of FLT3 and/or a mutant allele of NRAS by administering Compound 1 are described in US 2017/024617 and US 2017/0157132, the disclosure of each of which is incorporated herein by reference in its entirety.

In one embodiment, the co-crystals provided herein are for treating a hematological malignancy characterized by the presence of a mutant allele of IDH2 and the absence of a mutant allele of FLT3. In one embodiment, the hematological malignancy is an advanced hematological malignancy. In one embodiment, the hematological malignancy is AML. In some embodiments, the AML is relapsed and/or refractory.

In one embodiment, provided herein are methods of treating a hematological malignancy by administering a co-crystal comprising Compound 1 in combination with a therapeutically effective amount of one or more compounds that target a FLT3 pathway, wherein the hematological malignancy is characterized by the presence of a mutant allele of IDH2 and a mutant allele of FLT3, for example FLT3-ITD or FLT3-KDM. In one embodiment, the hematological malignancy is an advanced hematological malignancy. In one embodiment, the hematological malignancy is AML. In some embodiments, the AML is relapsed and/or refractory.

In one embodiment, provided herein is a method of treating hematological malignancies, such as AML, MDS, CMML, myeloid sarcoma, multiple myeloma, lymphoma (e.g., T-cell lymphoma or B-cell lymphoma), AITL or blastic plasmacytoid dendritic cell neoplasm, each characterized by the presence of a mutant allele of IDH2 and the absence of a mutant allele of FLT3, comprising administering a co-crystal comprising Compound 1. In one embodiment, the hematological malignancy is an advanced hematological malignancy. In one embodiment, the hematological malignancy is AML. In some embodiments, the AML is relapsed and/or refractory.

In one embodiment, provided herein is a method of treating hematological malignancies, such as AML, MDS, CMML, myeloid sarcoma, multiple myeloma, lymphoma (e.g., T-cell lymphoma or B-cell lymphoma), AITL or blastic plasmacytoid dendritic cell neoplasm, each characterized by the presence of a mutant allele of IDH2 and a mutant allele of FLT3, for example FLT3-ITD, comprising administering a co-crystal comprising Compound 1 in combination with a therapeutically effective amount of one or more compounds that target a FLT3 pathway. Exemplary FLT3 inhibitors are described elsewhere herein. In one embodiment, the hematological malignancy is an advanced hematological malignancy. In one embodiment, the hematological malignancy is AML. In some embodiments, the AML is relapsed and/or refractory.

In one embodiment, provided herein are methods of treating solid tumors by administering a co-crystal comprising Compound 1, wherein the solid tumor is characterized by the presence of a mutant allele of IDH2 and the absence of a mutant allele of FLT3. In one embodiment, the solid tumor is an advanced solid tumor. In some embodiments, the AML is relapsed and/or refractory.

In one embodiment, provided herein are methods of treating solid tumors by administering to a subject a co-crystal comprising Compound 1 in combination with a therapeutically effective amount of one or more compounds that target a FLT3 pathway, wherein the solid tumor is characterized by the presence of a mutant IDH2 and a mutant allele of FLT3, for example FLT3-ITD. In one embodiment, the solid tumor is an advanced solid tumor.

In one embodiment, provided herein is a method of treating solid tumors, such as glioma, melanoma, chondrosarcoma, or cholangiocarcinoma(e.g., glioma), or treating AITL, each characterized by the presence of a mutant allele of IDH2 and the absence of a mutant allele of FLT3, comprising administering to a subject a co-crystal provided herein.

In one embodiment, provided herein is a method of treating solid tumors, such as glioma, melanoma, chondrosarcoma, or cholangiocarcinoma (e.g., glioma), or treating AITL, each characterized by the presence of a mutant allele of IDH2 and a mutant allele of FLT3, in a subject comprising administering a co-crystal comprising Compound 1 in combination with a therapeutically effective amount of one or more compounds that target a FLT3 pathway. Exemplary FLT3 inhibitors are described elsewhere herein.

In one embodiment, provided herein is a method of treating a hematological malignancy by administering a co-crystal comprising Compound 1, wherein the hematological malignancy is characterized by the presence of a mutant allele of IDH2 and the absence of a mutant allele of NRAS. In one embodiment, the hematological malignancy is an advanced hematological malignancy.

In one embodiment, provided herein is a method of treating a hematological malignancy by administering a co-crystal comprising Compound 1 in combination with a therapeutically effective amount of one or more compounds that target RAS pathways, wherein the hematological malignancy is characterized by the presence of a mutant allele of IDH2 and a mutant allele of NRAS. In one embodiment, the hematological malignancy is an advanced hematological malignancy.

In one embodiment, provided herein is a method of treating a hematological malignancy, such as AML, MDS, CMML, myeloid sarcoma, multiple myeloma, lymphoma (e.g., T-cell lymphoma or B-cell lymphoma), AITL or blastic plasmacytoid dendritic cell neoplasm, each characterized by the presence of a mutant allele of IDH2 and the absence of a mutant allele of NRAS, comprising administering a co-crystal comprising Compound 1. In one embodiment, the hematological malignancy is an advanced hematological malignancy.

In one embodiment, provided herein is a method of treating hematological malignancies, such as AML, MDS, CMML, myeloid sarcoma, multiple myeloma, lymphoma (e.g., T-cell lymphoma or B-cell lymphoma), AITL or blastic plasmacytoid dendritic cell neoplasm, each characterized by the presence of a mutant allele of IDH2 and a mutant allele of NRAS comprising administering a co-crystal comprising Compound 1 in combination with a therapeutically effective amount of one or more compounds that target RAS pathways. In one embodiment, a co-crystal comprising Compound 1 is administered to the subject in combination with a therapeutically effective amount of a MEK kinase inhibitor. Exemplary MEK kinase inhibitors are described elsewhere herein. In one embodiment, the hematological malignancy is an advanced hematological malignancy.

In one embodiment, provided herein are methods of treating solid tumors by administering a co-crystal comprising Compound 1, wherein the solid tumor is characterized by the presence of a mutant allele of IDH2 and the absence of a mutant allele of NRAS. In one embodiment, the solid tumor is an advanced solid tumor.

In one embodiment, provided herein are methods of treating solid tumors by administering a co-crystal comprising Compound 1 in combination with a therapeutically effective amount of one or more compounds that target RAS pathways, wherein the solid tumor is characterized by the presence of a mutant IDH2 and a mutant allele of NRAS. In one embodiment, the solid tumor is an advanced solid tumor.

In one embodiment, provided herein is a method of treating solid tumors, such as glioma, melanoma, chondrosarcoma, or cholangiocarcinoma(e.g., glioma), or treating angioimmunoblastic T-cell lymphoma (AITL), each characterized by the presence of a mutant allele of IDH2 and the absence of a mutant allele of NRAS, comprising administering a co-crystal comprising Compound 1.

In one embodiment, provided herein is a method of treating solid tumors, such as glioma, melanoma, chondrosarcoma, or cholangiocarcinoma (e.g., glioma), or treating angioimmunoblastic T-cell lymphoma (AITL), each characterized by the presence of a mutant allele of IDH2 and a mutant allele of NRAS, comprising administering a co-crystal comprising Compound 1 in combination with a therapeutically effective amount of one or more compounds that target RAS pathways.

In one embodiment, provided herein are methods of treating MPN in a subject comprising administering to the subject a co-crystal comprising Compound 1 in combination with a therapeutically effective amount of a JAK2 inhibitor, wherein the subject harbors a mutant allele of IDH2 and a mutant allele of JAK2. Exemplary JAK2 inhibitors are described elsewhere herein.

In certain embodiments, provided herein is a method of treating a high risk MPN in a subject comprising administering to the subject a co-crystal comprising Compound 1 in combination with a therapeutically effective amount of a JAK2 inhibitor, wherein the subject harbors a mutant allele of IDH2 and a mutant allele of JAK2.

In one embodiment, provided herein are methods of treating AML in a subject comprising administering to the subject a co-crystal comprising Compound 1 in combination with a therapeutically effective amount of a JAK2 inhibitor, wherein the subject harbors a mutant allele of IDH2 and a mutant allele of JAK2. In some embodiments, the AML is relapsed and/or refractory.

In certain embodiments, the mutant allele of IDH2 is mIDH2-R140 or mIDH2-R172.

In certain embodiments, the mutant allele of IDH2 is mIDH2-R140Q, mIDH2-R140W, mIDH2-R140L, mIDH2-R172K, or mIDH2-R172G.

In certain embodiments, the mutant allele of JAK2 is mJAK2-V617F.

In certain embodiments, the co-crystals provided herein are for treating MDS characterized by the presence of a mutant allele of IDH2 and a mutant allele of at least one second gene, wherein the second gene is selected from the group consisting of ASXL1 and SRSF2. In certain embodiments, the co-crystals provided herein are for treating MDS characterized by the presence of a mutant allele of IDH2 and the absence of a mutant allele of at least one other gene, wherein the other gene is selected from the group consisting of KRAS, TP53, SETBP1, and U2AF1. In certain embodiments, the co-crystals provided herein are for treating MDS characterized by the presence of a mutant allele of IDH2 and the absence of a mutant allele of at least one other gene, wherein the other gene is selected from the group consisting of KRAS, TP53, SETBP1, U2AF1, TCF3, STAG2, NRAS, JAK2 and BRAF. Exemplary methods of treating MDS characterized by the presence of a mutant allele of IDH2 by administering Compound 1 are described in US 2018/0042930-A1, the disclosure of which is incorporated herein by reference in its entirety.

In one embodiment, prior to and/or after treatment with a co-crystal comprising Compound 1 provided herein, the method further comprises the step of evaluating the growth, size, weight, invasiveness, stage and/or other phenotype of the cancer selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma.

In one embodiment, prior to and/or after treatment with a composition provided herein, the method further comprises the step of evaluating the IDH2 genotype of the cancer selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma. This may be achieved by ordinary methods in the art, such as DNA sequencing, immuno analysis, and/or evaluation of the presence, distribution or level of 2HG.

In one embodiment, prior to and/or after treatment with a composition provided herein, the method further comprises the step of determining the 2HG level in the subject. This may be achieved by spectroscopic analysis, e.g., magnetic resonance-based analysis, e.g., MRI and/or MRS measurement, sample analysis of bodily fluid, such as blood, plasma, urine, or spinal cord fluid analysis, or by analysis of surgical material, e.g., by mass-spectroscopy (e.g. LC-MS, GC-MS).

In one embodiment, the co-crystal comprising Compound 1 is for use in any of the above described methods. In one embodiment, the co-crystal for use in the methods is a co-crystal comprising Compound 1A. In one embodiment, the co-crystal for use in the methods is a mixture co-crystal comprising Compound 1A.

In certain embodiments, depending on the disease to be treated and the subject's condition, the co-crystal provided herein may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The co-crystal provided herein may be formulated alone or together with one or more active agent(s), in suitable dosage unit with pharmaceutically acceptable excipients, carriers, adjuvants and vehicles, appropriate for each route of administration.

In certain embodiments, the amount of the co-crystal provided herein administered in the methods provided herein may range, e.g., between about 5 mg/day and about 2,000 mg/day. In one embodiment, the range is between about 10 mg/day and about 2,000 mg/day. In one embodiment, the range is between about 20 mg/day and about 2,000 mg/day. In one embodiment, the range is between about 50 mg/day and about 1,000 mg/day. In one embodiment, the range is between about 100 mg/day and about 1,000 mg/day. In one embodiment, the range is between about 100 mg/day and about 500 mg/day. In one embodiment, the range is between about 150 mg/day and about 500 mg/day. In one embodiment, the range is or between about 150 mg/day and about 250 mg/day. In certain embodiments, particular dosages are, e.g., about 10 mg/day. In one embodiment, the dose is about 20 mg/day. In one embodiment, the dose is about 50 mg/day. In one embodiment, the dose is about 60 mg/day. In one embodiment, the dose is about 75 mg/day. In one embodiment, the dose is about 100 mg/day. In one embodiment, the dose is about 120 mg/day. In one embodiment, the dose is about 150 mg/day. In one embodiment, the dose is about 200 mg/day. In one embodiment, the dose is about 250 mg/day. In one embodiment, the dose is about 300 mg/day. In one embodiment, the dose is about 350 mg/day. In one embodiment, the dose is about 400 mg/day. In one embodiment, the dose is about 450 mg/day. In one embodiment, the dose is about 500 mg/day. In one embodiment, the dose is about 600 mg/day. In one embodiment, the dose is about 700 mg/day. In one embodiment, the dose is about 800 mg/day. In one embodiment, the dose is about 900 mg/day. In one embodiment, the dose is about 1,000 mg/day. In one embodiment, the dose is about 1,200 mg/day. In one embodiment, the dose is or about 1,500 mg/day. In certain embodiments, particular dosages are, e.g., up to about 10 mg/day. In one embodiment, the particular dose is up to about 20 mg/day. In one embodiment, the particular dose is up to about 50 mg/day. In one embodiment, the particular dose is up to about 60 mg/day. In one embodiment, the particular dose is up to about 75 mg/day. In one embodiment, the particular dose is up to about 100 mg/day. In one embodiment, the particular dose is up to about 120 mg/day. In one embodiment, the particular dose is up to about 150 mg/day. In one embodiment, the particular dose is up to about 200 mg/day. In one embodiment, the particular dose is up to about 250 mg/day. In one embodiment, the particular dose is up to about 300 mg/day. In one embodiment, the particular dose is up to about 350 mg/day. In one embodiment, the particular dose is up to about 400 mg/day. In one embodiment, the particular dose is up to about 450 mg/day. In one embodiment, the particular dose is up to about 500 mg/day. In one embodiment, the particular dose is up to about 600 mg/day. In one embodiment, the particular dose is up to about 700 mg/day. In one embodiment, the particular dose is up to about 800 mg/day. In one embodiment, the particular dose is up to about 900 mg/day. In one embodiment, the particular dose is up to about 1,000 mg/day. In one embodiment, the particular dose is up to about 1,200 mg/day. In one embodiment, the particular dose is up to about 1,500 mg/day.

In certain embodiments, the co-crystal provided herein for methods described herein is administered at a dose of about 20 to 2000 mg/day. In certain embodiments, the co-crystal provided herein is administered at a dose of about 50 to 500 mg/day. In certain embodiments, the dose is about 60 mg/day. In certain embodiments, the dose is about 100 mg/day. In certain embodiments, the dose is about 150 mg/day. In certain embodiments, the dose is about 200 mg/day. In certain embodiments, the dose is about 300 mg/day.

In one embodiment, the amount of the co-crystal provided herein in the pharmaceutical composition or dosage form provided herein may range, e.g., between about 5 mg and about 2,000 mg. In one embodiment, the range is between about 10 mg and about 2,000 mg. In one embodiment, the range is between about 20 mg and about 2,000 mg. In one embodiment, the range is between about 50 mg and about 1,000 mg. In one embodiment, the range is between about 50 mg and about 500 mg. In one embodiment, the range is between about 50 mg and about 250 mg. In one embodiment, the range is between about 100 mg and about 500 mg. In one embodiment, the range is between about 150 mg and about 500 mg. In one embodiment, the range is between about 150 mg and about 250 mg. In certain embodiments, particular amounts are, e.g., about 10 mg. In one embodiment, the particular amount is about 20 mg. In one embodiment, the particular amount is about 30 mg. In one embodiment, the particular amount is about 50 mg. In one embodiment, the particular amount is about 60 mg. In one embodiment, the particular amount is about 75 mg. In one embodiment, the particular amount is about 100 mg. In one embodiment, the particular amount is about 120 mg. In one embodiment, the particular amount is about 150 mg. In one embodiment, the particular amount is about 200 mg. In one embodiment, the particular amount is about 250 mg. In one embodiment, the particular amount is about 300 mg. In one embodiment, the particular amount is about 350 mg. In one embodiment, the particular amount is about 400 mg. In one embodiment, the particular amount is about 450 mg. In one embodiment, the particular amount is about 500 mg. In one embodiment, the particular amount is about 600 mg. In one embodiment, the particular amount is about 650 mg. In one embodiment, the particular amount is about 700 mg. In one embodiment, the particular amount is about 800 mg. In one embodiment, the particular amount is about 900 mg. In one embodiment, the particular amount is about 1,000 mg. In one embodiment, the particular amount is about 1,200 mg. In one embodiment, the particular amount is or about 1,500 mg. In certain embodiments, particular amounts are, e.g., up to about 10 mg. In one embodiment, the particular amount is up to about 20 mg. In one embodiment, the particular amount is up to about 50 mg. In one embodiment, the particular amount is up to about 60 mg. In one embodiment, the particular amount is up to about 75 mg. In one embodiment, the particular amount is up to about 100 mg. In one embodiment, the particular amount is up to about 120 mg. In one embodiment, the particular amount is up to about 150 mg. In one embodiment, the particular amount is up to about 200 mg. In one embodiment, the particular amount is up to about 250 mg. In one embodiment, the particular amount is up to about 300 mg. In one embodiment, the particular amount is up to about 350 mg. In one embodiment, the particular amount is up to about 400 mg. In one embodiment, the particular amount is up to about 450 mg. In one embodiment, the particular amount is up to about 500 mg. In one embodiment, the particular amount is up to about 600 mg. In one embodiment, the particular amount is up to about 700 mg. In one embodiment, the particular amount is up to about 800 mg. In one embodiment, the particular amount is up to about 900 mg. In one embodiment, the particular amount is up to about 1,000 mg. In one embodiment, the particular amount is up to about 1,200 mg. In one embodiment, the particular amount is up to about 1,500 mg.

In one embodiment, the co-crystal provided herein can be delivered as a single dose such as, e.g., a single bolus injection, or oral tablets or pills; or over time such as, e.g., continuous infusion over time or divided bolus doses over time. In one embodiment, the co-crystal provided herein can be administered repetitively if necessary, for example, until the patient experiences stable disease or regression, or until the patient experiences disease progression or unacceptable toxicity. Stable disease or lack thereof is determined by methods known in the art such as evaluation of patient's symptoms, physical examination, visualization of the tumor that has been imaged using X-ray, CAT, PET, or MRI scan and other commonly accepted evaluation modalities.

In certain embodiments, the co-crystal provided herein for methods described herein is administered once daily.

In certain embodiments, the co-crystal provided herein is administered to a patient in cycles (e.g., daily administration for one week, then a rest period with no administration for up to three weeks). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, a method provided herein comprises administering the co-crystal provided herein in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In certain embodiments, the co-crystal provided herein for methods described herein is administered for 1 to 25 cycles. In one embodiment, the median number of cycles administered in a group of patients is about 1. In one embodiment, the median number of cycles administered in a group of patients is about 2. In one embodiment, the median number of cycles administered in a group of patients is about 3. In one embodiment, the median number of cycles administered in a group of patients is about 4. In one embodiment, the median number of cycles administered in a group of patients is about 5. In one embodiment, the median number of cycles administered in a group of patients is about 6. In one embodiment, the median number of cycles administered in a group of patients is about 7. In one embodiment, the median number of cycles administered in a group of patients is about 8. In one embodiment, the median number of cycles administered in a group of patients is about 9. In one embodiment, the median number of cycles administered in a group of patients is about 10. In one embodiment, the median number of cycles administered in a group of patients is about 11. In one embodiment, the median number of cycles administered in a group of patients is about 12. In one embodiment, the median number of cycles administered in a group of patients is about 13. In one embodiment, the median number of cycles administered in a group of patients is about 14. In one embodiment, the median number of cycles administered in a group of patients is about 15. In one embodiment, the median number of cycles administered in a group of patients is about 16. In one embodiment, the median number of cycles administered in a group of patients is about 17. In one embodiment, the median number of cycles administered in a group of patients is about 18. In one embodiment, the median number of cycles administered in a group of patients is about 19. In one embodiment, the median number of cycles administered in a group of patients is about 20. In one embodiment, the median number of cycles administered in a group of patients is about 21. In one embodiment, the median number of cycles administered in a group of patients is about 22. In one embodiment, the median number of cycles administered in a group of patients is about 23. In one embodiment, the median number of cycles administered in a group of patients is about 24. In one embodiment, the median number of cycles administered in a group of patients is about 25. In one embodiment, the median number of cycles administered in a group of patients is about 26. In one embodiment, the median number of cycles administered in a group of patients is about 27. In one embodiment, the median number of cycles administered in a group of patients is about 28. In one embodiment, the median number of cycles administered in a group of patients is about 29. In one embodiment, the median number of cycles administered in a group of patients is about 30. In one embodiment, the median number of cycles administered in a group of patients is greater than about 30 cycles.

In certain embodiments, treatment cycles comprise multiple doses of the co-crystal provided herein administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days).

In certain embodiments, the co-crystal provided herein is administered in one or more 28 day cycles in the methods described herein. In certain embodiments, the co-crystal provided herein is administered in a 28 day cycle in the methods described herein.

In certain embodiments, the co-crystal provided herein is administered orally in the methods described herein.

In certain embodiments, the co-crystal provided herein is administered once daily orally in 28-day cycles at the dose of about 100 mg/day in the methods described herein.

Combination Therapy

In certain embodiments, the co-crystals provided herein are used with an additional cancer therapeutic agent or an additional cancer treatment. Exemplary additional cancer therapeutic agents and additional cancer treatments are described in US 2013/0190287, US 2017/0157132, US 2017/0246174, WO 2017/066611, and WO 2017/066599, and International Application No. PCT/US18/31090, the disclosures of each of which is incorporated herein by reference in their entireties.

In certain embodiments, additional cancer therapeutic agents include for example, chemotherapy, targeted therapy, antibody therapies, immunotherapy, and hormonal therapy. In certain embodiments, additional cancer treatments include, for example: surgery, and radiation therapy. Examples of each of these treatments are provided below.

In some embodiments, the additional cancer therapeutic agent is a chemotherapy agent. Examples of chemotherapeutic agents used in cancer therapy include, for example, antimetabolites (e.g., folic acid, purine, and pyrimidine derivatives), alkylating agents (e.g., nitrogen mustards, nitrosoureas, platinum, alkyl sulfonates, hydrazines, triazenes, aziridines, spindle poison, cytotoxic agents, topoisomerase inhibitors and others), and hypomethylating agents (e.g., decitabine (5-aza-deoxycytidine), zebularine, isothiocyanates, azacitidine (5-azacytidine), 5-flouro-2′-deoxycytidine, 5,6-dihydro-5-azacytidine and others). Exemplary agents include Aclarubicin, Actinomycin, Alitretinoin, Altretamine, Aminopterin, Aminolevulinic acid, Amrubicin, Amsacrine, Anagrelide, Arsenic trioxide, Asparaginase, Atrasentan, Belotecan, Bexarotene, bendamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

Because some drugs work better together than alone, two or more drugs are often given at the same time. Often, two or more chemotherapy agents are used as combination chemotherapy.

In some embodiments, the additional cancer therapeutic agent is a differentiation agent. Such differentiation agent includes retinoids (such as all-trans-retinoic acid (ATRA), 9-cis retinoic acid, 13-cis-retinoic acid (13-cRA) and 4-hydroxy-phenretinamide (4-HPR)); arsenic trioxide; histone deacetylase inhibitors HDACs (such as azacytidine (Vidaza) and butyrates (e.g., sodium phenylbutyrate)); hybrid polar compounds (such as hexamethylene bisacetamide ((HMBA)); vitamin D; and cytokines (such as colony-stimulating factors including G-CSF and GM-CSF, and interferons).

In some embodiments the additional cancer therapeutic agent is a targeted therapy agent. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors such as Axitinib, Bosutinib, Cediranib, dasatinib, erlotinib, imatinib, gefitinib, lapatinib, Lestaurtinib, Nilotinib, Semaxanib, Sorafenib, Sunitinib, and Vandetanib, and also cyclin dependent kinase inhibitors such as Alvocidib and Seliciclib. Monoclonal antibody therapy is another strategy in which the therapeutic agent is an antibody which specifically binds to a protein on the surface of the cancer cells. Examples include the anti HER2/neu antibody trastuzumab (HERCEPTIN®) typically used in breast cancer, and the anti CD20 antibody rituximab and Tositumomab typically used in a variety of B cell malignancies. Other exemplary antibodies include Cetuximab, Panitumumab, Trastuzumab, Alemtuzumab, Bevacizumab, Edrecolomab, and Gemtuzumab. Exemplary fusion proteins include Aflibercept and Denileukin diftitox. In some embodiments, the targeted therapy can be used in combination with a compound described herein, e.g., a biguanide such as metformin or phenformin, preferably phenformin.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell. An example of such therapy includes BEXXAR®.

In some embodiments, the additional cancer therapeutic agent is an immunotherapy agent. Cancer immunotherapy refers to a diverse set of therapeutic strategies designed to induce the subject's own immune system to fight the tumor. Contemporary methods for generating an immune response against tumors include intravesicular BCG immunotherapy for superficial bladder cancer, and use of interferons and other cytokines to induce an immune response in renal cell carcinoma and melanoma subjects.

Allogeneic hematopoietic stem cell transplantation can be considered a form of immunotherapy, since the donor's immune cells will often attack the tumor in a graft versus tumor effect. In some embodiments, the immunotherapy agents can be used in combination with a compound or composition described herein.

In some embodiments, the additional cancer therapeutic agent is a hormonal therapy agent. The growth of some cancers can be inhibited by providing or blocking certain hormones. Common examples of hormone sensitive tumors include certain types of breast and prostate cancers. Removing or blocking estrogen or testosterone is often an important additional treatment. In certain cancers, administration of hormone agonists, such as progestogens may be therapeutically beneficial. In some embodiments, the hormonal therapy agents can be used in combination with a compound or a composition described herein.

Other possible additional therapeutic modalities include imatinib, gene therapy, peptide and dendritic cell vaccines, synthetic chlorotoxins, and radiolabeled drugs and antibodies.

In one embodiment, the compositions provided herein are used for treatment of AML in combination with an AML induction and consolidation therapy. In one embodiment, the AML induction therapy is a combination of cytarabine and daunorubicin. In one embodiment, the AML induction therapy is a combination of cytarabine and idarubicin.

In one embodiment, the AML consolidation therapy is cytarabine. In one embodiment, the AML consolidation therapy is a combination of mitoxantrone and etoposide.

In one embodiment, the compositions provided herein are used in combination with one or more DNA demethylating agents. In one embodiment, the DNA demethylating agent is a cytidine analog. In certain embodiments, the cytidine analog is azacitidine or 5-aza-2′-deoxycytidine (decitabine). In certain embodiments, the cytidine analog is azacitidine. In certain embodiments, the cytidine analog is 5-aza-2′-deoxycytidine (decitabine). In certain embodiments, the cytidine analog is, for example: 1-β-D-arabinofuranosylcytosine (cytarabine or ara-C); pseudoiso-cytidine (psi ICR); 5-fluoro-2′-deoxycytidine (FCdR); 2′-deoxy-2′,2′-difluorocytidine (gemcitabine); 5-aza-2′-deoxy-2′,2′-difluorocytidine; 5-aza-2′-deoxy-2′-fluorocytidine; 1-β-D-ribofuranosyl-2(1H)-pyrimidinone (zebularine); 2′,3′-dideoxy-5-fluoro-3′-thiacytidine (emtriva); 2′-cyclocytidine (ancitabine); 1-β-D-arabinofuranosyl-5-azacytosine (fazarabine or ara-AC); 6-azacitidine (6-aza-CR); 5,6-dihydro-5-azacitidine (dH-aza-CR); N⁴ pentyloxy-carbonyl-5′-deoxy-5-fluorocytidine (capecitabine); N⁴ octadecyl-cytarabine; or elaidic acid cytarabine. In certain embodiments, the cytidine analogs include any compound which is structurally related to cytidine or deoxycytidine and functionally mimics and/or antagonizes the action of cytidine or deoxycytidine.

In one embodiment, the compositions provided herein are used in combination with azacitidine.

In one embodiment, the compositions provided herein are used in combination with a FLT3 inhibitor. In one embodiment, the FLT3 inhibitor is selected from quizartinib (AC220), sunitinib (SU11248), sorafenib (BAY 43-9006), midostaurin (PKC412), crenolanib (CP-868596), PLX3397, E6201, AKN-028, ponatinib (AP24534), ASP2215, KW-2449, famitinib and DCC-2036.

In one embodiment, the compositions provided herein are used in combination with MEK kinase inhibitor. In one embodiment, the MEK kinase is selected from trametinib, selumetinib, binimetinib, PD-325901, cobimetinib, CI-1040 and PD035901.

In one embodiment, the compositions provided herein are used in combination with a JAK inhibitor. In one embodiment, the compositions provided herein are used in combination with a JAK2 inhibitor. In one embodiment, the JAK2 inhibitor is selected from INCB018424 (ruxolitinib), TG101348, CYT387, AZD1480, SB1518 (pacritinib), XL019, NCB0-16562, NVP-BSK805, R723, hydroxycarbamide, SAR302503, CP-690,550 (tasocitinib) and INCB16562. In one embodiment, the compositions provided herein are used in combination with ruxolitinib.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the methods of use provided herein, may be made without departing from the spirit and scope thereof. Patents, patent publications, and other publications referenced herein are incorporated by reference.

### EXAMPLES

The embodiments described below are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

The following abbreviations are used:

### Abbreviation of Chemicals

THF Tetrahydrofuran

TFE Trifluoroethanol

### Other Abbreviations

AAC Accelerated Aging Conditions (40° C. and 70% RH)

Am Amorphous

API Active Pharmaceutical Ingredient

CF Co-former

CO Experiment ID of the co-crystallization experiments

DSC Differential Scanning Calorimetry

HPLC High-Performance Liquid Chromatography

HR-XRPD High Resolution X-Ray Powder Diffraction

HT-XRPD High Throughput X-Ray Powder Diffraction

MS Mass Spectroscopy

RH Relative Humidity

RT Room Temperature

SDTA Single Differential Thermal Analysis

TGA Thermo Gravimetric Analysis

TGMS Thermo Gravimetric Analysis coupled with Mass Spectroscopy

High Throughput X-Ray Powder Diffraction

HT-XRPD patterns were obtained using the Crystallics T2 high-throughput XRPD set-up. The plates were mounted on a Bruker General Area Detector Diffraction System (GADDS) equipped with a VANTEC-500 gas area detector corrected for intensity and geometric variations. The calibration of the measurement accuracy (peaks position) was performed using NIST SRM1976 standard (Corundum).

Data collection was carried out at room temperature using monochromatic CuKα radiation in the 2 Å region between 1.5° and 41.5°, which is the most distinctive part of the XRPD pattern. The diffraction pattern of each well was collected in two 2θ ranges (1.5°≤2θ≤21.5° for the first frame, and 19.5°≤2θ≤41.5° for the second) with an exposure time of 90s for each frame. No background subtraction or curve smoothing was applied to the XRPD patterns.

The carrier material used during XRPD analysis was transparent to X-rays and contributed only slightly to the background.

High Resolution X-Ray Powder Diffraction

### Measurements:

The powder data were collected on D8 Advance diffractometer using Cu Kα1 radiation (1.54056 Å) with germanium monochromator at Room Temperature. The data were collected from 3 to 41.5° 2θ. Detector scan on solid state Lynx Eye detector was performed using 0.016° per step with different values of sec/step scan speed (see Table 19). The samples were measured in 8 mm long glass capillary with 0.3 mm outer diameter.

### Calculations:

Cell parameters as well as crystal system were obtained using LSI-Index (Coelho, A. A. (2003), J. Appl. Cryst., 36, 86-95; and Coelho, A. A. & Kern, A. (2005), TOPAS. CPD Newsletter, 32, 43-45) indexing program. The space group was selected based on reflections condition as well as density of the crystal. In every sample the co-crystal agent to API ratio was estimated based on integrated proton numbers by 1H NMR as well as possible density of the crystal. The number of molecules in the unit cell was determined using the density of the crystal. The co-crystal agent structures were taken from Cambridge Structural Database (Cambridge Structural Database (2017). https://www.ccdc.cam.ac.uk/structures/?ccdc-check=71ble093cdedc20d09398d7775c3b60e). The cell parameters, purity as well as instrument parameters were refined using Whole Powder Pattern Decomposition method (Pawley, G. S. (1981), J. Appl. Cryst., 14, 357-361). The following criteria of fit were used:

Y_{o,m} and Y_{c,m} are the observed and calculated data, respectively at data point m,

M the number of data points,

P the number of parameters,

wₘ the weighting given to data point m which for counting statistics is given by wₘ=I/σ(Y_{o,m})² where σ(Y_{o,m}) is the error in Y_{o,m},

$${R}_{exp} = \sqrt{\frac{M - P}{\sum {w}_{m} {Y}_{o , m}^{2}}} ; {R}_{wp} = \sqrt{\frac{\sum {{w}_{m} ( {Y}_{o , m} - {Y}_{c , m} )}^{2}}{\sum {w}_{m} {Y}_{o , m}^{2}}} ;$$

$${R}_{p} = \sqrt{\frac{\sum \vert {Y}_{o , m} - {Y}_{c , m} \vert}{\sum {Y}_{o , m}}}$$

$$G O F = c h {i}^{2} = \frac{{R}_{w p}}{{R}_{exp}} = \sqrt{\frac{\sum {{w}_{m} ( {Y}_{o , m} - {Y}_{c , m} )}^{2}}{M - P}}$$

Thermal Analysis

DSC Analysis

Melting properties were obtained from DSC thermograms, recorded with a heat flux DSC822e instrument (Mettler-Toledo GmbH, Switzerland). The DSC822e was calibrated for temperature and enthalpy with a small piece of indium (melting point at 156.6° C.; ΔHf=28.45 J/g). Samples were sealed in standard 40 μL aluminum pans, pin-holed and heated in the DSC from 25° C. to 300° C., at a heating rate of 10° C./min. Dry N₂ gas, at a flow rate of 50 mL/min was used to purge the DSC equipment during the measurement.

TGMS Analysis

Mass loss due to solvent or water loss from the crystals was determined by TGA/SDTA. Monitoring the sample weight, during heating in a TGA/SDTA851e instrument (Mettler-Toledo GmbH, Switzerland), which resulted in a weight vs. temperature curve. The TGA/SDTA851e was calibrated with samples of indium and aluminum. Samples were weighed into 100 μL aluminum crucibles and sealed. The seals were pin-holed and the crucibles heated in the TGA from 25 to 300° C. at a heating rate of 10° C./min. Dry N₂ gas was used for purging.

The gases coming from the TGA samples were analyzed by a mass spectrometer Omnistar GSD 301 T2 (Pfeiffer Vacuum GmbH, Germany). The latter is a quadrupole mass spectrometer, which analyzes masses in the range of 0-200 amu.

HPLC Analytical Method

Method name: S16124_01

HPLC System:

HPLC: Agilent 1200

Detector 1: DAD set at 270 nm

Detector 2: HP1100 LC/MSD in Positive Scan mode

HPLC Conditions:

Auto sampler temp: 20° C.

Column: Waters Sunfire C18 (100×4.6 mm; 3.5 m).

Column temp: 35° C.

Flow cell: 10 mm path

Gradient: Mobile phase A: 10 mM Ammonium acetate

Mobile phase B: Acetonitrile

Flow: 1.0 ml/min

Gradient:

| Time [min]: | Eluent A: | Eluent B: |
|---|---|---|
| 0 | 90% | 10% |
| 1 | 90% | 10% |
| 6 | 10% | 90% |
| 9 | 10% | 90% |
| 10 | 90% | 10% |

Sample:

Concentration: ca. 0.2 mg/ml

Solvent: acetonitrile

Injection volume: 5 μl

The compound integrity is expressed as a peak-area percentage, calculated from the area of each peak in the chromatogram, except the ‘injection peak’, and the total peak-area, as follows:

$$peak area ( \% ) = \frac{peak area}{total area of all peaks} \cdot 100 \%$$

The peak area percentage of the compound of interest is employed as an indication of the purity of the component in the sample.

### Example 1: Compound 1A—Starting Material Characterization

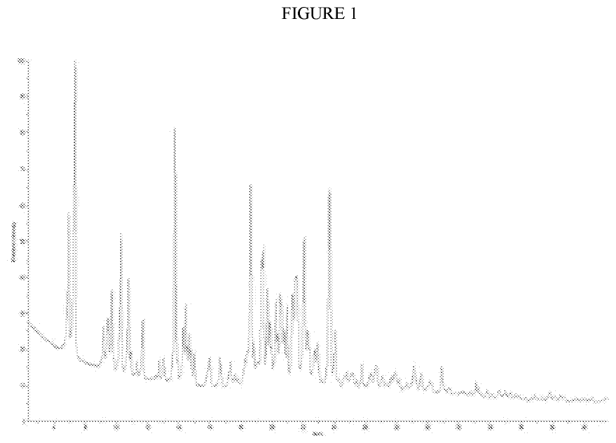

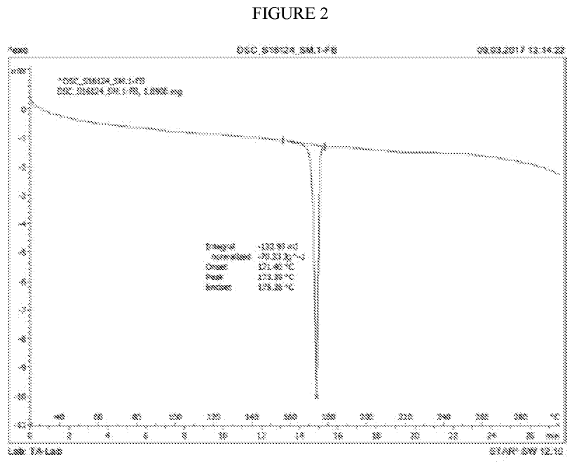

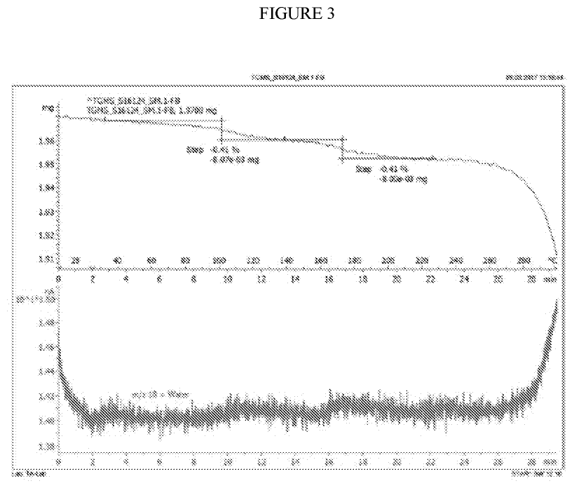

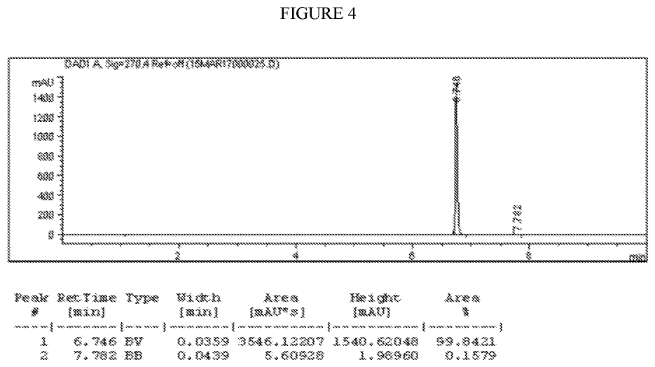

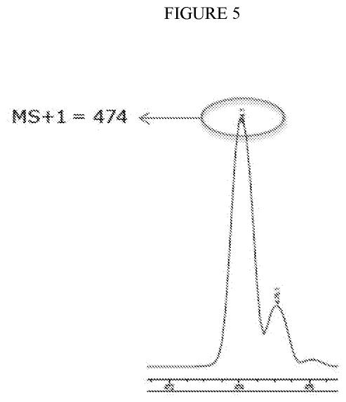

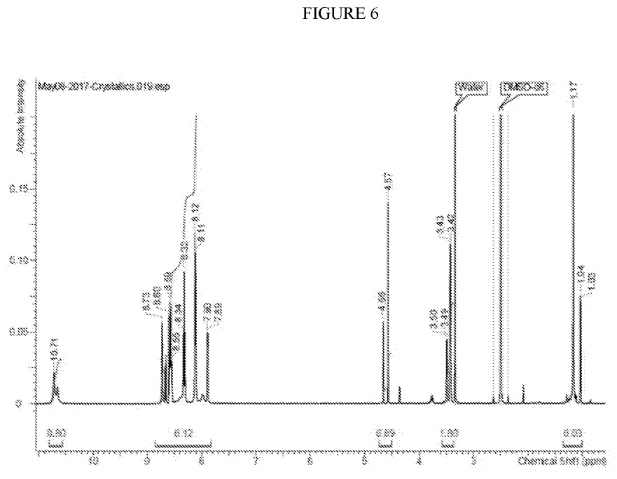

Compound 1A used in the following examples was a crystalline solid which was a mixture of two crystalline phases Form 17 and Form A. The High Resolution XRPD analysis on the starting material is provided in FIG. 1. The material was also characterized by thermal analysis, LCMS and proton NMR. The DSC thermogram indicated a melting point of 173.4° C. (FIG. 2). A mass loss of 0.4% was observed prior to melting, possibly related to residual process solvents. The thermal decomposition occurred above 240° C. (FIG. 3). The HPLC profile and MS data of the starting material are provided in FIGS. 4 and 5, respectively. The main peak in HPLC was observed at a retention time of 6.7 min with a chemical purity of 99.8% (area %). The MS signal confirmed the molecular weight of 473 g/mol corresponding to the molecular weight of Compound 1A. The ¹H-NMR is of the starting material is provided in FIG. 6.

### Example 2: Solubility of Compound 1A

A quantitative (24-hour shake flask) thermodynamic solubility determination was performed on Compound 1A to aid in the selection of the screening solvents.

To assist in the solvent selection for the screening experiments a solubility study was performed. About 30 mg of the compound was used to prepare suspensions in various solvents. After equilibration at room temperature for 24 hours, the solids and liquids were separated by filtration. The concentration of solute was determined by HPLC analysis. The results of the solubility determination are provided in Table 1.

In 1,4-dioxane and water, conversion to Form G and Form 1 were observed, respectively. The forms obtained from the solubility experiments were Form 17, Form A and Form G, and Form 1.

TABLE 1
Solubility of Compound 1A

| | Solubility | |
|---|---|---|
| Solvent | (mg/mL) | Form |
| Methanol | >463 | Not determined |
| Tetrahydrofuran | >429 | Not determined |
| Ethanol | >408 | Not determined |
| 2-Propanol | 229 | Form 17 |
| Acetone | 209 | Form A |
| Ethyl acetate | 127 | Form A |
| Chloroform | 70.6 | Form 17 |
| 1,4-Dioxane | 69.2 | Form G |
| Acetonitrile | 14.7 | Form 17 + Form A |
| Water | <0.1 | Form 1 |

### Example 3: Co-Crystallization Screen

The co-crystal screen on Compound 1A was carried out using three different methods: A) co-crystallization from the melt, B) solvent-drop assisted sonication and C) co-crystallization from a saturated solution. The solvent selection for the solvent mediated methods was based on the results of the solubility determination. Even though in some solvents solvates or hydrates were obtained, these solvents were not excluded from the selection as co-crystallization could still take place if the supramolecular interaction between the co-former and Compound 1A prevails over the interaction between the solvent and the API. The 10 co-formers that were used for the screen are listed in Table 2.

TABLE 2
Co-formers used for the co-crystallization screen.
The three-letter abbreviation was used for the XRPD
classification and naming of potential co-crystals.

| Co-crystal former | Abbreviation | Co-crystal former | Abbreviation |
|---|---|---|---|
| Fumaric acid | Fum | Uracil | Ura |
| Succinic acid | Suc | Saccharin | Sac |
| Asparagine | Asp | Citric acid | Cit |
| Nicotinamide | Nic | Lactamide | Lac |
| Benzoic acid | Ben | 4-Hydroxybenzamide | Hbe |

A) Co-Crystallization from the Melt

For the co-melting studies, physical mixtures with a stoichiometric ratio of Compound 1A and co-former of (1:1) were weighed into 1.8 mL glass vials. The physical mixtures were ground manually to obtain a homogenous mixture. About 2 mg of the mixture were transferred to a DSC crucible and were heated up to 300° C. in the DSC apparatus with 10° C./min. The thermal events were integrated. Experiments CO1, C04, C05, C09 and CO10 were re-measured by DSC. CO1 was heated to 200° C., C04 was heated to 150° C., C05 was heated to 110° C., C09 was heated to 130° C. and CO10 to 170° C. After cooling the sample to room temperature, the solid was recovered from the crucible and was analyzed by HT-XRPD. Experiment CO1 was repeated with a physical mixture of 1:0.5 (Exp C0123). The experimental details and results are described in Table 3.

TABLE 3
Experimental conditions and results of the co-crystallization by melt experiments

| | | Mass | | Ratio | | | | |
|---|---|---|---|---|---|---|---|---|
| | | Compound | Mass CF | FB:CF | Melt 1 | Recryst | Melt 2 | |
| Exp ID | Co-former (CF) | 1A (mg) | (mg) | (1:x) | (° C.) | (° C.) | (° C.) | Form |
| SM | — | — | — | — | 173 | — | — | — |
| CO1 | Fumaric acid | 21.9 | 5.4 | 1 | 170 | 173 (broad) | 212 | Fum1 + Fum0 |
| CO123 | Fumaric acid | 16.3 | 2.2 | 0.6 | 171 | 184 (broad) | 212 | Fum1 |
| CO2 | Succinic acid | 20.4 | 5.1 | 1 | 168 | — | 179 | — |
| CO3 | L-(+)-Asparagine | 19.6 | 5.5 | 1 | 173 | — | 234 | — |
| CO4 | Nicotinamide | 21.4 | 5.5 | 1 | 129 | — | 187 | Nic1 |
| CO5 | Benzoic Acid | 20.3 | 5.4 | 1 | 77 | 98 | 151 | Ben1 |
| CO6 | Uracil | 21.6 | 5.1 | 1 | 170 | — | — | — |
| CO7 | Saccharin | 19.7 | 7.8 | 1 | 150 | — | — | — |
| CO8 | Citric acid | 21.9 | 8.9 | 1 | 149 | — | — | — |
| CO9 | Lactamide | 22.8 | 4.5 | 1 | 78 | 101 | 138 | Lac1 |
| CO10 | 4-Hydroxybenzamide | 19.5 | 5.8 | 1 | 141 | 160 | 177 | Hbe1 |

B) Co-Crystallization by Sonication

A solution of Compound 1A in tetrahydrofuran (60 mg/mL) was liquid dosed into four 8-mL glass vials per co-former used. An equimolar amount of co-former was added from an aqueous solution. Uracil was added as a solid, as it is insoluble in most solvents. The solutions were subjected to a freeze drying cycle. The obtained solids of one set of the experiments were harvested and analyzed by HT-XRPD for reference purpose. The experimental details and results are reported in Table 4.

To the other three sets of vials solvent was added drop-wise (10 μL per drop) until a paste-like material was obtained. This paste was sonicated for 10 minutes at room temperature. The samples were dried at 30° C. under vacuum (10 mbar) for 24 hours and analyzed by HT-XRPD. The experimental details and results are reported in Table 5.

All solids were exposed to accelerated aging conditions (40° C./70% RH) for two days and re-analyzed by HT-XRPD.

Hits of co-crystals were analyzed by TGMS to confirm if the novel forms contained any solvent. The samples with low amounts of residual solvent were further characterized by DSC, HPLC and 1H-NMR.

TABLE 4
Experimental conditions and results of the samples from freeze drying. The forms are designated
by the abbreviation of the co-former followed by a number, i.e. Fum1 for a unique pattern
with fumaric acid, ‘Lc’ stands for low crystalline, ‘Am’ means amorphous

| | | Mass | Mass | Ratio | | | |
|---|---|---|---|---|---|---|---|
| | | FB | CF | FB:CF | | | |
| Exp ID | Co-former (CF) | (mg) | (mg) | (1:x) | Solvent | Form | After AAC |
| CO11 | Fumaric acid | 30.0 | 7.4 | 1.0 | Tetrahydrofuran | Lc | Fum1 |
| CO12 | Succinic acid | 30.0 | 7.6 | 1.0 | Tetrahydrofuran | Compound 1A, | Compound 1A, |
| | | | | | | Form 1 | Form 1 |
| CO13 | L-(+)-Asparagine | 30.0 | 8.5 | 1.0 | Tetrahydrofuran | Am | Asp0-1 + |
| | | | | | | | Compound 1A, |
| | | | | | | | Form 1 Lc |
| CO14 | Nicotinamide | 30.0 | 7.7 | 1.0 | Tetrahydrofuran | Nic3 lc | Nic1 + Nic0 |
| CO15 | Benzoic Acid | 30.0 | 7.8 | 1.0 | Tetrahydrofuran | Ben1 | Ben1 |
| CO16 | Uracil | 30.0 | 7.9 | 1.1 | Tetrahydrofuran | Ura0 | Compound 1A, |
| | | | | | | | Form 1 + Ura0 |
| CO17 | Saccharin | 30.0 | 11.9 | 1.0 | Tetrahydrofuran | Sac1 | Sac1 |
| CO18 | Citric acid | 30.0 | 12.2 | 1.0 | Tetrahydrofuran | Am | Am |
| | | | | | | | deliquescent |
| CO19 | Lactamide | 30.0 | 5.8 | 1.0 | Tetrahydrofuran | Am | Lac4 |
| CO20 | 4-Hydroxybenzamide | 30.0 | 8.9 | 1.0 | Tetrahydrofuran | Hbe2 | Hbe1 |
| CO120 | None | 29.6 | — | — | Tetrahydrofuran | Am | Compound 1A, |
| | | | | | | | Form 1 |

TABLE 5
Experimental conditions and results of the co-sonication experiments. The forms are designated
by the abbreviation of the co-former followed by a number, i .e. Fum1 for a unique pattern
with fumaric acid, ‘Lc’ stands for low crystalline, ‘Am’ means amorphous.

| | | Mass | Mass | Ratio | | | |
|---|---|---|---|---|---|---|---|
| | | FB | CF | FB:CF | | | |
| Exp ID | Co-former (CF) | (mg) | (mg) | (1:x) | Solvent | Form | After AAC |
| CO21 | Fumaric acid | 30.0 | 7.4 | 1.0 | Acetonitrile | Fum0 + Fum1 | Fum0 + Fum1 |
| CO22 | Succinic acid | 30.0 | 7.6 | 1.0 | Acetonitrile | Compound 1A, | Compound 1A, |
| | | | | | | Form A + | Form 1 + |
| | | | | | | trace Suc1 | trace Suc1 |
| CO23 | L-(+)-Asparagine | 30.0 | 8.5 | 1.0 | Acetonitrile | Compound 1A, | Compound 1A, |
| | | | | | | Form A | Form A |
| CO24 | Nicotinamide | 30.0 | 7.7 | 1.0 | Acetonitrile | Nic0 + Nic1 | Nic0 + Nic1 |
| CO25 | Benzoic Acid | 30.0 | 7.8 | 1.0 | Acetonitrile | Ben1 | Ben1 |
| CO26 | Uracil | 30.0 | 7.9 | 1.1 | Acetonitrile | Compound 1A, | Compound 1A, |
| | | | | | | Form A + Ura0 | Form A + Ura0 |
| CO27 | Saccharin | 30.0 | 11.9 | 1.0 | Acetonitrile | Compound 1A, | Compound 1A, |
| | | | | | | Form A + Sac1 | Form A + Sac1 |
| CO28 | Citric acid | 30.0 | 12.2 | 1.0 | Acetonitrile | Cit1 | Cit1a |
| CO29 | Lactamide | 30.0 | 5.8 | 1.0 | Acetonitrile | Lac2 | Lac0 + |
| | | | | | | | Lac2 + Lac3 |
| CO30 | 4-Hydroxybenzamide | 30.0 | 8.9 | 1.0 | Acetonitrile | Hbe1 + Hbe2 | Hbe1 |
| CO32 | Fumaric acid | 29.6 | 7.3 | 1.0 | Ethyl acetate | Fum0 + Fum1 | Fum0 + Fum1 |
| CO33 | Succinic acid | 29.6 | 7.5 | 1.0 | Ethyl acetate | Compound 1A, | Compound 1A, |
| | | | | | | Form 1 | Form 1 |
| CO34 | L-(+)-Asparagine | 29.6 | 8.4 | 1.0 | Ethyl acetate | FB2b | Asp0-1 + |
| | | | | | | | Compound 1A, |
| | | | | | | | Form A |
| CO35 | Nicotinamide | 29.6 | 7.6 | 1.0 | Ethyl acetate | FB2b | Compound 1A, |
| | | | | | | | Form A |
| CO36 | Benzoic Acid | 29.6 | 7.7 | 1.0 | Ethyl acetate | Ben1 | Ben0 + Ben1 |
| CO37 | Uracil | 29.6 | 6.8 | 1.0 | Ethyl acetate | FB2b Ura0 | Compound 1A, |
| | | | | | | | Form A + Ura0 |
| CO38 | Saccharin | 29.6 | 11.7 | 1.0 | Ethyl acetate | FB2b + Sac1 | Compound 1A, |
| | | | | | | | Form A + Sac1 |
| CO39 | Citric acid | 29.6 | 12 | 1.0 | Ethyl acetate | Cit1 | Cit4 |
| CO40 | Lactamide | 29.6 | 5.7 | 1.0 | Ethyl acetate | Lac2 | Lac0 + |
| | | | | | | | Lac2 + Lac3 |
| CO41 | 4-Hydroxybenzamide | 29.6 | 8.8 | 1.0 | Ethyl acetate | Hbe1 + Hbe2 | Hbe1 |
| CO42 | Fumaric acid | 29.6 | 7.3 | 1.0 | Chloroform | Fum0 + Fum1 | Fum0 + Fum1 |
| CO43 | Succinic acid | 29.6 | 7.5 | 1.0 | Chloroform | Compound 1A, | Compound 1A, |
| | | | | | | Form 1 | Form 1 + Suc1 |
| CO44 | L-(+)-Asparagine | 29.6 | 8.4 | 1.0 | Chloroform | Lc | Asp0-1 + |
| | | | | | | | Compound 1A, |
| | | | | | | | Form 1 |
| CO45 | Nicotinamide | 29.6 | 7.6 | 1.0 | Chloroform | Am | Compound 1A, |
| | | | | | | | Form 1 |
| CO46 | Benzoic Acid | 29.6 | 7.7 | 1.0 | Chloroform | Ben1 | Ben1 |
| CO47 | Uracil | 29.6 | 7.5 | 1.1 | Chloroform | Ura0 | Compound 1A, |
| | | | | | | | Form 1 + Ura0 |
| CO48 | Saccharin | 29.6 | 11.7 | 1.0 | Chloroform | Compound 1A, | Compound 1A, |
| | | | | | | Form 1 + Sac1 | Form 1 + Sac1 |
| CO49 | Citric acid | 29.6 | 12 | 1.0 | Chloroform | Cit2 | Cit4 |
| CO50 | Lactamide | 29.6 | 5.7 | 1.0 | Chloroform | Am | Lac0 + Lac3 |
| CO51 | 4-Hydroxybenzamide | 29.6 | 8.8 | 1.0 | Chloroform | Hbe1 + Hbe2 | Hbe1 |
| CO31 | None | 30.0 | — | — | Acetonitrile | Compound 1A, | Compound 1A, |
| | | | | | | Form A | Form A |
| CO121 | None | 29.6 | | | Ethyl acetate | FB2b | Compound 1A, |
| | | | | | | | Form 17 + |
| | | | | | | | Compound 1A, |
| | | | | | | | Form A |
| CO122 | None | 30.0 | — | — | Chloroform | Compound 1A, | Compound 1A, |
| | | | | | | Form A + | Form 1 |
| | | | | | | Compound 1A, | |
| | | | | | | Form 1 | |

C) Co-Crystallization from Saturated Solution

Close to saturated solutions of Compound 1A were prepared in six solvents (mixtures) as shown in Table 6. If necessary the mixtures were heated to 50° C. for 15 min to ensure complete dissolution. Aliquots of the stock solutions were divided over eleven 1.8 mL glass vials. To ten of the vials, solid co-formers were added until precipitation was observed. The co-former was added until it did not dissolve anymore or until 4 molar equivalents had been reached. The mixtures were left stirring overnight at room temperature. Subsequently, the solids and liquids were separated. The solids were dried under vacuum (10 mbar) at 30° C. for at least 24 hours. The mother liquors were allowed to evaporate slowly under ambient conditions. The obtained solids were analyzed by HT-XRPD and re-analyzed after exposure to accelerated aging conditions (40° C./70% RH) for two days.

Hits of co-crystals were analyzed by TGMS to confirm if the novel forms contained solvent. The samples with low amounts of residual solvent were further characterized by DSC, HPLC and 1H-NMR.

Table 7 provides experimental conditions and results of the co-crystallization experiments from saturated API solution. Table 8 provides crystal parameters and measurement conditions of HR-XRPD data for the co-crystals that could be indexed.

TABLE 6
Saturated solutions of Compound 1A were prepared
in 6 solvent (mixtures). The stock solutions
were divided over a set of 11 vials

| | Mass | | Volume per | Mass per |
|---|---|---|---|---|
| | Compound | Volume | experiment | experiment |
| Solvent | 1A(mg) | (mL) | (μL) | (mg) |
| Ethyl acetate | 330.8 | 3.0 | 270 | 29.8 |
| Acetone/Water | 328.7 | 1.5 | 125 | 27.4 |
| (90/10) | | | | |
| Tetrahydrofuran/ | 334.2 | 1.1 | 100 | 30.4 |
| Water (85/15) | | | | |
| 1,4-Dioxane | 330.8 | 4.5 | 400 | 29.4 |
| Ethanol/Water | 332.8 | 4.5 | 400 | 29.6 |
| (83/17) | | | | |
| Acetonitrile | 338.8 | 10.0 | 900 | 30.4 |

TABLE 7
Experimental conditions and results of the co-crystallization experiments from saturated API solution. The neat co-formers
are designated by the abbreviation followed by ‘0’. The co-crystals are designated by the abbreviation of the co-former
followed by a number, i.e. Fum1 for a unique pattern with fumaric acid, ‘Lc’ stands for low crystalline, ‘Am’ means amorphous.

| | | | | Ratio | | | | |
|---|---|---|---|---|---|---|---|---|
| | | | CF | FB:CF | Solid phase | | Liquid phase | |
| Exp ID | Co-former (CF) | Solvent | (mg) | (1:x) | | AAC | | AAC |
| CO54 | Fumaric acid | Ethyl acetate | 5.4 | 0.7 | Fum1 | Fum1 + | No solids | — |
| | | | | | | trace Fum0 | | |
| CO55 | Succinic acid | Ethyl acetate | 6.3 | 0.8 | Suc1 + | Suc1 + | Suc0 + | Suc0 + |
| | | | | | trace Suc0 | trace Suc0 | Suc1 + Suc2 | Suc1 + Suc2 |
| CO56 | L-(+)-Asparagine | Ethyl acetate | 3.6 | 0.4 | Asp0 | Asp0 | Am | Compound 1A, |
| | | | | | | | | Form 1 |
| CO57 | Nicotinamide | Ethyl acetate | 8.0 | 1.0 | Nic1 | Nic1 | No solids | — |
| CO58 | Benzoic Acid | Ethyl acetate | 55.5 | 7.1 | Ben0 + Ben1 | Ben0 + Ben1 | Ben0 + | Ben0 + Ben1 |
| | | | | | | | trace Ben1 | |
| CO59 | Uracil | Ethyl acetate | 2.3 | 0.3 | No solids | — | Am | Compound 1A, |
| | | | | | | | | Form 1 |
| CO60 | Saccharin | Ethyl acetate | 16.6 | 1.4 | Sac1 | Sac1 | Sac1 | Sac1 + trace |
| | | | | | | | | Compound 1A, |
| | | | | | | | | Form 1 |
| CO61 | Citric acid | Ethyl acetate | 9.7 | 0.8 | Cit3 | Cit3 | No solids | — |
| CO62 | Lactamide | Ethyl acetate | 10.2 | 1.8 | Lac0 + Lac3 | Lac0 + Lac3 | Lac0 + Lac3 | Lac0 + Lac3 |
| CO63 | 4-Hydroxybenzamide | Ethyl acetate | 10.9 | 12 | Hbe1 | Hbe1 | Hbe0 + Hbe1 | Hbe0 + Hbe1 |
| CO64 | Fumaric acid | Acetone/Water | 10.3 | 1.5 | Fum0 + Fum1 | Fum0 + Fum1 | No solids | |
| | | (90/10) | | | | | | |
| CO65 | Succinic acid | Acetone/Water | 23.1 | 3.3 | Suc0 + Suc1 | Suc0 + Suc1 | Suc0 + Suc1 | Suc0 + Suc1 |
| | | (90/10) | | | | | | |
| CO66 | L-(+)-Asparagine | Acetone/Water | 4.6 | 0.6 | Compound 1A, | Compound 1A, | Compound 1A, | Compound 1A, |
| | | (90/10) | | | Form 1 + Asp0-1 | Form 1 + Asp0-1 | Form 1 | Form 1 |
| CO67 | Nicotinamide | Acetone/Water | 29.5 | 4.2 | Nic0 + Nic1 | Nic0 + Nic1 | No solids | — |
| | | (90/10) | | | | | | |
| CO68 | Benzoic Acid | Acetone/Water | 46.9 | 6.6 | Ben0 + Ben1 | Ben0 + Ben1 | Ben0 + | Ben0 + Ben1 |
| | | (90/10) | | | | | trace Ben1 | |
| CO69 | Uracil | Acetone/Water | 2.9 | 0.4 | Compound 1A, | Compound 1A, | Compound 1A, | Compound 1A, |
| | | (90/10) | | | Form 1 + Ura0 | Form 1 + Ura0 | Form 1 | Form 1 |
| CO70 | Saccharin | Acetone/Water | 21.5 | 2.0 | Sac1 | Sac1 | Sac1 | Sac1 |
| | | (90/10) | | | | | | |
| CO71 | Citric acid | Acetone/Water | 49.8 | 4.5 | Cit0 + Cit1 | Cit0 + Cit1 | Cit0 | Cit0 |
| | | (90/10) | | | | | | |
| CO72 | Lactamide | Acetone/Water | 25.8 | 4.8 | Lac0 + Lac3 | Lac0 + Lac3 | Lac0 + Lac3 | Lac0 + Lac3 |
| | | (90/10) | | | | | | |
| CO73 | 4-Hydroxybenzamide | Acetone/Water | 38.3 | 4.7 | Hbe0 + Hbe3 | Hbe3 | No solids | — |
| | | (90/10) | | | | | | |
| CO74 | Fumaric acid | THF/Water | 29.8 | 4.0 | Fum0 + Fum1 | Fum0 + Fum1 | Fum1 | Fum0 + Fum1 |
| | | (85/15) | | | | | | |
| CO75 | Succinic acid | THF/Water | 43.8 | 5.7 | Suc0 | Suc0 | Suc0 + Suc2 | Suc0 + Suc2 |
| | | (85/15) | | | | | | |
| CO76 | L-(+)-Asparagine | THF/Water | 6.4 | 0.7 | Asp0-1 | Asp0-1 | Compound 1A, | Compound 1A, |
| | | (85/15) | | | | | Form 1 | Form 1 |
| CO77 | Nicotinamide | THF/Water | 41.8 | 5.3 | Nic0 + Nic1 | Nic0 + Nic1 | No solids | — |
| | | (85/15) | | | | | | |
| CO78 | Benzoic Acid | THF/Water | 111.2 | 14.0 | Ben0 + Ben1 | Ben0 + Ben1 | Ben0 + Ben1 | Ben0 + Ben1 |
| | | (85/15) | | | | | | |
| CO79 | Uracil | THF/Water | 2.9 | 0.4 | Ura0 | Ura0 | Compound 1A, | Compound 1A, |
| | | (85/15) | | | | | Form 1 | Form 1 |
| CO80 | Saccharin | THF/Water | 54.3 | 4.5 | Sac0 | Sac0 | Sac0 | Compound 1A, |
| | | (85/15) | | | | | | Form 1 + Sac0 |
| CO81 | Citric acid | THF/Water | 64.6 | 5.2 | Cit0 + Cit4 | Cit0 + Cit4 | Cit0 | Cit0 + Cit5 |
| | | (85/15) | | | | | | |
| CO82 | Lactamide | THF/Water | 58.8 | 10 | Lac0 + Lac3 | Lac0 + Lac3 | Lac0 + | Lac0 + |
| | | (85/15) | | | | | trace Lac3 | trace Lac3 |
| CO83 | 4-Hydroxybenzamide | THF/Water | 37.0 | 4.1 | FB3 or FB7 + | FB3 or FB7 + | No solids | — |
| | | (85/15) | | | Hbe1 | Hbe1 | | |
| CO84 | Fumaric acid | 1,4-Dioxane | 16.7 | 2.3 | Fum0 + Fum1 | Fum0 + Fum1 | Fum0 + Fum1 | Fum0 + Fum1 |
| CO85 | Succinic acid | 1,4-Dioxane | 36.9 | 5.0 | Suc0 | Suc0 | Suc0 + Suc1 | Suc0 + Suc1 |
| CO86 | L-(+)-Asparagine | 1,4-Dioxane | 5.9 | 0.7 | Asp0 | Asp0 | Compound 1A, | Compound 1A, |
| | | | | | | | Form G | Form G + |
| | | | | | | | | Compound 1A, |
| | | | | | | | | Form 1 |
| CO87 | Nicotinamide | 1,4-Dioxane | 6.3 | 0.8 | Nic3 | Nic3 | Compound 1A, | Compound 1A, |
| | | | | | | | Form G + Nic3 | Form G + |
| | | | | | | | | Compound 1A, |
| | | | | | | | | Form 1 + Nic3 |
| CO88 | Benzoic Acid | 1,4-Dioxane | 60.1 | 7.8 | No solids | — | Ben0 + Ben1 | Ben0 + Ben1 |
| CO89 | Uracil | 1,4-Dioxane | 6.4 | 0.9 | Compound 1A, | Compound 1A, | Compound 1A, | Compound 1A, |
| | | | | | Form G + Ura0 | Form G + Ura0 | Form G | Form G + |
| | | | | | | | | Compound 1A, |
| | | | | | | | | Form 1 |
| CO90 | Saccharin | 1,4-Dioxane | 50.5 | 4.3 | No solids | — | Compound 1A, | Compound 1A, |
| | | | | | | | Form G + Sac0 | Form G + |
| | | | | | | | | Compound 1A, |
| | | | | | | | | Form 1 + Sac0 |
| CO91 | Citric acid | 1,4-Dioxane | 48.1 | 4.0 | No solids | — | Cit4 + peaks | Cit0 + Cit4 |
| CO92 | Lactamide | 1,4-Dioxane | 26.9 | 4.7 | Lac0 + Lac3 | Lac0 + Lac3 | Lac0 + Lac3 | Lac0 + Lac3 |
| CO93 | 4-Hydroxybenzamide | 1,4-Dioxane | 8.7 | 1.0 | Hbe0-2 | Hbe0 + | Compound 1A, | Compound 1A, |
| | | | | | | trace Hbe1 | Form G | Form G + |
| | | | | | | | | Compound 1A, |
| | | | | | | | | Form 1 |
| CO94 | Fumaric acid | Ethanol/Water | 22.9 | 3.2 | Fum0 + Fum1 | Fum0 + Fum1 | Fum0 | Fum0 |
| | | (83/17) | | | | | | |
| CO95 | Succinic acid | Ethanol/Water | 46.4 | 6.2 | Suc0 + Suc1 | Suc0 + Suc1 | Suc0 + Suc1 | Suc0 + Suc1 |
| | | (83/17) | | | | | | |
| CO96 | L-(+)-Asparagine | Ethanol/Water | 3.9 | 0.5 | Asp0 + | Asp0 + | Compound 1A, | Compound 1A, |
| | | (83/17) | | | Compound 1A, | Compound 1A, | Form 1 | Form 1 |
| | | | | | Form 1 | Form 1 | | |
| CO97 | Nicotinamide | Ethanol/Water | 67.7 | 8.9 | Nic0 + Nic2 | Nic0 + Nic2 | Nic0 | Nic0 |
| | | (83/17) | | | | | | |
| CO98 | Benzoic Acid | Ethanol/Water | 76.5 | 9.9 | Ben0 + Ben1 | Ben0 + Ben1 | Ben0 + Ben1 | Ben0 + Ben1 |
| | | (83/17) | | | | | | |
| CO99 | Uracil | Ethanol/Water | 3.1 | 0.4 | Compound 1A, | Compound 1A, | Compound 1A, | Compound 1A, |
| | | (83/17) | | | Form 1 + Ura0 | Form 1 + Ura0 | Form 1 | Form 1 |
| CO100 | Saccharin | Ethanol/Water | 16.7 | 1.4 | Sac1 | Sac1 | Sac1 | Sac1 |
| | | (83/17) | | | | | | |
| CO101 | Citric acid | Ethanol/Water | 55.1 | 4.6 | Cit4 | Cit4 | Cit0 + Cit5 | Cit0 + Cit5 |
| | | (83/17) | | | | | | |
| CO102 | Lactamide | Ethanol/Water | 74.0 | 12.9 | Lac0 + Lac3 | Lac0 + Lac3 | Lac0 + | Lac0 + |
| | | (83/17) | | | | | trace Lac3 | trace Lac3 |
| CO103 | 4-Hydroxybenzamide | Ethanol/Water | 38.0 | 4.3 | Hbe0 + | Hbe0 + | Hbe0-1 | Hbe0-1 |
| | | (83/17) | | | Hbe1 + Hbe2 | Hbe1 + Hbe2 | | |
| CO104 | Fumaric acid | Acetonitrile | 15.9 | 2.1 | Fum1 | Fum1 | Fum0 + Fum1 | Fum0 + Fum1 |
| CO105 | Succinic acid | Acetonitrile | 16.1 | 2.1 | Suc1 | Suc1 | Suc1 + Suc0-1 | Suc0 + |
| | | | | | | | | Suc1 + Suc2 |
| CO106 | L-(+)-Asparagine | Acetonitrile | 4.6 | 0.5 | Asp0 + | Asp0 + | Compound 1A, | Compound 1A, |
| | | | | | Compound 1A, | Compound 1A, | Form 1 | Form 1 |
| | | | | | Form 1 | Form 1 | | |
| CO107 | Nicotinamide | Acetonitrile | 37.5 | 4.8 | Nic1 + | Nic1 + | Nic0 | Nic0 |
| | | | | | trace Nic0 | trace Nic0 | | |
| CO108 | Benzoic Acid | Acetonitrile | 76.3 | 9.6 | Ben0 + Ben1 | Ben0 + Ben1 | Ben0 + Ben1 | Ben0 + Ben1 |
| CO109 | Uracil | Acetonitrile | 4.6 | 0.6 | Ura1 | Ura1 | Compound 1A, | Compound 1A, |
| | | | | | | | Form 17 + | Form 17 + |
| | | | | | | | Compound 1A, | Compound 1A, |
| | | | | | | | Form A | Form A |
| CO110 | Saccharin | Acetonitrile | 56.2 | 4.7 | Sac1 + | Sac1 + | Sac1 + | Sac0 + Sac1 |
| | | | | | trace Sac0 | trace Sac0 | trace Sac0 | |
| CO111 | Citric acid | Acetonitrile | 72.9 | 5.9 | Cit4 | Cit4 | Cit0 | Cit0 |
| CO112 | Lactamide | Acetonitrile | 66.2 | 11.2 | Lac0 + Lac3 | Lac0 + Lac3 | Lac0 + | Lac0 + |
| | | | | | | | trace Lac3 | trace Lac3 |
| CO113 | 4-Hydroxybenzamide | Acetonitrile | 27.4 | 3.0 | Hbe1 | Hbe1 | Hbe0 + Hbe1 + | Hbe0 + Hbe1 |
| | | | | | | | Hbe3 | |
| CO114 | None | Ethyl acetate | — | — | No solids | — | FB2b | FB2b + |
| | | | | | | | | Compound 1A, |
| | | | | | | | | Form 1 |
| CO115 | None | Acetone/Water | — | — | Compound 1A, | Compound 1A, | Compound 1A, | Compound 1A, |
| | | (90/10) | | | Form 1 | Form 1 | Form 1 | Form 1 |
| CO116 | None | THF/Water | — | — | No solids | — | Compound 1A, | Compound 1A, |
| | | (85/15) | | | | | Form 2 | Form 1 + |
| | | | | | | | | Compound 1A, |
| | | | | | | | | Form 2 |
| CO117 | None | 1,4-Dioxane | — | — | No solids | — | Compound 1A, | Compound 1A, |
| | | | | | | | Form G | Form G + |
| | | | | | | | | Compound 1A, |
| | | | | | | | | Form 1 |
| CO118 | None | Ethanol/Water | — | — | No solids | — | Compound 1A, | Compound 1A, |
| | | (83/17) | | | | | Form 1 | Form 1 |
| CO119 | None | Acetonitrile | — | — | No solids | — | Compound 1A, | Compound 1A, |
| | | | | | | | Form 17 + | Form 17 + |
| | | | | | | | Compound 1A, | Compound 1A, |
| | | | | | | | Form 1 | Form 1 |

TABLE 8
Crystal parameters and measurement conditions of HR-XRPD data for the co-crystals that could be indexed.

| | Sac1 | | Fum1 | | Suc1 | | Cit3 | |
|---|---|---|---|---|---|---|---|---|
| Experiment ID | (CO70) | | (CO54) | | (CO105) | | (CO61) | |
| Empirical formula¹ | C₁₉H₁₇F₆N₇O•2(C₇H₅NO₃S) | | C₁₉H₁₇F₆N₇O•0.5(C₄H₄O₄) | | C₁₉H₁₇F₆N₇O•0.5(C₄H₆O₄) | | C₁₉H₁₇F₆N₇O•C₆H₈O₈ | |
| Fw | 807.68 | | 543.43 | | 545.44 | | 665.50 | |
| T [K] | 296(2)K | | 296(2)K | | 296(2)K | | 296(2)K | |
| λ [Å] | 1.54056 | | 1.54056 | | 1.54056 | | 1.54056 | |
| Crystal system | Triclinic | | Monoclinic | | Monoclinic | | Monoclinic | |
| Proposed | P-1 | | P2₁/c | | P2₁/c | | P2₁/c | |
| Space group | | | | | | | | |
| Unit cell | | | | | | | | |
| dimensions | | | | | | | | |
| a [Å] | 7.97929 | (11) | 5.5057 | (2) | 5.48946 | (10) | 5.44695 | (9) |
| b [Å] | 14.2463 | (2) | 21.7473 | (7) | 21.8490 | (4) | 27.1953 | (6) |
| c [Å] | 16.9498 | (3) | 19.3686 | (6) | 19.3991 | (4) | 20.6852 | (4) |
| α [°] | 81.4795 | (9) | — | | — | | — | |
| β [°] | 75.5225 | (9) | 90.3194 | (13) | 90.1230 | (8) | 106.156 | (2) |
| γ [°] | 74.727 | (2) | — | | — | | — | |
| V [Å³] | 1792.70 | (5) | 2319 08 | (12) | 2326.37 | (7) | 2943.12 | (10) |
| Z (Z′) | 2 | (1) | 4 | (1) | 4 | (1) | 4 | (1) |

| | | | | |
|---|---|---|---|---|
| D_{c} [g/cm³] | 1.473 | 1.556 | 1.557 | 1.502 |
| Cap. size [mm²] | 03 × 8 | 0.3 × 8 | 0.3 × 8 | 0.3 × 8 |
| 2θ Step size [°] | 0.16 | 0.16 | 0.16 | 0.16 |
| No of steps | 2441 | 2441 | 2441 | 2441 |
| Time per step [s] | 13 | 2 | 10 | 10 |
| 2θ range[°] | 3-41.5 | 3-41.5 | 3-41.5 | 3-41.5 |
| Rexp | 0.75 | 2.13 | 0.79 | 0.77 |
| R_{wp} | 2.22 | 2.74 | 1.54 | 1.58 |
| Rₚ | 1.46 | 2.09 | 1.08 | 1.20 |
| GOF | 2.97 | 1.29 | 1.95 | 2.04 |
| R_{Brag} | 0.11 | 0.21 | 0.06 | 0.07 |
| Impurities [%] | 3.5 | 2 (α form of | 3.5 (α form of | Not detected |
| | (Saccharin) | Fumaric acid) | Succinic acid) | |
| | | | and 0.5% of | |
| | | | other phase | |

| | Cit4 | | Ben1 | | Nic1 | | Hbe1 | |
|---|---|---|---|---|---|---|---|---|
| Experiment ID | (CO111) | | (CO15) | | (CO57) | | (CO63) | |
| Empirical formula¹ | C₁₉H₁₇F₆N₇O•C₆H₈O₈ | | C₁₉H₁₇F₆N₇O•C₇H₅O₂ | | C₁₉H₁₇F₆N₇O•0.5(C₆H₆N₂O) | | C₁₉H₁₇F₆N₇O•C₇H₇NO₂ | |
| Fw | 665.50 | | 594.49 | | 534.44 | | 610.52 | |
| T [K] | 296(2)K | | 296(2)K | | 296(2)K | | 296(2)K | |
| λ [Å] | 1.54056 | | 1.54056 | | 1.54056 | | 1.54056 | |
| Crystal system | Monoclinic | | Monoclinic | | Monoclinic | | Monoclinic | |
| Proposed | P2₁/c | | C2 | | P2₁/n | | P2/c | |
| Space group | | | | | | | | |
| Unit cell | | | | | | | | |
| dimensions | | | | | | | | |
| a [Å] | 11.9799 | (2) | 27.7353 | (10) | 7.8589 | (3) | 15.9761 | (4) |
| b [Å] | 32.0689 | (5) | 5.3137 | (2) | 26.0399 | (7) | 14.7048 | (3) |
| c [Å] | 7.78839 | (8) | 19.1509 | (6) | 20.8153 | (6) | 11.8761 | (3) |
| α [°] | — | | — | | — | | — | |
| β [°] | 93.609 | (2) | 111.493 | (2) | 94.981 | (4) | 99.3510 | (15) |
| γ [°] | — | | — | | — | | — | |
| V [Å³] | 2986.23 | (8) | 2626.2 | (2) | 4243.7 | (2) | 2752.92 | (10) |
| Z (Z′) | 4 | (1) | 4 | (1) | 8 | (2) | 4 | (1) |

| | | | | |
|---|---|---|---|---|
| D_{c} [g/cm³] | 1.480 | 1.5030 | 1.670 | 1.473 |
| Cap. size [mm²] | 0.3 × 8 | 0.3 × 8 | 0.3 × 8 | 0.3 × 8 |
| 2θ Step size [°] | 0.16 | 0.16 | 0.16 | 0.16 |
| No of steps | 2441 | 2441 | 2441 | 2441 |
| Time per step [s] | 3 | 3 | 3 | 3 |
| 2θ range[°] | 3-41.5 | 3-41.5 | 3-41.5 | 3-41.5 |
| Rexp | 2.46 | 2.84 | 1.68 | 2.15 |
| R_{wp} | 2.93 | 4.68 | 2.96 | 3.63 |
| Rₚ | 2.25 | 3.54 | 2.04 | 2.64 |
| GOF | 1.19 | 1.65 | 1.76 | 1.69 |
| R_{Brag} | 0.10 | 0.33 | 0.16 | 0.28 |
| Impurities [%] | 7 (Citric | 0.5 (Benzoic | 8 (β form of | 0.5 (α Form |
| | acid) | acid) | Nicotinamide) | of 4- |
| | | | | Hydroxybenzamide) |

¹Empirical formula is a combination of the ¹H NMR data and calculated density of the crystal. The experimental conditions and NMR spectrum of the sample with nicotinamide suggests a stoichiometry of 1:1, the calculated cell parameters are questionable as based on the crystal density of the suggested crystal system/space group a ratio of 1:0.5 is more suitable.

i) Fumaric Acid Co-Crystal

Form Fum1 was formed by all three co-crystallization methods and remained stable for two days exposure to accelerated aging conditions. In many samples Fum1 was recovered with an excess of fumaric acid. However, when the ratio of Compound 1A and fumaric acid in the experiment was close to 1:0.5, pure Form Fum1 was obtained, suggesting that Fum1 could be a hemi co-crystal with a 1:0.5 stoichiometry. The HT-XRPD results of each experiment are described in Table 9. All samples were exposed to accelerated aging conditions (40° C./70% RH) for two days and re-analyzed by HT-XRPD (indicated in Table 8 as after AAC).

TABLE 9
Co-crystallization experiments with Compound 1A and fumaric acid. ‘lc’ stands for ‘low crystalline’,
‘tr’ stands for ‘trace’. Highlighted in green are the samples that resulted in a pure co-crystal

| | | Ratio | Solid phase | | Mother liquor | |
|---|---|---|---|---|---|---|
| Method | Solvent | API:CF | | After AAC | | After AAC |
| Melting | — | 1:1 | Fum0 + Fum1 | Fum0 + Fum1 | — | — |
| Melting | — | 1:0.5 | Fum1 | Not determined | — | — |
| Freeze drying | Tetrahydrofuran | 1:1 | lc | Fum1 | — | — |
| Sonication | Acetonitrile | 1:1 | Fum0 + Fum1 | Fum0 + Fum1 | — | — |
| Sonication | Ethyl acetate | 1:1 | Fum0 + Fum1 | Fum0 + Fum1 | — | — |
| Sonication | Chloroform | 1:1 | Fum0 + Fum1 | Fum0 + Fum1 | — | — |
| Solution | Ethyl acetate | 1:0.7 | Fum1 | Fum1 + tr Fum0 | No yield | — |
| Solution | Acetone/Water | 1:1.5 | Fum0 + Fum1 | Fum0 + Fum1 | No yield | — |
| | 90/10 | | | | | |
| Solution | THF/Water | 1:4.0 | Fum0 + Fum1 | Fum0 + Fum1 | Fum1 | Fum0 + Fum1 |
| | 85/15 | | | | | |
| Solution | 1,4-Dioxane | 1:2.3 | Fum0 + Fum1 | Fum0 + Fum1 | Fum0 + Fum1 | Fum0 + Fum1 |
| Solution | Ethanol/Water | 1:3.2 | Fum0 + Fum1 | Fum0 + Fum1 | Fum0 | Fum0 |
| | 83/17 | | | | | |
| Solution | Acetonitrile | 1:2.1 | Fum1 | Fum1 | Fum0 + Fum1 | Fum0 + Fum1 |

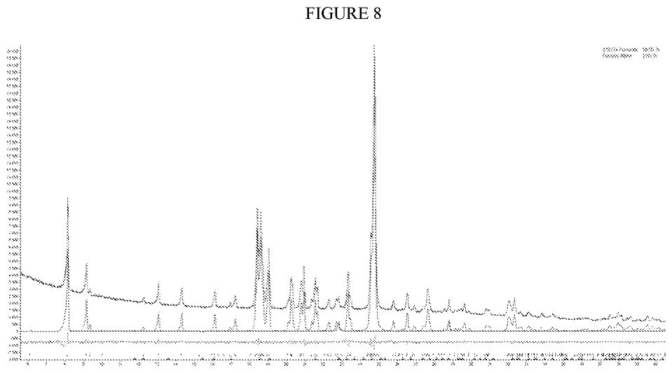

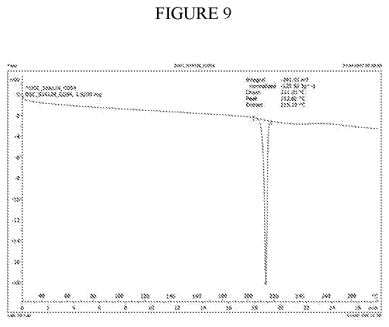

The sample obtained from the saturated solution method in ethyl acetate was used for the characterization of Fum1 (Exp TD C054). FIG. 7 provides an overlay of HT-XRPD patterns for Compound 1A, fumaric acid and Form Fum1 as obtained from the solvent mediated method in ethyl acetate (Exp ID CO54). FIG. 8 provides a graphical representation of the Whole Powder Pattern Decomposition of Form Fum1. FIG. 8 shows the recorded data, the calculated data and the difference between them. Also provided are the calculated powder pattern for co-crystal Fum1 and the calculated pattern for a form of fumaric acid. Based on the calculations, the sample contained 98% of co-crystal and 2% of fumaric acid. As indicated in FIG. 9, the thermal analysis shows that Form Fum1 has a melting temperature of 212.6° C. and is a non-solvated anhydrous form. The HPLC analysis confirmed the compound's integrity. The proton NMR spectrum provided in FIG. 9 confirmed that the molar ratio of Compound 1A:co-former in the co-crystal was 1:0.5, and a small excess of fumaric acid was present. Based on the Pawley refinement of the HR-XRPD data, the co-crystal probably crystallized in a monoclinic crystal with P21/c space group.

ii) Succinic Acid Co-Crystal

Form Suc1 was obtained with succinic acid from co-crystallization from saturated solution. Form Suc1 was found both as precipitated solids or recovered from the liquid phase. No unreacted Compound 1 A was found from the solution method. The sample obtained from the saturated solution method performed in acetonitrile was used for the characterization of Suc1 (Exp ID CO105). Table 10 provides the results of the co-crystallization experiments with Compound 1A and succinic acid. All samples were exposed to accelerated aging conditions (40° C./70% RH) for two days and re-analyzed by HT-XRPD (after AAC). The last row in Table 10 depicts the samples that resulted in a pure co-crystal.

TABLE 10
Results of the co-crystallization experiments with Compound 1A and succinic acid. ‘tr’ stands for ‘trace’.

| | | Ratio | Solid phase | | Mother liquor | |
|---|---|---|---|---|---|---|
| Method | Solvent | API:CF | | After AAC | | After AAC |
| Melting | — | 1:1 | — | — | — | — |
| Freeze drying | Tetrahydrofuran | 1:1 | Compound 1A, | Compound 1A, | — | — |
| | | | Form 1 | Form 1 | | |
| Sonication | Acetonitrile | 1:1 | Compound 1A, | Compound 1A, | — | — |
| | | | Form A + tr Suc1 | Form 1 + tr Suc1 | | |
| Sonication | Ethyl acetate | 1:1 | Compound 1A, | Compound 1A, | — | — |
| | | | Form 1 | Form 1 | | |
| Sonication | Chloroform | 1:1 | Compound 1A, | Compound 1A, | — | — |
| | | | Form 1 | Form 1 + Suc1 | | |
| Solution | Ethyl acetate | 1:0.8 | Suc1 + tr Suc0 | Suc1 + tr Suc0 | Suc0 + | Suc0 + |
| | | | | | Suc1 + Suc2 | Suc1 + Suc2 |
| Solution | Acetone/Water | 1:3.3 | Suc0 + Suc1 | Suc0 + Suc1 | Suc0 + Suc1 | Suc0 + Suc1 |
| | 90/10 | | | | | |
| Solution | THF/Water | 1:5.7 | Suc0 | Suc0 | Suc0 + Suc2 | Suc0 + Suc2 |
| | 85/15 | | | | | |
| Solution | 1,4-Dioxane | 1:5.0 | Suc0 | Suc0 | Suc0 + Suc1 | Suc0 + Suc1 |
| Solution | Ethanol/Water | 1:6.2 | Suc0 + Suc1 | Suc0 + Suc1 | Suc0 + Suc1 | Suc0 + Suc1 |
| | 83/17 | | | | | |
| Solution | Acetonitrile | 1:2.1 | Suc1 | Suc1 | Suc1 + Suc0 | Suc0 + |
| | | | | | | Suc1 + Suc2 |

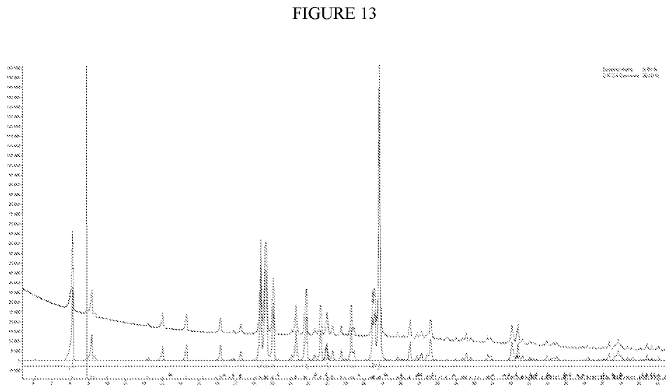

The thermal analysis confirmed that Suc1 is a non-solvated anhydrous form. FIG. 12 provides an overlay of HT-XRPD patterns for Compound 1A, succinic acid, Form Suc1 as obtained from the saturated solution method in acetonitrile (Exp ID CO105) and Suc2+Suc0 as obtained after evaporation of the mother liquor of the saturated solution method in tetrahydrofuran/water (85/15) (Exp ID CO75). FIG. 13 provides a graphical representation of the Whole Powder Pattern Decomposition (Pawley, G. S. (1981), J. Appl. Cryst., 14, 357-361) of Suc1 (Exp ID CO105). FIG. 13 provides the recorded data, calculated data and the difference between them. The calculated powder pattern for co-crystal Suc1 and the calculated pattern for a form of succinic acid are also provided. Based on the calculations, the sample contained 96% of co-crystal and 3.5% of succinic acid. The black vertical line shows a peak that was not indexed and most likely represents the presence of Compound 1A (˜0.5%).

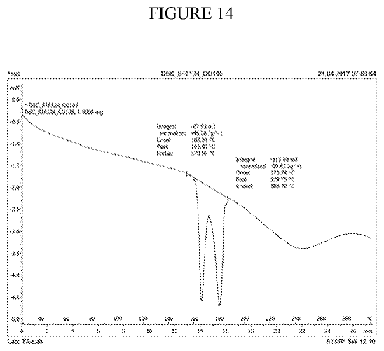

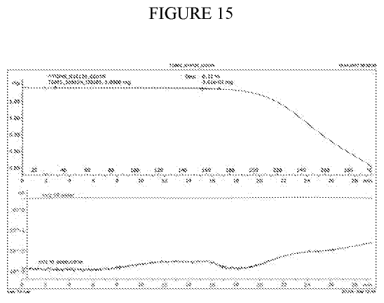

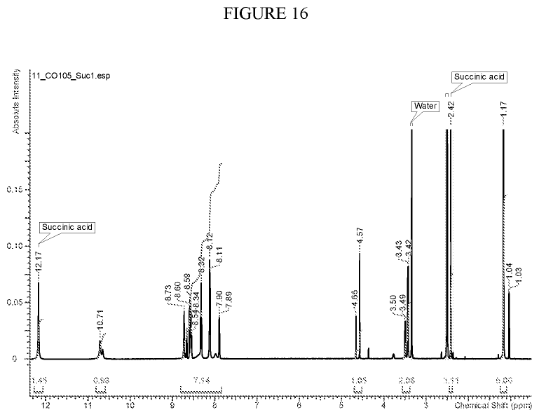

FIG. 14 provides the DSC spectrum obtained from the DSC analysis of Fum1 (Exp ID CO54) with a heating rate of 10° C./min and a pierced pan. Two endothermic events were recorded, at 156.8° C. and 179.8° C. Although the HT-XRPD pattern was distinct from the physical mixture of the API and succinic acid the thermal behavior was similar to the melting of a physical mixture of Compound 1A and co-former. The nature of the endothermic events was therefore not clear. FIG. 15 provides a TGMS spectrum with a heating rate of 10° C./min, of Suc1 (Exp ID CO105). The TGMS signal shows a mass loss of 0.33% prior to melt and decomposition. The HPLC analysis confirmed the compound's integrity. The 1H-NMR spectrum provided in FIG. 16 suggested that the stoichiometry of Compound 1A and succinic acid was 1:0.5. The HR XRPD data suggested that the co-crystal crystallized in a monoclinic crystal system with space group of P21/c.

Form Suc2 was only observed in mixtures with Suc1 or with succinic acid and hence this form was not characterized.

iii) Nicotinamide

Each applied method described above in Table 7 using nicotinamide resulted in the formation of co-crystals, although often in a mixture either with Compound 1A or with nicotinamide. The abundant form was Form Nic1. From the saturated solution method Forms Nic1 and Nic3 were obtained as pure forms and Form Nic2 was obtained in a mixture with nicotinamide. In most experiments co-crystallization took place, although in many samples an excess of nicotinamide was observed by XRPD analysis. All samples were exposed to accelerated aging conditions (40° C./70% RH) for two days and re-analyzed by HT-XRPD (after AAC). The highly crystalline forms with nicotinamide were stable for at least two days exposure to accelerated aging conditions. The XRPD results of each experiment are described in Table 11.

TABLE 11
Results of the co-crystallization experiments with Compound 1A and nicotinamide. When
samples were low crystalline this is denoted with ‘lc’, ‘tr’ stands for trace. .

| | | Ratio | Solid phase | | Mother liquor | |
|---|---|---|---|---|---|---|
| Method | Solvent | API:CF | | After AAC | | After AAC |
| Melting | — | 1:1 | Nic1 + tr | — | — | — |
| | | | Compound 1A, | | | |
| | | | Form 17 | | | |
| Freeze drying | Tetrahydrofuran | 1:1 | Nic3 lc | Nic0 + Nic1 | — | — |
| Sonication | Acetonitrile | 1:1 | Nic0 + Nic1 | Nic0 + Nic1 | — | — |
| Sonication | Ethyl acetate | 1:1 | FB2b | Compound 1A, | — | — |
| | | | | Form A | | |
| Sonication | Chloroform | 1:1 | Am | Compound 1A, | — | — |
| | | | | Form 1 | | |
| Solution | Ethyl acetate | 1:1.0 | Nic1 | Nic1 | No yield | — |
| Solution | Acetone/Water | 1:4.2 | Nic0 + Nic1 | Nic0 + Nic1 | No yield | — |
| | 90/10 | | | | | |
| Solution | THF/Water | 1:5.3 | Nic0 + Nic1 | Nic0 + Nic1 | No yield | — |
| | 85/15 | | | | | |
| Solution | 1,4-Dioxane | 1:0.8 | Nic3 | Nic3 | Compound 1A, | Compound 1A, |
| | | | | | Form G + Nic3 | Form G + |
| | | | | | | Compound 1A, |
| | | | | | | Form 1 + Nic3 |
| Solution | Ethanol/Water | 1:8.9 | Nic0 + Nic2 | Nic0 + Nic2 | Nic0 | Nic0 |
| | 83/17 | | | | | |
| Solution | Acetonitrile | 1:4.8 | Nic1 + tr Nic0 | Nic1 + tr Nic0 | Nic0 | Nic0 |

The sample obtained from the saturated solution method performed in ethyl acetate was used for the characterization of Form Nic1 (Exp ID CO57).

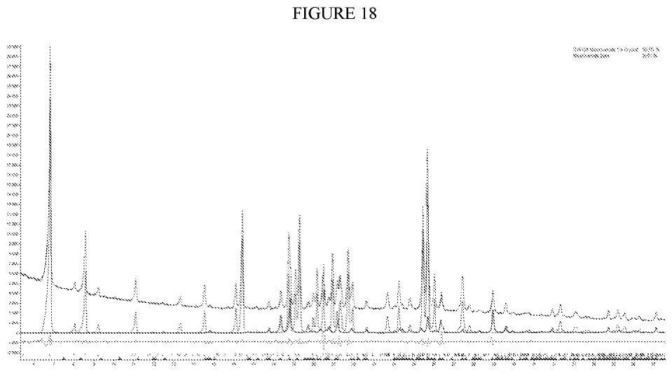

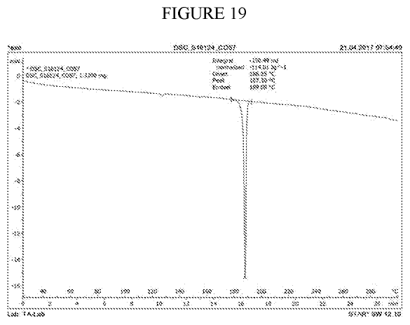

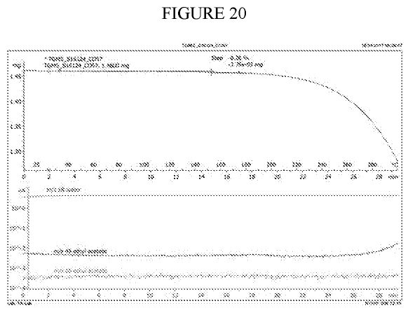

FIG. 17 provides an overlay of HT-XRPD patterns for Compound 1A, Nicotinamide, and Form Nic1 as obtained from the saturated solution method in ethyl acetate (Exp ID CO57), Forms Nic2+Nic0 as obtained from the saturated solution method in ethanol/water (73/17) (Exp ID CO97) and Form Nic3 as obtained from the saturated solution method in 1,4-dioxane (Exp ID CO87). FIG. 18 provides a graphical representation of the Whole Powder Pattern Decomposition (Pawley, G. S. (1981), J. Appl. Cryst., 14, 357-361) of Form Nic1 (Exp ID CO57). In FIG. 18, the recorded data, the calculated data and the difference between them is provided. Also indicated are the calculated powder pattern for co-crystal Nic1 and the calculated pattern for 3 form of nicotinamide. Based on the calculations, the sample contained 92% of co-crystal and 8% of nicotinamide. FIG. 19 provides a DSC spectrum of Form Nic1 (Exp ID CO57) with a heating rate of 10° C./min and a pierced pan. The thermal analysis suggested that Nic1 is a non-solvated anhydrous form with a melting point of 187.3° C. A TGMS spectrum of Nic1 (Exp ID CO57), with a heating rate of 10° C./min), is provided in FIG. 20. The TGMS signal shows a mass loss of 0.26% prior to melting. HPLC analysis confirmed the compound's integrity. The experimental conditions and 1H-NMR analysis of Form Nic1 pointed towards a ratio of Compound 1 Å:nicotinamide of 1:1. However, the density belonging to the cell parameters of the suggested crystal system calculated based on the Pawley refinement of the HR-XRPD data did not fit a 1:1 molar ratio of Compound 1A:nicotinamide, but rather 1:0.5.

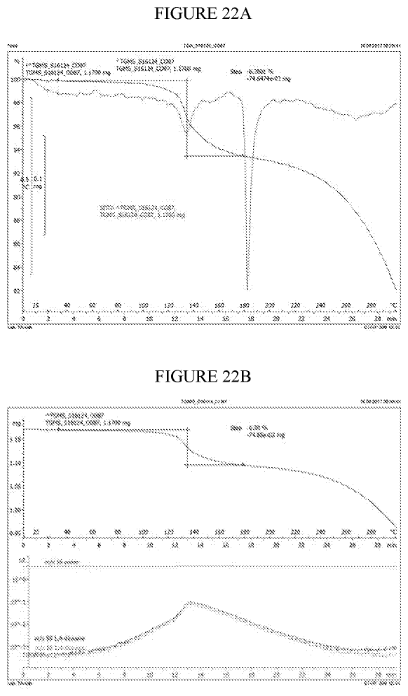

FIG. 22A provides a TGA/SDTA thermograms and FIG. 22B proxies a TGA/MS spectrum of the TGMS analysis (with a heating rate of 10° C./min) of Form Nic3 (Exp ID CO87). The SDTA shows two endothermic events. The first event around 136° C. coincides with a mass loss of 6.4% of 1,4-dioxane. The second endothermic event at 182° C. corresponds most likely to the melting of Form Nic1. The TGMS result suggested that Nic3 contained solvent. The thermal behavior after loss of the solvent was identical to the thermal behavior of Form Nic1, indicating that Form Nic3 converted to Form Nic1 after desolvation.

Form Nic2 was only obtained in a mixture with excess of nicotinamide and was not further characterized.

iv) Benzoic Acid

Form Ben1 was formed by all three methods described above in Table 7, and remained stable for two days during exposure to accelerated aging conditions. The melting and sonication experiments were performed in a molar ratio of Compound 1A and benzoic acid of 1:1 and resulted in pure Form Ben1. With the saturated solution method, a large excess of benzoic acid had to be used to force precipitation and all samples resulted in Form Ben1 with Form Ben0. The XRPD results of each experiment are described in Table 12. In all experiments co-crystallization took place, although in the samples obtained from the saturated solution method an excess of benzoic acid was observed by XRPD analysis. All samples were exposed to accelerated aging conditions (40° C./70% RH) for two days and re-analyzed by HT-XRPD (after AAC).

TABLE 12
Results of the co-crystallization experiments with Compound 1A and benzoic acid.

| | | Ratio | Solid phase | | Mother liquor | |
|---|---|---|---|---|---|---|
| Method | Solvent | API:CF | | After AAC | | After AAC |
| Melting | — | 1:1 | Ben1 | Ben1 | — | — |
| Freeze drying | Tetrahydrofuran | 1:1 | Ben1 | Ben1 | — | — |
| Sonication | Acetonitrile | 1:1 | Ben1 | Ben1 | — | — |
| Sonication | Ethyl acetate | 1:1 | Ben1 | Ben0 + Ben1 | — | — |
| Sonication | Chloroform | 1:1 | Ben1 | Ben1 | — | — |
| Solution | Ethyl acetate | 1:7.1 | Ben0 + Ben1 | Ben0 + Ben1 | Ben0 + | Ben0 + Ben1 |
| | | | | | trace Ben1 | |
| Solution | Acetone/Water | 1:6.6 | Ben0 + Ben1 | Ben0 + Ben1 | Ben0 + | Ben0 + Ben1 |
| | 90/10 | | | | trace Ben1 | |
| Solution | THF/Water | 1:14 | Ben0 + Ben1 | Ben0 + Ben1 | Ben0 + Ben1 | Ben0 + Ben1 |
| | 85/15 | | | | | |
| Solution | 1,4-Dioxane | 1:7.8 | — | — | Ben0 + Ben1 | Ben0 + Ben1 |
| Solution | Ethanol/Water | 1:9.9 | Ben0 + Ben1 | Ben0 + Ben1 | Ben0 + Ben1 | Ben0 + Ben1 |
| | 83/17 | | | | | |
| Solution | Acetonitrile | 1:9.6 | Ben0 + Ben1 | Ben0 + Ben1 | Ben0 + Ben1 | Ben0 + Ben1 |

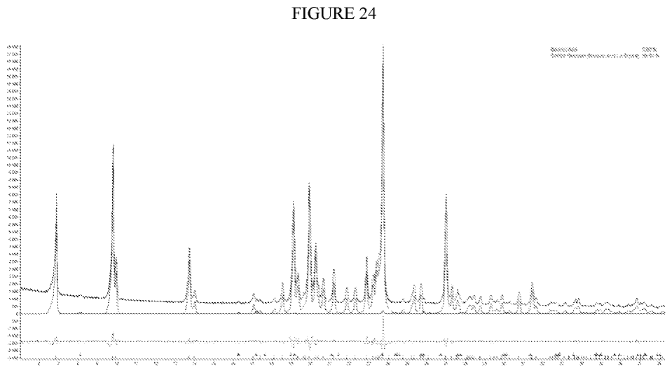

The sample obtained from sonication with acetonitrile was used for the characterization of Form Ben1 (Exp ID CO15). FIG. 23 provides an overlay of HT-XRPD patterns (from bottom to top): Compound 1A, benzoic acid and Form Ben1 as obtained from sonication in acetonitrile (Exp ID CO15). FIG. 24 provides a graphical representation of the Whole Powder Pattern Decomposition (Pawley, G. S. (1981), J. Appl. Cryst., 14, 357-361) of Form Ben1 (Exp ID CO15). In FIG. 24, the recorded data, the calculated data and the difference between them in provided. Also provided is the calculated powder pattern for co-crystal Ben1 and the calculated pattern for benzoic acid. Based on the calculations the sample contained 99.5% of co-crystal and 0.5% of benzoic acid.

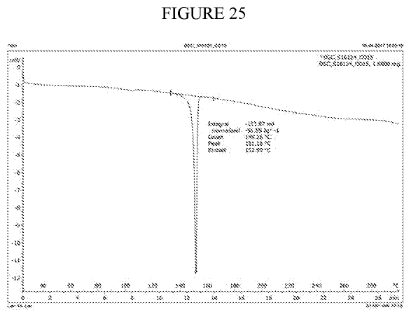

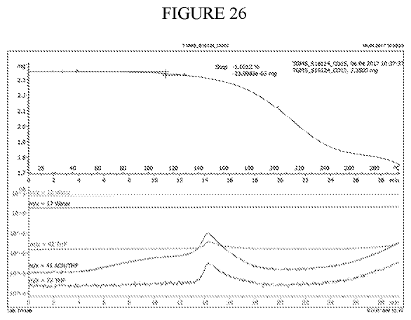

FIG. 25 provides a DSC analysis of Form Ben1 (Exp ID CO15). One single endothermic melting event was recorded at 151.2° C. confirming that Form Ben1 has a melting temperature of 151.2° C. and is a non-solvated anhydrous form. The TGMS analysis of Ben1 (Exp ID CO15), provided in FIG. 26, shows a mass loss of 1.0% prior to melting/decomposition. HPLC and ¹H-NMR analysis confirmed the compound's integrity. As seen from the ¹H-NMR spectrum, the stoichiometry of the co-crystal is most likely 1:1. The cell parameters of the crystal system were calculated based on Pawley refinement of the HR-XRPD data. The suggested crystal system is a monoclinic with C2 space group.

v) Uracil

With uracil one hit was found by the saturated solution method performed in acetonitrile. Form Ura1 remained stable for two days during exposure to accelerated aging conditions. All other experiments resulted in physical mixtures or separation of Compound A and uracil, most likely due to the poor solubility of uracil in most solvents. The XRPD results of each experiment are described in Table 13. All samples were exposed to accelerated aging conditions (40° C./70% RH) for two days and re-analyzed by HT-XRPD (after AAC). The samples in last rows are those resulted in a pure co-crystal.

TABLE 13
Results of the co-crystallization experiments with Compound 1A free base and uracil.

| | | Ratio | Solid phase | | | Mother liquor |
|---|---|---|---|---|---|---|
| Method | Solvent | API:CF | | After AAC | | After AAC |
| Melting | — | 1:1 | — | — | — | — |
| Freeze drying | Tetrahydrofuran | 1:1 | Ura0 | Compound 1A, | — | — |
| | | | | Form 1 + Ura0 | | |
| Sonication | Acetonitrile | 1:1 | Compound 1A, | Compound 1A, | — | — |
| | | | Form A + Ura0 | Form A + Ura0 | | |
| Sonication | Ethyl acetate | 1:1 | FB2b + Ura0 | Compound 1A, | — | — |
| | | | | Form A + Ura0 | | |
| Sonication | Chloroform | 1:1 | Ura0 | Compound 1A, | — | — |
| | | | | Form 1 + Ura0 | | |
| Solution | Ethyl acetate | 1:0.3 | — | — | Am | Compound 1A, |
| | | | | | | Form 1 |
| Solution | Acetone/Water | 1:0.4 | Compound 1A, | Compound 1A, | Compound 1A, | Compound 1A, |
| | 90/10 | | Form 1 + Ura0 | Form 1 + Ura0 | Form 1 | Form 1 |
| Solution | THF/Water | 1:0.4 | Ura0 | Ura0 | Compound 1A, | Compound 1A, |
| | 85/15 | | | | Form 1 | Form 1 |
| Solution | 1,4-Dioxane | 1:0.9 | Compound 1A, | Compound 1A, | Compound 1A, | Compound 1A, |
| | | | Form G + Ura0 | Form G + Ura0 | Form G | Form G + |
| | | | | | | Compound 1A, |
| | | | | | | Form 1 |
| Solution | Ethanol/Water | 1:0.4 | Compound 1A, | Compound 1A, | Compound 1A, | Compound 1A, |
| | 83/17 | | Form 1 + Ura0 | Form 1 + Ura0 | Form 1 | Form 1 |
| Solution | Acetonitrile | 1:0.6 | Ura1 | Ura1 | Compound 1A, | Compound 1A, |
| | | | | | Form 17 + | Form 17 + |
| | | | | | Compound 1A, | Compound 1A, |
| | | | | | Form A | Form A |

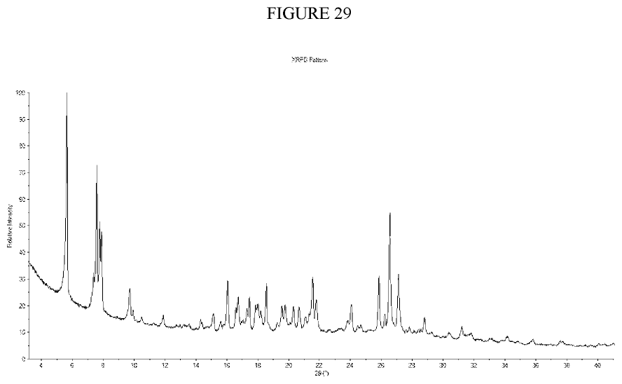

The sample obtained from the saturated solution method in acetonitrile was used for the characterization of Form Ura1 (Exp ID CO109). FIG. 28 provides an overlay of HT-XRPD patterns (from bottom to top): Compound 1A, uracil and Form Ura1 as obtained from the solution method in acetonitrile (Exp ID CO109). FIG. 29 provides a high resolution XRPD of Form Ura1 (Exp ID CO109).

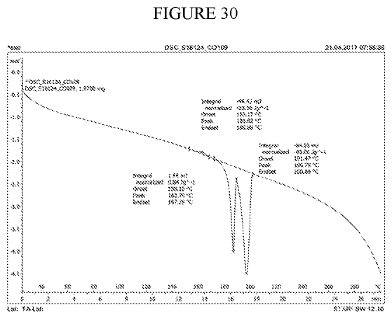

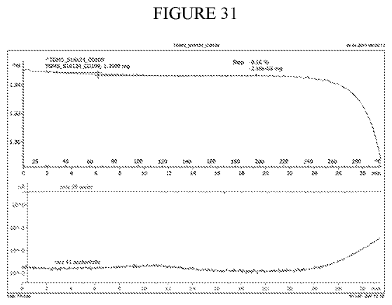

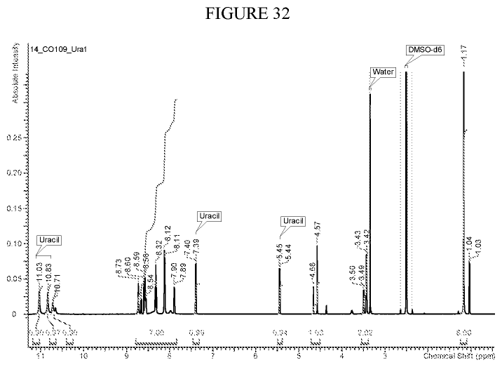

As depicted in FIG. 30, the DSC analysis showed a double endothermic event with Tpeaks of 187 and 197° C. FIG. 31 shows that no significant mass loss was observed by TGMS analysis. The nature of the double endothermic events was uncertain, but is possibly related to dissociation and melting. At above 230° C., degradation was observed. The HPLC analysis confirmed the compound's integrity. Based on the proton-NMR data in FIG. 32, the stoichiometric ratio was determined of Compound 1A:CF 1:1.

vi) Saccharin

With saccharin one potential co-crystal was observed. Form Sac1 was obtained by freeze drying and by the saturated solution method. During sonication Form Sac1 converted to a mixture of Form Sac1 and Compound 1A, but Form Sac1 was physically stable for two days exposure to accelerated aging conditions. The XRPD results of each experiment are described in Table 14. In the majority of the sonication and saturated solution method experiments co-crystallization took place. All samples were exposed to accelerated aging conditions (40° C./70% RH) for two days and re-analyzed by HT-XRPD (after AAC).

TABLE 14
Results of the co-crystallization experiments with Compound 1A and saccharin.

| | | Ratio | Solid phase | | Mother liquor | |
|---|---|---|---|---|---|---|
| Method | Solvent | API:CF | | After AAC | | After AAC |
| Melting | — | 1:1 | — | — | — | — |
| Freeze drying | Tetrahydrofuran | 1:1 | Sac1 | Sac1 | — | — |
| Sonication | Acetonitrile | 1:1 | Compound 1A, | Compound 1A, | — | — |
| | | | Form A + Sac1 | Form A + Sac1 | | |
| Sonication | Ethyl acetate | 1:1 | FB2b + Sac1 | Compound 1A, | — | — |
| | | | | Form A + Sac1 | | |
| Sonication | Chloroform | 1:1 | Compound 1A, | Compound 1A, | — | — |
| | | | Form 1 + Sac1 | Form 1 + Sac1 | | |
| Solution | Ethyl acetate | 1:1.4 | Sac1 | Sac1 | Sac1 | Sac1 + tr |
| | | | | | | Compound 1A, |
| | | | | | | Form 1 |
| Solution | Acetone/Water | 1:2.0 | Sac1 | Sac1 | Sac1 | Sac1 |
| | 90/10 | | | | | |
| Solution | THF/Water | 1:4.5 | Sac0 | Sac0 | Sac0 | Compound 1A, |
| | 85/15 | | | | | Form 1 + Sac0 |
| Solution | 1,4-Dioxane | 1:4.3 | — | — | Compound 1A, | Compound 1A, |
| | | | | | Form G + Sac0 | Form G + |
| | | | | | | Compound 1A, |
| | | | | | | Form 1 + Sac0 |
| Solution | Ethanol/Water | 1:1.4 | Sac1 | Sac1 | Sac1 | Sac1 |
| | 83/17 | | | | | |
| Solution | Acetonitrile | 1:4.7 | Sac1 + tr Sac0 | Sac1 + tr Sac0 | Sac1 + tr Sac0 | Sac0 + Sac1 |

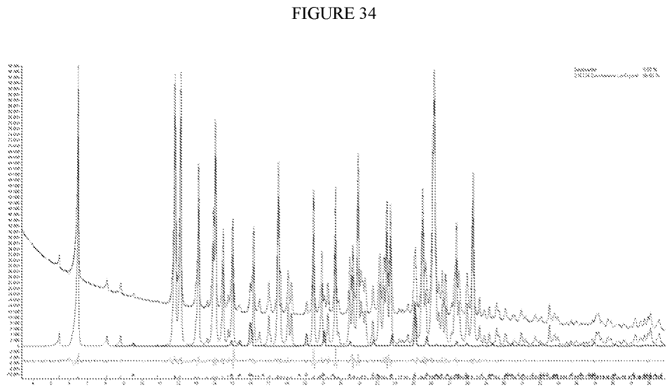

The sample obtained from the saturated solution method done in acetone/water (90/10) was used for the characterization of Form Sac1 (Exp ID CO70). FIG. 33 provides an overlay of HT-XRPD patterns (from bottom to top): Compound 1A, saccharin and Form Sac1 as obtained from the solution method in acetone/water (90/10) (Exp ID CO70). FIG. 34 provides a graphical representation of the Whole Powder Pattern Decomposition (Pawley, G. S. (1981), J. Appl. Cryst., 14, 357-361) of Form Sac1 (Exp ID CO70). In FIG. 34, the recorded data, the calculated data and the difference between them is provided. Also depicted are the calculated powder pattern for co-crystal Form Sac1 and the calculated pattern for saccharin. Based on the calculations the sample contained 96.5% of co-crystal and 3.5% of saccharin.

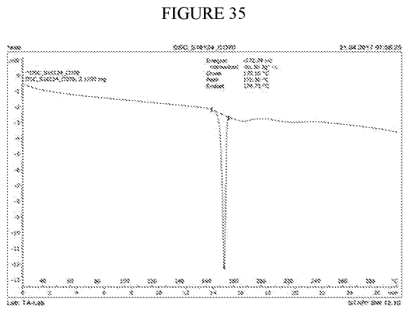

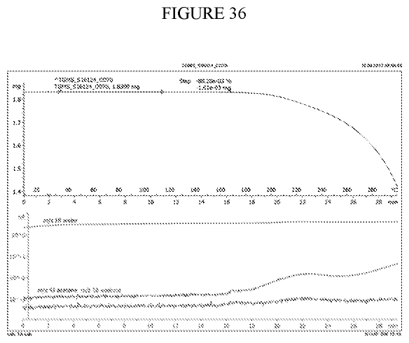

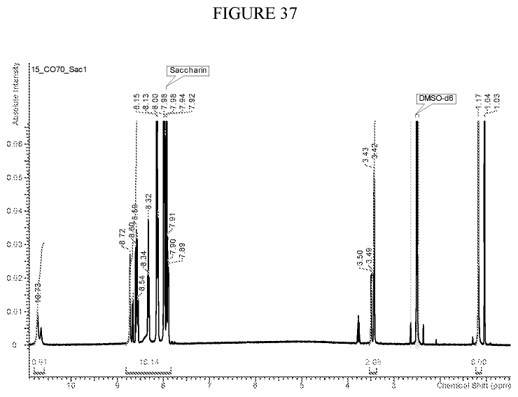

As seen in FIG. 35, the DSC spectrum showed that Form Sac1 has a melting temperature of 172.3° C. The TGMS spectrum in FIG. 36 showed no significant mass loss, proving that it is a non-solvated anhydrous form. The HPLC analysis confirmed the compound's integrity. The proton-NMR spectrum provided in FIG. 37 suggested a stoichiometric ratio of API:saccharin of 1:2. Indexing of the HR-XRPD data led to the suggested triclinic crystal system with space group P-1. The cell parameters were calculated according to the Pawley refinement.

vii) Citric Acid

With citric acid several co-crystal forms were obtained. The physically stable forms Cit3 and Cit4 were obtained from the saturated solution method. By sonication the physically unstable forms Cit1 and Cit2 were formed, conversions of Cit1 and 2 to Cit4 were observed upon exposure to AAC. The XRPD results of each experiment are described in Table 15. In the majority of experiments, co-crystallization took place. All samples were exposed to accelerated aging conditions (40° C./70% RH) for two days and re-analyzed by HT-XRPD (after AAC).

TABLE 15
Results of the co-crystallization experiments with Compound 1A and saccharin

| | | Ratio | Solid phase | | Mother liquor | |
|---|---|---|---|---|---|---|
| Method | Solvent | API:CF | | After AAC | | After AAC |
| Melting | — | 1:1 | — | — | — | — |
| Freeze drying | Tetrahydrofuran | 1:1 | Sac1 | Sac1 | — | — |
| Sonication | Acetonitrile | 1:1 | Compound 1A, | Compound 1A, | — | — |
| | | | Form A + Sac1 | Form A + Sac1 | | |
| Sonication | Ethyl acetate | 1:1 | FB2b + Sac1 | Compound 1A, | — | — |
| | | | | Form A + Sac1 | | |
| Sonication | Chloroform | 1:1 | Compound 1A, | Compound 1A, | — | — |
| | | | Form 1 + Sac1 | Form 1 + Sac1 | | |
| Solution | Ethyl acetate | 1:1.4 | Sac1 | Sac1 | Sac1 | Sac1 + tr |
| | | | | | | Compound 1A, |
| | | | | | | Form 1 |
| Solution | Acetone/Water | 1:2.0 | Sac1 | Sac1 | Sac1 | Sac1 |
| | 90/10 | | | | | |
| Solution | THF/Water | 1:4.5 | Sac0 | Sac0 | Sac0 | Compound 1A, |
| | 85/15 | | | | | Form 1 + Sac0 |
| Solution | 1,4-Dioxane | 1:4.3 | — | — | Compound 1A, | Compound 1A, |
| | | | | | Form G + Sac0 | Form G + |
| | | | | | | Compound 1A, |
| | | | | | | Form 1 + Sac0 |
| Solution | Ethanol/Water | 1:1.4 | Sac1 | Sac1 | Sac1 | Sac1 |
| | 83/17 | | | | | |
| Solution | Acetonitrile | 1:4.7 | Sac1 + tr Sac0 | Sac1 + tr Sac0 | Sac1 + tr Sac0 | Sac0 + Sac1 |

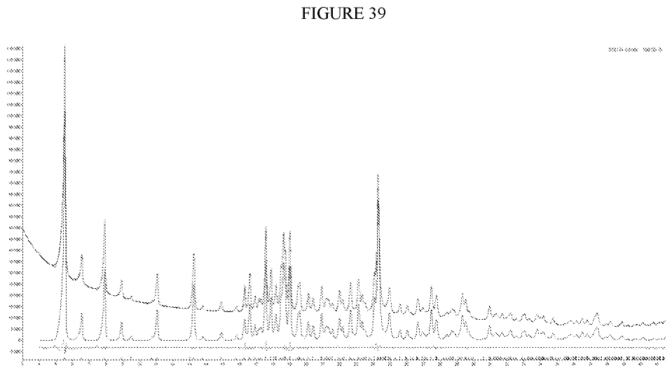

The sample obtained from the saturated solution method in ethyl acetate was used for the characterization of Cit3 (Exp ID CO61). FIG. 38 provides an overlay of HT-XRPD patterns (from bottom to top): Compound 1A, citric acid, Form Cit1 as obtained after sonication with acetonitrile (Exp ID CO18), Form Cit2 as obtained after sonication with chloroform (Exp ID CO49), Form Cit3 as obtained from the solution method in ethyl acetate (Exp ID CO61) and Form Cit4 as obtained from the solution method in acetonitrile (Exp TD CO111). FIG. 39 provides the graphical representation of the Whole Powder Pattern Decomposition (Pawley, G. 5. (1981), J. Appl. Cryst., 14, 357-361) of Form Cit3 (Exp TD C061). FIG. 39 provides the recorded data, the calculated data and the difference between them. FIG. 39 also shows the calculated powder pattern for co-crystal Cit3. Based on the calculations the sample consisted of pure co-crystal.

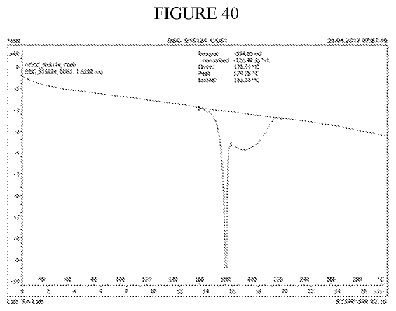

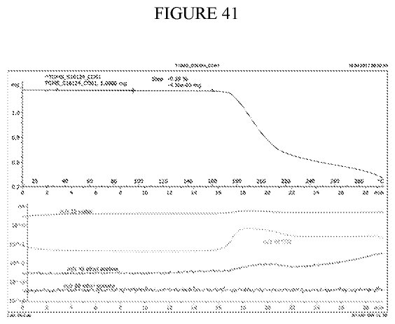

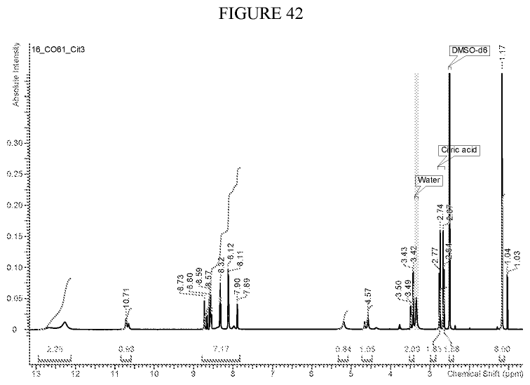

FIG. 40 provides a DSC spectrum of Form Cit3 (Exp ID CO61). An endothermic melting event was observed at 172.3° C., followed immediately by decomposition. A TGMS spectrum of Form Cit3 is provided in FIG. 41. A mass loss of 0.39% was observed prior to melting/decomposition. HPLC analysis confirmed the compound's integrity. The 1H-NMR spectrum provided in FIG. 42 reflected a ratio of Compound 1A:citric acid of 1:0.9.

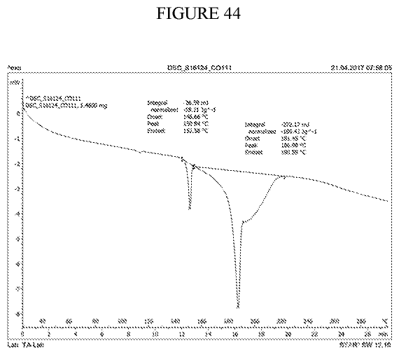

FIG. 44 provides a graphical representation of the Whole Powder Pattern Decomposition (Pawley, G. S. (1981), J. Appl. Cryst., 14, 357-361) of Cit4 (Exp ID CO11) in which the recorded data, the calculated data, and the difference between them is depicted. FIG. 44 also depicts the calculated powder pattern for co-crystal Cit4 and the calculated pattern for citric acid. Based on the calculations the sample contained 93% of co-crystal and 7% of citric acid. The data suggests a monoclinic crystal system with P21/c space group.

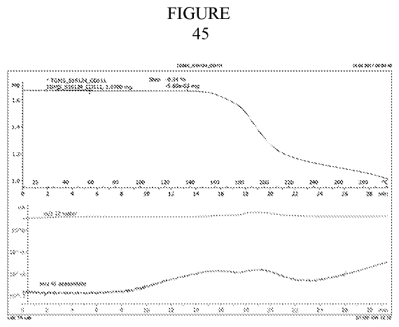

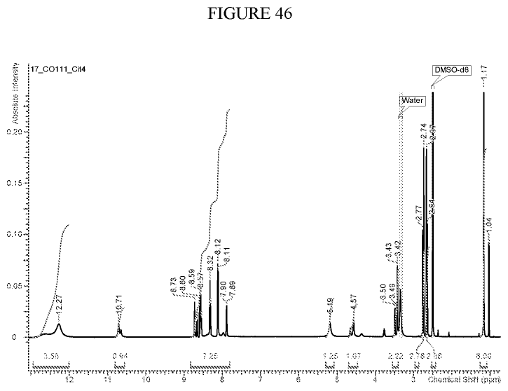

The sample obtained from the saturated solution method in acetonitrile was used for the characterization of Cit4 (Exp ID CO111). FIG. 44 provides a DSC spectrum of Form Cit4 (Exp ID CO111). A sharp endothermic event was observed at 150.8° C. followed by the melting and decomposition at 187.0° C., followed immediately by decomposition. FIG. 45 provides a TGMS spectrum of Form Cit4 (Exp ID CO111). A mass loss of 0.34% was observed prior to melting/decomposition indicating Form Cit4 as a non-solvated anhydrous form. The ¹H-NMR spectrum provided in FIG. 46 suggested a ratio of API:citric acid of 1:1.4. The compound's integrity was confirmed by HPLC analysis.

Forms Cit1 and Cit2 were unstable and as conversion to Form Cit4 was observed during exposure to AAC these forms were not further characterized.

viii) Lactamide

With lactamide different forms were identified depending on the co-crystallization method that was used. By melting/re-crystallization of a physical mixture of the API and lactamide Form Lac was obtained. With sonication Form Lac2 was formed and by the saturated solution method mixtures of Lac3 and lactamide were recovered. Form Lac and mixture Lac3+Lac0 were physically stable for 2 days at AAC. Lac2 was unstable and partially disproportionated to a mixture of Lac1+Lac3+Lac0. The XRPD results of each experiment are described in Table 16. All samples were exposed to accelerated aging conditions (40° C./70% RH) for two days and re-analyzed by HT-XRPD (after AAC).

TABLE 16
Results of the co-crystallization experiments with Compound 1A and lactamide.

| | | Ratio | Solid phase | | Mother liquor | |
|---|---|---|---|---|---|---|
| Method | Solvent | API:CF | | After AAC | | After AAC |
| Melting | — | 1:1 | Lac1 | Lac1 | — | — |
| Freeze drying | Tetrahydrofuran | 1:1 | Am | Lac4 | — | — |
| Sonication | Acetonitrile | 1:1 | Lac2 | Lac0 + | — | — |
| | | | | Lac2 + Lac3 | | |
| Sonication | Ethyl acetate | 1:1 | Lac2 | Lac0 + | — | — |
| | | | | Lac2 + Lac3 | | |
| Sonication | Chloroform | 1:1 | Am | Lac0 + Lac3 | — | — |
| Solution | Ethyl acetate | 1:1.8 | Lac0 + Lac3 | Lac0 + Lac3 | Lac0 + Lac3 | Lac0 + Lac3 |
| Solution | Acetone/Water | 1:4.8 | Lac0 + Lac3 | Lac0 + Lac3 | Lac0 + Lac3 | Lac0 + Lac3 |
| | 90/10 | | | | | |
| Solution | THF/Water | 1:10 | Lac0 + Lac3 | Lac0 + Lac3 | Lac0 + tr Lac3 | Lac0 + tr Lac3 |
| | 85/15 | | | | | |
| Solution | 1,4-Dioxane | 1:4.7 | Lac0 + Lac3 | Lac0 + Lac3 | Lac0 + Lac3 | Lac0 + Lac3 |
| Solution | Ethanol/Water | 1:13 | Lac0 + Lac3 | Lac0 + Lac3 | Lac0 + tr Lac3 | Lac0 + tr Lac3 |
| | 83/17 | | | | | |
| Solution | Acetonitrile | 1:11 | Lac0 + Lac3 | Lac0 + Lac3 | Lac0 + tr Lac3 | Lac0 + tr Lac3 |

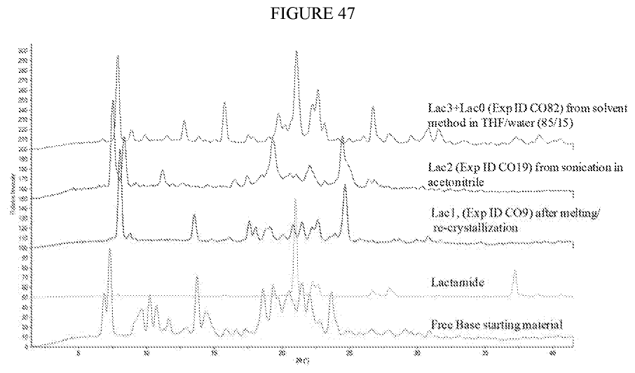

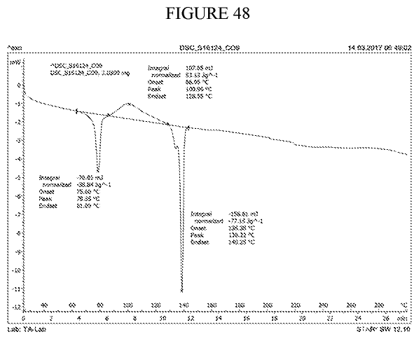

FIG. 47 provides an overlay of HT-XRPD patterns (from bottom to top): Compound 1A, lactamide, Form Lac1 as obtained from the co-melting experiment (Exp ID CO9), Form Lac2 as obtained from sonication with acetonitrile (Exp TD CO19) and Forms Lac3+Lac0 as obtained from the saturated solution method in tetrahydrofuran/water (85/15) (Exp ID CO82). The DSC result provided in FIG. 48 showed a melting temperature of the co-crystal at 138° C. A small shoulder was observed in the endothermic melting event. Form Lad1 is a solvent free form as the co-crystal was obtained by co-melting without addition of solvent.

No other analyses were performed on Lac2 and Lac3 as the forms were not stable or only obtained in a mixture with excess of lactamide.

ix) Hydroxybenzamide

With 4-hydroxybenzamide several polymorphs of the co-crystal were obtained. The XRPD results of each experiment are described in Table 17. Form Hbe1 was found from all three methods and appeared to be physically stable during exposure to accelerated aging conditions. Form Hbe2 was obtained from freeze drying and partly converted to Form HiBe1 during sonication. Upon exposure to AAC this form completely converted to Form Hbe1.

TABLE 17
Results of the co-crystallization experiments with Compound 1A and 4 hydroxybenzamide

| | | Ratio | Solid phase | | Mother liquor | |
|---|---|---|---|---|---|---|
| Method | Solvent | API:CF | | After AAC | | After AAC |
| Melting | — | 1:1 | Hbe1 | Hbe1 | — | — |
| Freeze drying | Tetrahydrofuran | 1:1 | Hbe2 | Hbe1 | — | — |
| Sonication | Acetonitrile | 1:1 | Hbe1 + Hbe2 | Hbe1 | — | — |
| Sonication | Ethyl acetate | 1:1 | Hbe1 + Hbe2 | Hbe1 | — | — |
| Sonication | Chloroform | 1:1 | Hbe1 + Hbe2 | Hbe1 | — | — |
| Solution | Ethyl acetate | 1:1.2 | Hbe1 | Hbe1 | Hbe0 + Hbe1 | Hbe0 + Hbe1 |
| Solution | Acetone/Water | 1:4.7 | Hbe0 + Hbe3 | Hbe3 | No yield | — |
| | 90/10 | | | | | |
| Solution | THF/Water | 1:4.1 | FB7 + Hbe1 | FB7 + Hbe1 | No yield | — |
| | 85/15 | | | | | |
| Solution | 1,4-Dioxane | 1:1.0 | Hbe0-2 | Hbe0 + tr Hbe1 | Compound 1A, | Compound 1A, |
| | | | | | Form G | Form G + |
| | | | | | | Compound 1A, |
| | | | | | | Form 1 |
| Solution | Ethanol/Water | 1:4.3 | Hbe0 + | Hbe0 + | Hbe0-1 | Hbe0-1 |
| | 83/17 | | Hbe1 + Hbe2 | Hbe1 + Hbe2 | | |
| Solution | Acetonitrile | 1:3.0 | Hbe1 | Hbe1 | Hbe0 + | Hbe0 + Hbe1 |
| | | | | | Hbe1 + Hbe3 | |

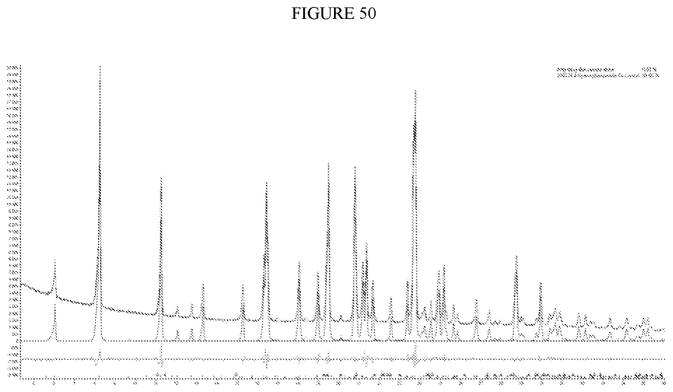

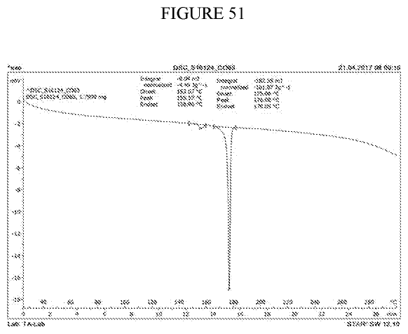

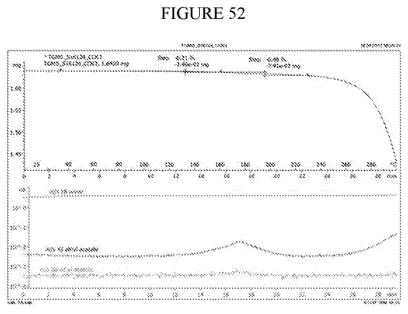

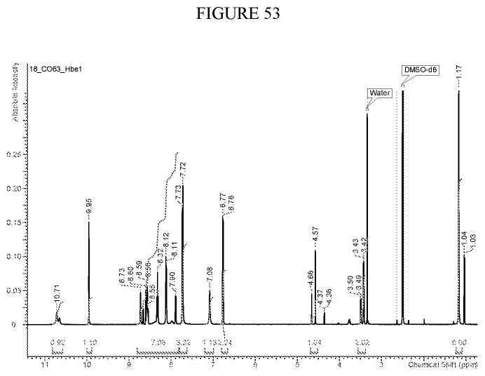

Form Hbe1 obtained from the solution method in ethyl acetate (Exp ID CO63) was used for characterization. FIG. 49 provides an overlay of HT-XRPD patterns (from bottom to top): Compound 1A, 4-hydroxybenzamide, Form libel as obtained from the saturated solution method in ethyl acetate (Exp ID CO63), Form Hbe2 as obtained from freeze drying from THE (Exp ID CO53) and Forms Hbe3+Hbe0 as obtained from the saturated solution method in acetone/water (90/10) (Exp TD CO73). FIG. 50 provides a graphical representation of the Whole Powder Pattern Decomposition (Pawley, G. S. (1981), J. Appl. Cryst., 14, 357-361) of Hbe1 (Exp ID CO63) in which the recorded data, the calculated data and the difference between them is depicted. FIG. 50 also depicts the calculated powder pattern for co-crystal libel and the calculated pattern for α form of 4-hydroxybenzamide. Based on the calculations, the sample contained 99.500 of co-crystal and 0.5% of 4-hydroxybenzamide. FIG. 51 provides a DSC spectrum of Form libel (Exp ID CO63) showing an endothermic melting event at 176.1° C., a small endothermic event prior to melting was observed at 155.6° C. FIG. 52 provides a TGMS spectrum of Form Hbe1 (Exp ID CO63). The TGMS signal shows a mass loss of 0.21% prior to melting. The HPLC analysis confirmed the compound's integrity. The ¹H-NMR spectrum provided in FIG. 53 confirmed that the ratio of Compound 1A and 4-hydroxybenzamide was 1:1.

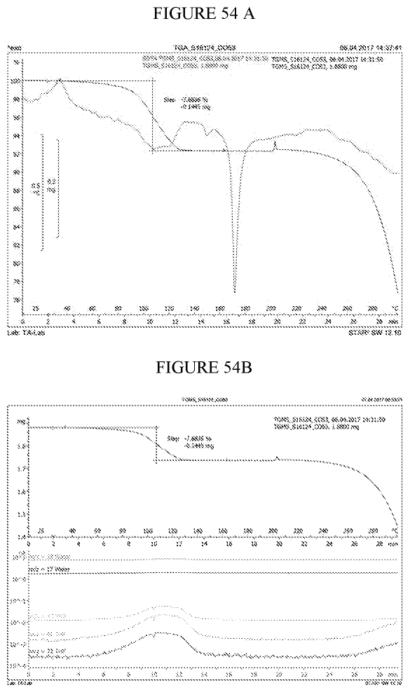

Form Hbe2, obtained from the freeze drying experiment (Exp ID CO20) was analyzed by TGMS and showed a mass loss of 7.7% as seen in FIG. 54. After the mass loss the thermal behavior was identical to the thermal behavior of Form Hbe1.

Form Hbe1 was a non-solvated and anhydrous form, whereas Hbe2 was a solvate that converted to Hbe1 upon desolvation.

### Example 4: Synthesis of Compound 1A Form A

Slow evaporation of Compound 1A, Form 1 was performed in 10 solvent systems by dissolving 5-10 mg of Compound 1A, Form 1 in 1.0-2.0 mL of acetone or acetonitrile in a 3-mL glass vial. The visually clear solutions were covered with caps and subjected to slow evaporation to induce precipitation at room temperature. The solids were isolated for XRPD analysis after the samples were evaporated to dryness.

The XRPD pattern of Form A is provided in FIG. 55. A DSC profile of Form A is provided in FIG. 56. The DSC profile is characterized by one endotherm at 168.5° C. (onset temperature). A TGA profile of Form A is shown in FIG. 56. The weight loss in thermal gravimetric analysis represents a loss of about 3.8% of the weight of the sample as the temperature is increased to about 160.0° C. FIG. 57 provides the ¹H NMR spectrum, which indicates that the molar ratio of acetone to Form A is 0.06 due to the existence of residual solvent.

### Example 5: Synthesis of Compound 1A Form G

About 10 mg of Compound 1A, Form 1 was dissolved in dioxane to obtain a clear solution to obtain a clear solution, followed by addition of n-heptane anti-solvent (ratio of dioxane/n-heptane 1:4). After stirring for about 24 hours, the precipitates were isolated for XRPD analysis and the clear solutions were subjected to slow evaporation to dryness. The solids were isolated for XRPD analysis after the samples were evaporated to dryness.

The XRPD pattern of Form G is provided in FIG. 58. A DSC profile of Form G is provided in FIG. 59. The DSC profile is characterized by two endotherms at 114.3° C. and 204.9° C. (onset temperature). A TGA profile of Form G is shown in FIG. 59. The weight loss in thermal gravimetric analysis represents a loss of about 12.4% of the weight of the sample as the temperature is increased to about 161.0° C. When heated to 160° C., Form G converted to an amorphous form, as shown in FIG. 59. FIG. 60 provides the ¹H NMR spectrum, which indicates that the molar ratio of dioxane to Form G is 0.5. The NMR data combined with the TGA data indicate that Form G may be dioxane solvate.

### Example 6: Synthesis of Compound 1A Form K

Compound 1A, Form 2 after dynamic vapor sorption (DVS) provided Form K. An XRPD pattern of Form K is provided in FIG. 61. A DSC profile of Form K is provided in FIG. 59. The DSC profile is characterized by two endotherms at 38.7° C. and 117.7° C. (onset temperature). A TGA profile of Form K is shown in FIG. 62. The weight loss in thermal gravimetric analysis represents a loss of about 1.9% of the weight of the sample as the temperature is increased to about 66° C. The TGA data indicate that Form K may be an anhydrate.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A co-crystal comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol and a coformer selected from a group consisting of fumaric acid, succinic acid, benzoic acid, citric acid, nicotinamide, lactamide, 4-hydroxybenzamide, uracil, and saccharin.

2. The co-crystal of claim 1, wherein the coformer is fumaric acid.

3. The co-crystal of claim 2, wherein the co-crystal is Form Fum1 having an X-ray powder diffraction pattern substantially similar to the pattern presented in FIG. 7.

4. The co-crystal of claim 1, wherein the coformer is succinic acid.

5. The co-crystal of claim 4, wherein the co-crystal is Form Suc1 having an X-ray powder diffraction pattern substantially similar to the pattern presented in FIG. 12.

6. The co-crystal of claim 1, wherein the coformer is benzoic acid.

7. The co-crystal of claim 6, wherein the co-crystal is Form Ben1 having an X-ray powder diffraction pattern substantially similar to the pattern presented in FIG. 23.

8. The co-crystal of claim 1, wherein the coformer is citric acid.

9. The co-crystal of claim 8, wherein the co-crystal is Form Cit3 having an X-ray powder diffraction pattern substantially similar to the pattern presented in FIG. 38.

10. The co-crystal of claim 8, wherein the co-crystal is Form Cit4 having an X-ray powder diffraction pattern substantially similar to the pattern presented in FIG. 38.

11. The co-crystal of claim 1, wherein the coformer is nicotinamide.

12. The co-crystal of claim 11, wherein the co-crystal is Form Nic1 having an X-ray powder diffraction pattern substantially similar to the pattern presented in FIG. 17.

13. The co-crystal of claim 1, wherein the coformer is lactamide.

14. The co-crystal of claim 13, wherein the co-crystal is Form Lac having an X-ray powder diffraction pattern substantially similar to the pattern presented in FIG. 47.

15. The co-crystal of claim 1, wherein the coformer is 4-hydroxybenzamide.

16. The co-crystal of claim 15, wherein the co-crystal is Form Hbe1 having an X-ray powder diffraction pattern substantially similar to the pattern presented in FIG. 49.

17. The co-crystal of claim 1, wherein the coformer is uracil.

18. The co-crystal of claim 17, wherein the co-crystal is Form Ura1 having an X-ray powder diffraction pattern substantially similar to the pattern presented in FIG. 28.

19. The co-crystal of claim 1, wherein the coformer is saccharin.

20. The co-crystal of claim 19, wherein the co-crystal is Form Sac1 having an X-ray powder diffraction pattern substantially similar to the pattern presented in FIG. 34.

21. The co-crystal of claim 1, wherein the molar ratio of 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol to the coformer is about 1:1.

22. The co-crystal of claim 1, which is substantially crystalline.

23. A pharmaceutical composition comprising the co-crystal of claim 1, and a pharmaceutically acceptable excipient.

24. A method of treating a disease selected from a hematological malignancy and a solid tumor, each characterized by the presence of a mutant allele of IDH2, comprising administering to a subject having the disease, a therapeutically effective amount of the co-crystal of claim 1.

25. The method of claim 24, wherein the disease is characterized by the presence of a mutant allele of IDH2 and the absence of a mutant allele of FLT3.

26. The method of claim 24, wherein the disease is characterized by the presence of a mutant allele of IDH2 and the absence of a mutant allele of NRAS.

27. The method of claim 24, wherein the disease is a hematological malignancy.

28. The method of claim 24, wherein the hematological malignancy is selected from the group consisting of acute myelogenous leukemia, myelodysplastic syndrome, chronic myelomonocytic leukemia myeloid sarcoma, multiple myeloma, lymphoma, angioimmunoblastic T-cell lymphoma, blastic plasmacytoid dendritic cell neoplasm and myeloproliferative neoplasm, each characterized by the presence of a mutant allele of IDH2.

29. The method of claim 24, wherein the hematological malignancy is acute myelogenous leukemia.

30. The method of claim 24, wherein the disease is myelodysplastic syndrome.

31. The method of claim 30, wherein the disease is characterized by the presence of a mutant allele of IDH2 and a mutant allele of at least one second gene, wherein the second gene is selected from the group consisting of ASXL1 and SRSF2.

32. The method of claim 30, wherein the disease is characterized by the presence of a mutant allele of IDH2 and the absence of a mutant allele of at least one other gene, wherein the other gene is selected from the group consisting of KRAS, TP53, SETBP1, U2AF1, TCF3, STAG2, NRAS, JAK2 and BRAF.

33. The method of claim 24, wherein the solid tumor is selected from the group consisting of from glioma, melanoma, chondrosarcoma, and cholangiocarcinoma, each characterized by the presence of a mutant allele of IDH2.

34. The method of claim 24, wherein the disease is relapsed or refractory.

35. The method of claim 24, further comprising administering a second active agent.

36. The method of claim 24, wherein the subject is a pediatric patient.

37-49. (canceled)
